(19) United States
(12) Patent Application Publication
(10) Pub. No.: US 2024/0368642 A1
(43) Pub. Date: Nov. 7, 2024

(54) COMPOSITIONS AND METHODS FOR PRODUCTION OF VALUE-ADDED CHEMICALS

(71) Applicant: Solugen, Inc., Houston, TX (US)

(72) Inventors: Toni M. Lee, Missouri City, TX (US); Brian F. Fisher, Houston, TX (US); Philipp Wiemann, Houston, TX (US); Gaurab Chakrabarti, Houston, TX (US); Peter Nguyen, Ssaugus, CA (US); Kevin Loftis, Spring, TX (US); Shuai Qian, Houston, TX (US); Konrad Miller, Sugar Land, TX (US); Hans-Joerg Woelk, Hoouston, TX (US); Sarah Downing, Houston, TX (US); David Weiner, Houston, TX (US); Danielle Fair, Houston, TX (US); Sean Hunt, Houston, TX (US)

(73) Assignee: Solugen, Inc., Houston, TX (US)

(21) Appl. No.: 18/580,308

(22) PCT Filed: Jul. 22, 2022
(86) PCT No.: PCT/US2022/074076

§ 371 Date: Jan. 18, 2024

Related U.S. Application Data
(60) Provisional application No. 63/224,553, filed on Jul. 22, 2021.

(51) Int. Cl. C12P 7/40; B01J 23/72; B01J 27/02; B01J 29/40; B01J 29/70; C12N 9/04; C12N 9/08; C12N 9/88; C12P 7/02; C12P 7/24

(52) U.S. Cl. CPC C12P 7/40; B01J 23/72; B01J 27/02; B01J 29/40; B01J 29/70; C12N 9/0006; C12N 9/0065; C12N 9/88; C12P 7/02; C12P 7/24; C12Y 101/03004; C12Y 101/03009; C12Y 101/0301; C12Y 111/02001; C12Y 402/0104

(57) ABSTRACT

A molecular manufacturing process includes contacting a platform molecule with (i) a biocatalyst and (ii) a chemical catalyst under conditions suitable to produce a value-added chemical.

### CROSS-REFERENCE TO RELATED APPLICATIONS

This application is a 35 U.S.C. § 371 national stage application of PCT/US2022/074076 filed Jul. 22, 2022 and entitled “Compositions and Methods for Production of Value-Added Chemicals,” which claims priority to U.S. Provisional Application Ser. No. 63/224,553 filed Jul. 22, 2021 and entitled “COMPOSITIONS AND METHODS FOR PRODUCTION OF VALUE-ADDED CHEMICALS,”, each of which is hereby incorporated herein by reference in its entirety for all purposes.

### REFERENCE TO AN ELECTRONIC SEQUENCE LISTING

The contents of the electronic sequence listing entitled “(3416-07401) Sequence Listing.xml” of size 42,817 bytes and created on Sep. 12, 2022 is incorporated herein by reference in its entirety.

### TECHNICAL FIELD

The present disclosure relates generally to compositions and methods for the production of value-added chemicals. More particularly, the present disclosure relates to chemoenzymatic methods for the production value-added chemicals from biorenewable feedstocks.

### BACKGROUND

Currently, the majority of chemicals and energy are produced from finite fossil fuel-based resources. The world's societies and commercial markets are dependent on these depleting fossil fuels for the production of approximately 80% of its energy and 90% of its chemicals. The large-scale production and use of these fossil fuels have contributed to negatively impacting the environment due to the emission of harmful greenhouse gases and toxic materials. With a growing world population, the demand for energy and chemicals is increasing considerably.

Traditional chemical manufacturing is often based on either (i) a petrochemical pathway using heat to generate a reaction and corresponding products or (ii) a biological pathway typically employing microbes and processes such as fermentation. Both of these routes produce a mixture of products and impurities that require substantial processing in order to obtain the compound(s) of interest at an acceptable percentage purity. As a result, these routes are not very efficient and can be expensive. Further, both of these routes result in the generation of large amounts of CO2, either as a biological byproduct or a product of combustion to generate the heat used for reactions. Typical challenges in the production of value-added chemicals from inexpensive biorenewable feedstocks include the use of reagents and conditions that are harsh, costly, and environmentally unfriendly.

Because of the increasing demands for energy and chemicals and to overcome the issues associated with depleting fossil fuels and its related environmental impact, it is imperative to identify alternate resources to produce energy and chemicals. Biomass is the only viable sustainable feedstock with the potential for carbon-neutral production of commercial chemicals. By leveraging the diversity of functionalities in biomass and the present capabilities of thermo- and biochemical conversion, significant potential exists for bio-based drop-in replacements for petrochemicals as well as new products beyond the scope of the current petrochemical industry.

An ongoing need exists for the elimination and simplification of these expensive and carbon-intensive processing steps typically associated with the commercial production of value-added chemicals.

### BRIEF DESCRIPTION OF DRAWINGS

For a detailed description of the aspects of the disclosed processes and systems, reference will now be made to the accompanying drawings in which:

FIG. 1A is a schematic overview of the processes disclosed herein.

FIG. 1B is a schematic overview of the processes disclosed herein.

FIG. 2 schematically depicts an aspect of the molecular manufacturing processes disclosed herein.

FIG. 3 schematically depicts an aspect of the molecular manufacturing processes disclosed herein.

FIG. 4 schematically depicts an aspect of the molecular manufacturing processes disclosed herein.

FIG. 5 schematically depicts an aspect of the molecular manufacturing processes disclosed herein.

FIG. 6 schematically depicts an aspect of the molecular manufacturing processes disclosed herein.

FIG. 7 schematically depicts an aspect of the molecular manufacturing processes disclosed herein.

FIG. 8 schematically depicts an aspect of the molecular manufacturing processes disclosed herein.

FIG. 9 schematically depicts an aspect of the molecular manufacturing processes disclosed herein.

FIG. 10 schematically depicts an aspect of the molecular manufacturing processes disclosed herein.

FIG. 11 schematically depicts an aspect of the molecular manufacturing processes disclosed herein.

FIG. 12 schematically depicts an aspect of the molecular manufacturing processes disclosed herein.

FIG. 13 schematically depicts an aspect of the molecular manufacturing processes disclosed herein.

FIG. 14 schematically depicts an aspect of the molecular manufacturing processes disclosed herein.

FIG. 15 schematically depicts an aspect of the molecular manufacturing processes disclosed herein.

FIG. 16 schematically depicts an aspect of the molecular manufacturing processes disclosed herein.

FIG. 17 schematically depicts an aspect of the molecular manufacturing processes disclosed herein.

FIG. 18 schematically depicts an aspect of the molecular manufacturing processes disclosed herein.

FIG. 19 schematically depicts an aspect of the molecular manufacturing processes disclosed herein.

FIG. 20 schematically depicts an aspect of the molecular manufacturing processes disclosed herein.

FIG. 21 depicts HPLC traces for the samples from Example 1.

FIG. 22A presents the results of assays of enzymatic activity and reaction schemes for the indicated samples.

FIG. 22B presents the results of assays of enzymatic activity and reaction schemes for the indicated samples.

FIG. 23A presents the results of assays of enzymatic activity and reaction schemes for the indicated samples.

FIG. 23B presents the results of assays of enzymatic activity and reaction schemes for the indicated samples.

FIG. 24 presents the results of assays of enzymatic activity and reaction schemes for the indicated samples.

FIG. 25 presents the results of assays of enzymatic activity and reaction schemes for the indicated samples.

FIG. 26 presents the results of assays of enzymatic activity and reaction schemes for the indicated samples.

FIG. 27A presents the results of assays of enzymatic activity and reaction schemes for the indicated samples.

FIG. 27B presents the results of assays of enzymatic activity and reaction schemes for the indicated samples.

FIG. 28 presents the results of assays of enzymatic activity and reaction schemes for the indicated samples.

FIG. 29A presents the results of assays of enzymatic activity and reaction schemes for the indicated samples.

FIG. 29B presents the results of assays of enzymatic activity and reaction schemes for the indicated samples.

FIG. 30A presents the results of assays of enzymatic activity and reaction schemes for the indicated samples.

FIG. 30B presents the results of assays of enzymatic activity and reaction schemes for the indicated samples.

FIG. 31 presents the results of assays of enzymatic activity and reaction schemes for the indicated samples.

FIG. 32A presents the results of assays of enzymatic activity and reaction schemes for the indicated samples.

FIG. 32B presents the results of assays of enzymatic activity and reaction schemes for the indicated samples.

FIG. 33 presents the results of assays of enzymatic activity and reaction schemes for the indicated samples.

FIG. 34 presents the results of assays of enzymatic activity and reaction schemes for the indicated samples.

FIG. 35 presents the results of assays of enzymatic activity and reaction schemes for the indicated samples.

FIG. 36 schematically depicts an aspect of the molecular manufacturing processes disclosed herein.

FIG. 37 presents the results of assays of enzymatic activity and reaction schemes for the indicated samples.

FIG. 38 schematically depicts an aspect of the molecular manufacturing processes disclosed herein.

FIG. 39 schematically depicts an aspect of the molecular manufacturing processes disclosed herein.

FIG. 40 presents the results of assays of enzymatic activity and reaction schemes for the indicated samples.

FIG. 41 presents the results of assays of enzymatic activity and reaction schemes for the indicated samples.

FIG. 42 presents the results of assays of enzymatic activity and reaction schemes for the indicated samples.

FIG. 43 presents the results of assays of enzymatic activity and reaction schemes for the indicated samples.

FIG. 44 presents the results of assays of enzymatic activity and reaction schemes for the indicated samples.

FIG. 45 presents the results of assays of enzymatic activity and reaction schemes for the indicated samples.

FIG. 46 presents the results of assays of enzymatic activity and reaction schemes for the indicated samples.

FIG. 47 presents the results of assays of enzymatic activity and reaction schemes for the indicated samples.

FIG. 48 schematically depicts an aspect of the molecular manufacturing processes disclosed herein.

FIG. 49 presents the results of assays of enzymatic activity and reaction schemes for the indicated samples.

### DETAILED DESCRIPTION

### Overview

Disclosed herein are methods and compositions for the molecular transformations of feedstocks such as sugars, air, and carbon dioxide to value-added chemicals (VAC). The processes disclosed herein utilize a combination of biocatalysts and metal catalysts to perform a combination of commercially important transformations such as oxidation, dehydration, carboxylation, carboxyligation and hydrogenation. In an aspect of the present disclosure, a platform chemical is used to produce a VAC. In an aspect, the platform chemical comprises alcohols such as ethanol and methanol, sugars such as glucose, hydroxymethylfurfural, 2,5-furandicarboxylic acid, glycerol, ethylene glycol, succinic acid, nicotinamide or combinations thereof.

As disclosed herein, the reaction chemistry comprises four-unit operations, from which 90% of the chemicals, ingredients, and materials that underpin modern society can be manufactured. The methods disclosed herein utilize primarily oxidation, dehydration, carboxylation, and/or hydrogenation reactions for the production of VAC. Accordingly, as disclosed herein, a system using enzymes and catalysts (e.g., a heterogeneous catalyst such as a heterogeneous metallic catalyst) can perform at least one of an oxidation reaction, a dehydration reaction, a carboxylation reaction, or a hydrogenation reaction to produce a product from a reactant. Various separation steps can then be used to isolate the products and recycle unreacted material to the reaction train for continued reaction within the system. Overall, the processes disclosed herein can advantageously generate fewer side products which will positively contribute to the economics of the process.

Within the systems and processes disclosed herein, at least three enzymatic transformations are being developed including, oxidation, dehydration, and carboxylation. These reactions allow the system and methods to transform a variety of different feedstocks and intermediates by directly reacting with an oxygen source (e.g., air) or carbon dioxide to produce intermediates and/or products. In general, the enzymes can be contacted with the reactants in an appropriate environment such as an aqueous environment (e.g., buffer) and contacted with the reactants. The reactants can be heterogeneous (e.g., gas/liquid environments) and/or homogeneous. Within the reaction, the enzymes can produce products in a variety of chemical cycles such that the enzymes can perform the reaction over a commercially suitable time period while maintaining sufficient activity during the reaction cycles. It is contemplated that the biocatalysts of the present disclosure may experience some loss of catalytic or structural integrity over time. In one or more aspects, a method of the present disclosure comprises the regeneration or replacement of all or a portion of the enzyme catalyst.

The selection of the enzymes used within the system is also a component of the methods described herein. In most systems, an organism containing a biocatalyst having one or more characteristics that meet some user and/or process goals can be developed or selected for use within the methods disclosed herein. However, the use of biological organisms can result in the generation of range of reaction products including byproducts and carbon dioxide. Rather than engineer organisms, the presently disclosed methods contemplate the rational engineering of enzymes which can be selected for one or more characteristics that meet some user and/or process goal such as specific reaction rates and/or substrate selectivity.

Any suitable method for enzyme selection may be employed. For example, the enzyme selection and development effort can include high throughput screening along with computational design and machine learning algorithms to achieve a desired result. Once selected, the enzymes can be produced (e.g., via fermentation) with subsequent isolation and the purified enzymes may be deployed within the reaction vessel in the absence of any source organisms.

In one or more aspects, the production of enzyme catalysts uses orthogonal microbial host platforms so that proteins from different sources can be rapidly tested with short design-build-test-analyze cycles and scaled up. Enzyme catalysts used in the processes can be available from a wide variety of natural sources (e.g., plants, bacteria, fungi) and therefore are not always readily expressed in a single host. The presently disclosed methodologies may utilize one or more of the three classes of source organisms are used in the enzyme production process: bacteria, yeast, and fungi, as protein production hosts to yield valuable enzymes that may not be expressed in any singular organism.

State of the art organism engineering capabilities coupled with automation and AI allow for design-build-test-analyze cycles to remain short and allow for continuous improvement through recursive learning all geared towards one goal: protein production. In this way, the highly tailored enzymes generated can be easily scaled up and isolated to be used in the systems and processes disclosed herein.

The catalysts utilized in the present disclosure can be heterogenous metal catalysts in some aspects. Heterogeneous metal catalysts typically consist of small metallic nanoparticles dispersed across a high surface area porous support. The support can have a suitable pore size to allow for the reaction of the various molecules used in the processes. In some aspects, the metal catalysts can operate in an aqueous fluid (e.g., water, buffer) at low temperatures without significantly degrading over time. In some aspects, a catalytic component can include gold, which can allow for oxidation, dehydration, and hydrogenation reactions. Gold-containing catalysts are not useful for petrochemical reactions because they deactivate at high temperatures. In general, the processes and reactions disclosed herein can take place below 300° F., which can allow for reactions with the metallic catalyst and enzymes to perform highly selective molecular transformations at high throughputs and yields.

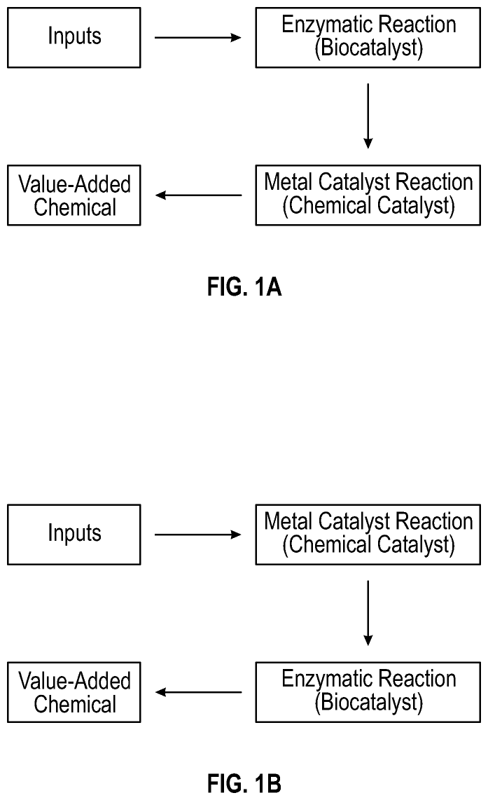

The overall process is shown in FIGS. 1A and 1B. As shown in FIG. 1A, the process includes reacting inputs comprising the reactants in a suitable medium with a biocatalyst (e.g., an enzyme). The output of the enzymatic pathway can produce intermediates which are then reacted using a metal catalyst to produce final products. A separation technique can be utilized at any point in the process to isolate and purify one or more VACs to produce a final product stream.

In some aspects, the order of the reactions can be changed. As shown in FIG. 1B, the process can include reacting inputs comprising reactants with a metallic catalyst in a suitable medium to produce at least one intermediate. The output of the metallic catalyst reactions are intermediates that can then be reacted using a biocatalyst such as an enzyme to produce the final products. A separation technique can be utilized at any point in the process to isolate and purify one or more VACs to produce a final product stream. SUGARS

In an aspect, the platform chemical comprises glucose. In such aspects, the glucose is converted to a VAC such as glucaric acid, D-erythorbic acid, L-ascorbic acid, succinic acid, 2,5-furandicarboxylic acid, or furan dicarboxylic methyl ester.

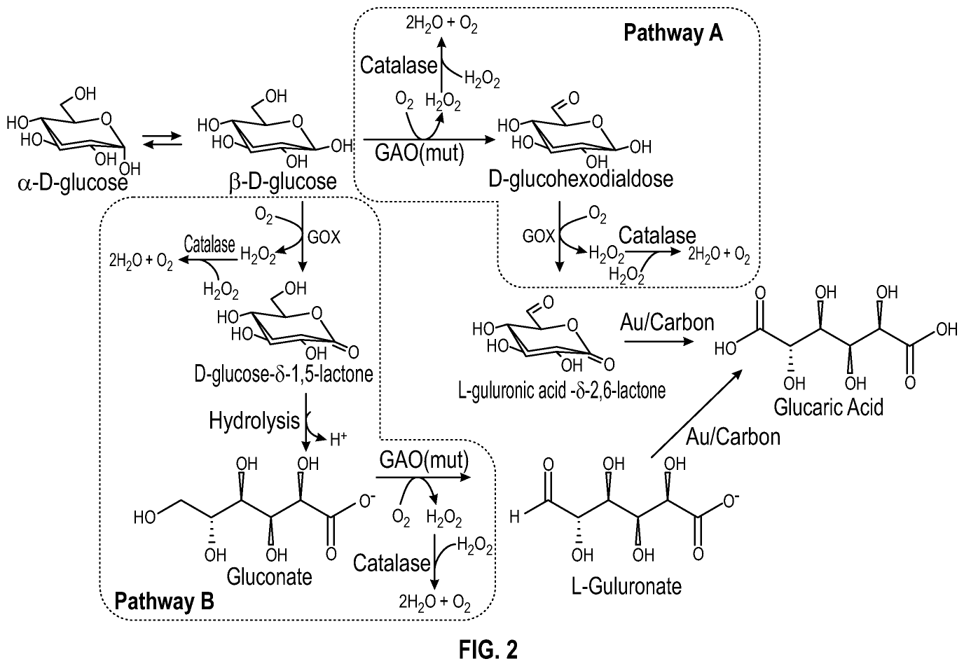

In an aspect, glucose is converted to glucaric acid as depicted schematically in FIG. 2. Referring to FIG. 2, Pathway A, glucose isomerizes between α-D-glucose and β-D-glucose. Glucose may be contacted with a galactose oxidase (GAO) variant under conditions suitable for oxidation of the C₆ alcohol to an aldehyde generating D-glucohexodialdose. D-glucohexodialdose may then be contacted with a glucose oxidase (GOX) under conditions suitable for oxidation of the C₁ alcohol to produce L-guluronic acid-β-2,6-lactone. L-guluronic acid-8-2,6-lactone which is in equilibrium with L-guluronic acid may be harvested directly or further reacted with a heterogeneous metal catalyst (HMC) or transition metal catalyst (TMC) under conditions suitable for the formation of glucaric acid.

In an alternative aspect, depicted as Pathway B in FIG. 2, a GAO variant and GOX are simultaneously contacted with glucose under conditions suitable for the production D-glucono-δ-1,5-lactone. In one or more aspects, D-glucono-δ-1,5-lactone is further processed and isolated as a product. In the alternative, D-glucono-δ-1,5-lactone is acidified to form gluconate which is contacted with a GAO under conditions suitable for the formation of L-guluronate. Acidification may be carried out using any suitable acidizing agent (e.g., HCl). L-guluronate may be contacted with an HMC under conditions suitable for the formation of glucaric acid.

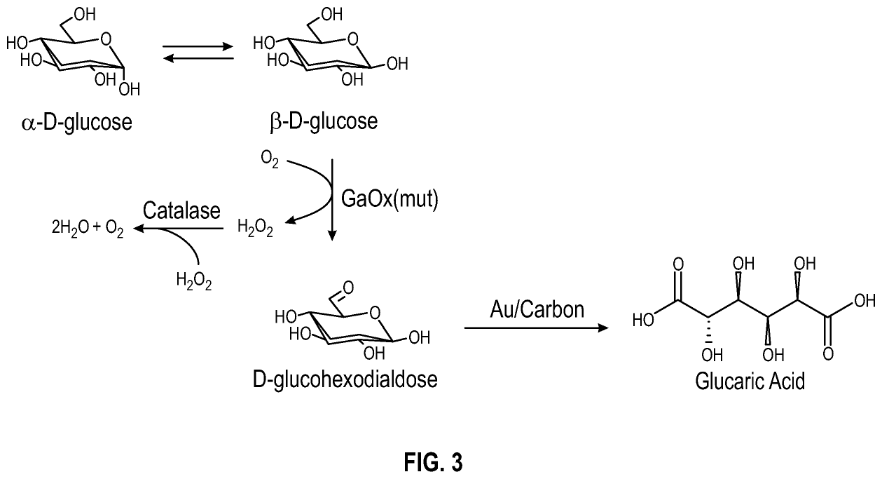

In an alternative aspect, as depicted schematically in FIG. 3, a GAO variant is contacted with glucose under conditions suitable for oxidation of the C₆ alcohol of glucose to an aldehyde generating the dialdehyde, D-glucohexodialdose. D-glucohexodialdose may then be contacted with an HMC and/or TMC under conditions suitable for the formation of glucaric acid.

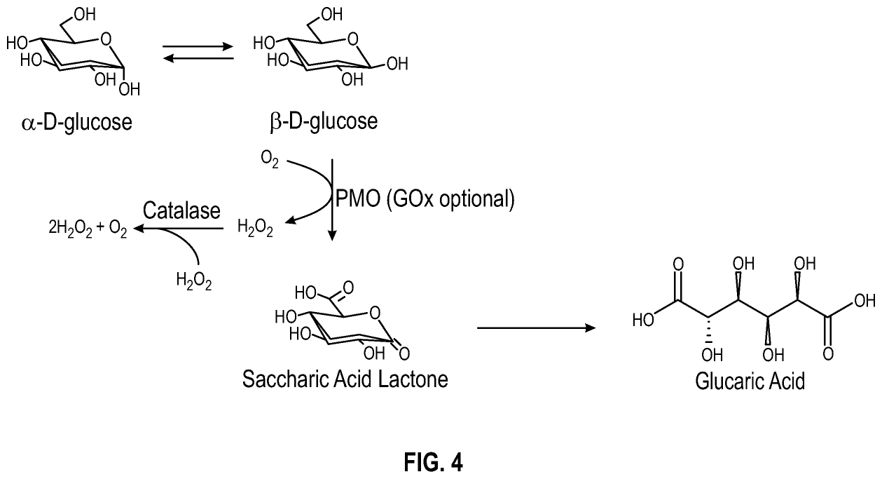

In one or more aspects, a method of producing the VAC glucaric acid comprises contacting, a polysaccharide monooxygenase (PMO) with the platform chemical glucose under conditions suitable for the oxidation of both the C₁ and C₆ alcohols of glucose to form saccharic acid lactone. This reaction is depicted schematically in FIG. 4. The lactone is easily hydrolyzed under alkaline conditions, greater than about pH 7 to form glucaric acid. Notably, saccharic acid lactone will also slowly self-hydrolyze to form the free acid under relevant reaction conditions.

In one or more aspects, PMO may be combined with a GOX to oxidize the C₁ alcohol of glucose. Because PMO is also suspected of oxidizing the C₄ alcohol to a ketone when provided hydrogen peroxide, catalase can be added to limit availability of this oxidizing agent, suppressing the undesirable C₄ keto pathway. Products from this process may also be passed over a HMC to oxidize any unreacted sugars to the diacid.

In one or more aspects, glucose is contacted with an enzymatic oxidizing composition (EOC) comprising a GOX, an animal peroxidase (XPO), halide ions, a nitroxyl radical mediator (NRM) or a combination thereof. Herein a “halide” has its usual meaning; therefore, examples of halides include fluoride, chloride, bromide, and iodide.

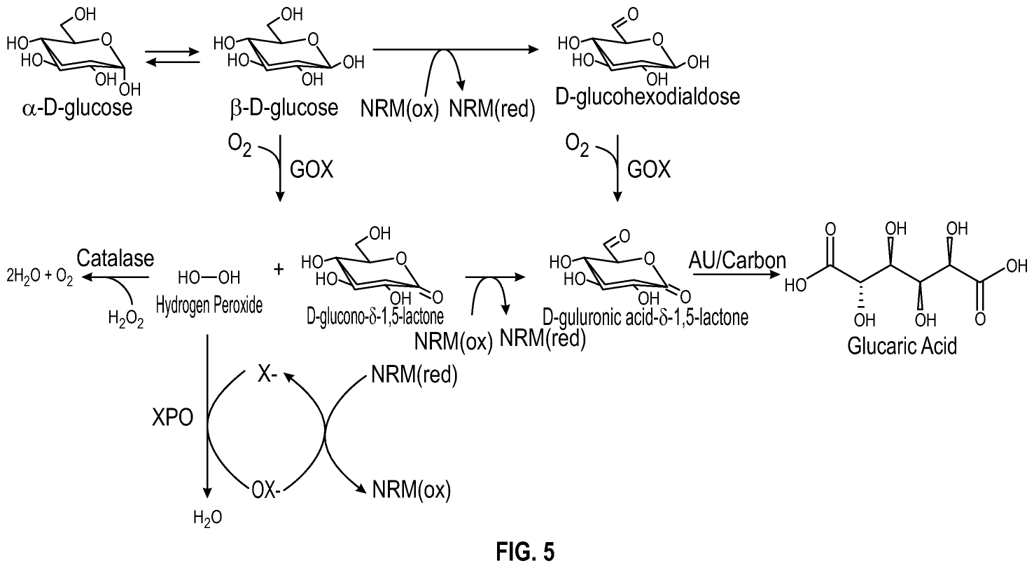

In an aspect and with reference to FIG. 5, glucose may be contacted with an NRM under conditions suitable for the formation of D-glucohexodialdose. D-glucohexodialdose may then be contacted with a GOX under conditions suitable for the formation of D-guluronic acid-δ-1,5-lactone which can be converted to glucaric acid in the presence of a HMC. In the alternative, glucose is contacted first with a GOX under conditions suitable for the formation of D-glucono-δ-1,5-lactone. NRMs may be included in the reaction to promote formation of D-glucuronic acid-δ-1,5-lactone from D-glucono-δ-1,5-lactone and its subsequent oxidation to glucaric acid using an HMC.

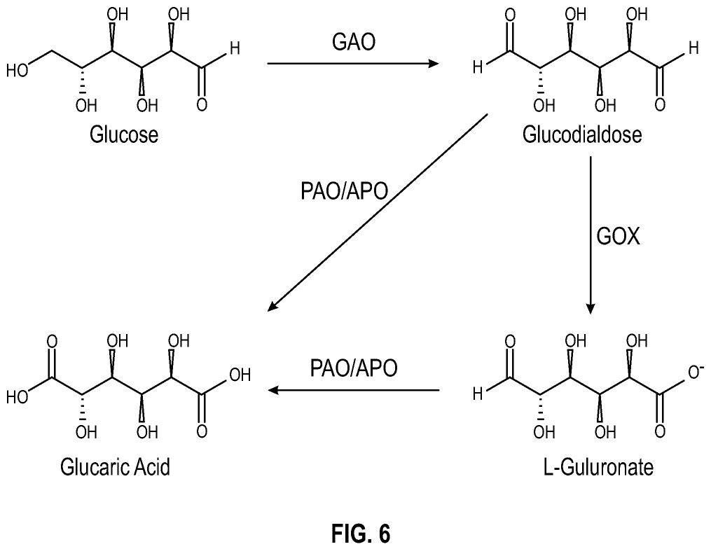

In an aspect and with reference to FIG. 6, glucose may be contacted with a GAO under conditions suitable for the formation of D-glucohexodialdose. D-glucohexodialdose may optionally be contacted with a GAO to generate D-guluronic acid. D-glucohexodialdose or D-guluronic acid may then be contacted with a periplasmic aldehyde oxidase (PAO) or unspecific peroxygenase (UPO) to form glucaric acid.

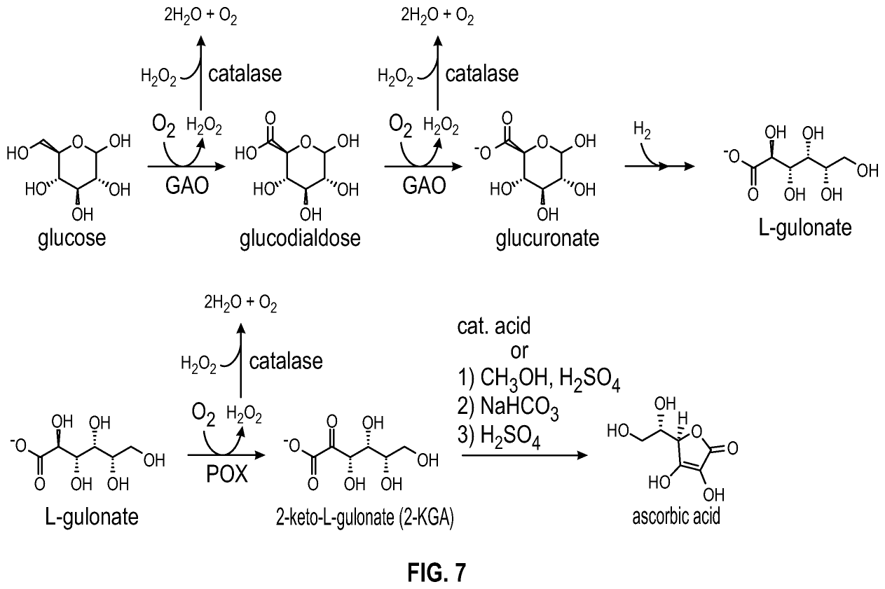

In another aspect, FIG. 7 schematically depicts an aspect of the chemoenzymatic method for production of L-ascorbic acid (L-AA) from glucose. Referring to FIG. 7, an engineered galactose oxidase (GAO) is used to oxidize the glucose to an aldehyde. The aldehyde is then reduced via a metal hydrogenation catalyst to produce D-gluconate. A pyranose-2-oxidase (POX) enzyme is then used to oxidize D-gluconate to 2-KGA, which can be cyclized to form LAA via an acid catalyst, methyl esterification, lactonization, protonation or a combination thereof. Catalase can be added to any step involving an oxidase generating hydrogen peroxide in order to break this reactive chemical into harmless water and oxygen, thereby preserving enzyme function.

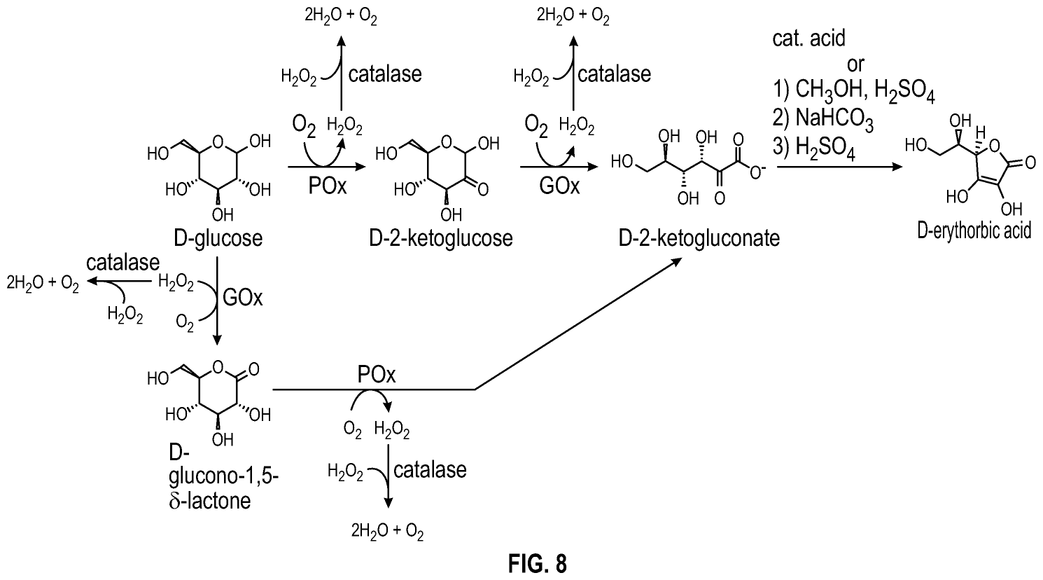

In an aspect and with reference to FIG. 8, GOX oxidizes C₁ in glucose to generate gluconolactone. Simultaneously, POX oxidizes the C₂ to generate 2-ketoglucose. The combination of both enzymes generates 2-ketogluconate. The product (i.e., 2-ketogluconate) may be isolated and subsequently reacted with methanol in the presence of acid to form methyl-2-ketogluconate. Formation of the methyl ester prevents esterification of alcohols and carboxylates upon heating. In one or more aspects, the addition of sodium bicarbonate and sulfuric acid is used to generate sodium methoxide which can then isomerize methyl-2-ketogluconate to D-EA.

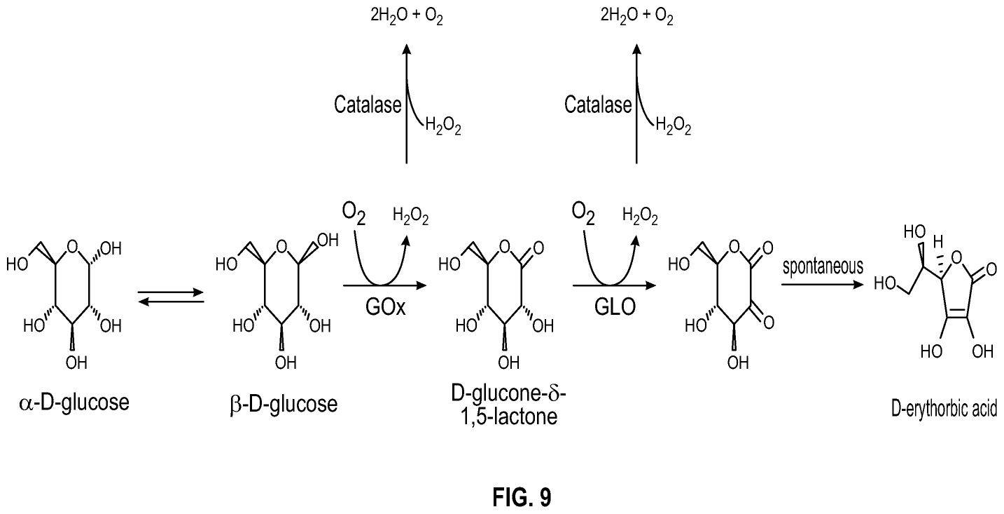

In another aspect of the present disclosure depicted schematically in FIG. 9, D-glucono-1,5-lactone is synthesized from D-glucose via GOX. D-gluconolactone serves as a substrate for GLO which generates D-EA. Catalase may be added to degrade hydrogen peroxide produced in the GOX and GAO reactions to preserve enzyme function. Oxygen is used to drive the oxidation reactions through acting as an electron acceptor, which drives cofactor regeneration.

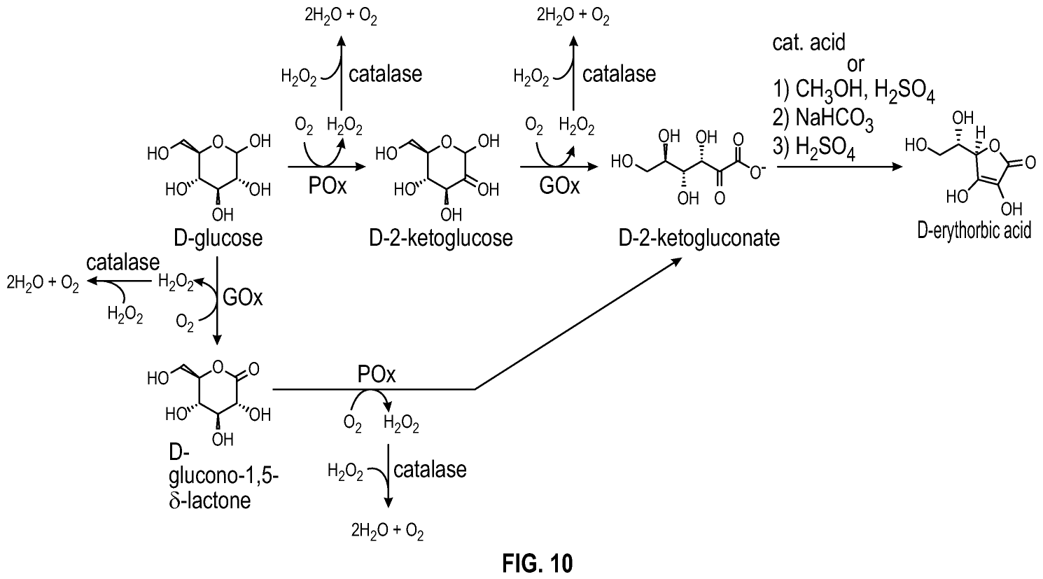

In another aspect of the present disclosure shown in FIG. 10, a mixture of GOX and POX enzymes are deployed sequentially or in combination on a glucose substrate to generate 2-KG, which is then cyclized via acid catalysis. In FIG. 4, POX is used to catalyze the conversion of glucose to 2-ketoglucose. This compound is then oxidized to 2-KG using a heterogeneous metal catalyst and subsequently cyclized either in an acid-catalyzed process or by methyl esterification, lactonization, and protonation, similar to the Reichstein process. In an aspect, a metal catalyst is used to generate 2-KG as this eliminates the need to add stoichiometric quantities of base (i.e., sodium hydroxide) to stabilize pH and maintain function of the POX enzyme.

In an aspect, the metal catalyst comprises a metal oxidation catalyst. In such aspects, the metal oxidation catalyst is a supported transition-metal oxidation catalyst, alternatively a nanoparticle supported transition-metal oxidation catalyst. Hereinafter, these are collectively designated as the “TMC.” In an aspect, the support comprises carbon, silica, alumina, titania (TiO₂), zirconia (ZrO₂), a zeolite, or any combination thereof, which contains less than about 1.0 weight percent (wt. %), alternatively less than about 0.1 wt. %, or alternatively less than about 0.01 wt. % SiO2 binders based on the total weight of the support.

Suitable support materials are predominantly mesoporous or macroporous, and substantially free from micropores. For example, the support may comprise less than about 20% micropores. In an aspect, the support is a porous nanoparticle support. As used herein, the term “micropore” refers to pores with diameter <2 nm, as measured by nitrogen adsorption and mercury porosimetry methods and as defined by IUPAC. As used herein, the term “mesopore” refers to pores with diameter from ca. 2 nm to ca. 50 nm, as measured by nitrogen adsorption and mercury porosimetry methods and as defined by IUPAC. As used herein, the term “macropore” refers to pores with diameters larger than 50 nm, as measured by nitrogen adsorption and mercury porosimetry methods and as defined by IUPAC.

In an aspect, the support comprises a mesoporous carbon extrudate having a mean pore diameter ranging from about 10 nm to about 100 nm and a surface area greater than about 20 m² g⁻¹ but less than about 300 m² g⁻¹. Supports suitable for use in the present disclosure may have any suitable shape. For example, the support may be shaped into 0.8-3 mm trilobes, quadralobes, or pellet extrudates. Such shaped supports enable the use of fixed trickle bed reactors to perform the final oxidation step under continuous flow.

In one or more aspects, the metal comprises a Group 8 metal (e.g., Re, Os, Ir, Pt, Ru, Rh, Pd, Ag), a 3d transition metal, an early transition metal, or a combination thereof. In an aspect, the TMC comprises gold, Au.

In an aspect, the TMC comprise platinum and gold, and are heterogeneous, solid-phase TMCs. In such aspects, suitable catalyst supports include, without limitation, carbon, surface treated aluminas (such as passivated aluminas or coated aluminas), silicas, titanias, zirconias, zeolites, montmorillonites, and modifications, and mixtures or combinations thereof. The catalyst support may be treated so as to promote the preferential deposition of platinum and gold on the outer surface of the support so as to create a shell type TMC. The platinum and gold-containing compounds that function as a TMC may be produced by any suitable methodology. For example, the platinum and gold-containing TMCs may be produced using deposition procedures such as incipient wetness, ion-exchange, and deposition-precipitation.

In other aspects, the metal catalyst is a TMC comprising metal phases that are monometallic or multimetallic combinations of Cu, Ag, Au, Ni, Pd, Pt, or Ir. The activity, selectivity, and stability of the active phases can be modulated with dopants of early 3d, 4d, and 5d transition metals, or heavy post transition metals such as Sn, Sb, and Bi. In some aspects, metals (e.g., Group 1 metals) are intercalated into the metal lattice to modulate catalyst properties. In an aspect, salt precursors of the active phases are deposited onto a support of the type disclosed herein using any suitable methodology. For example, deposition of the active phases may be carried out using techniques such as incipient wetness impregnation, bulk adsorption impregnation, or deposition precipitation.

In an aspect, the deposited salt precursor of the active phase is then converted to the active phase via Liquid Phase Reduction (LPR) with a suitable salt (e.g., formate salt) at temperatures of less than about 100° C. or via Gas Phase Reduction (GPR) at temperatures ranging from about 200° C. to about 500° C. or alternatively from about 200° C. to about 450° C. In an aspect, the metal catalyst comprises gold, platinum or a combination thereof and calcination in air at temperatures of equal to or greater than about 150° C. is performed.

In an aspect, the amount of active phase loaded onto a support of the type disclosed herein is less than about 2.0 weight percent (wt. %), alternatively less than about 1.5 wt. %, or alternatively less than about 1.0 wt. % based on the total weight of the TMC metal catalyst. In an aspect, the amount of active phase loaded onto a support of the type disclosed herein is equal to or less than about 0.5 wt. % based on the total weight of the TMC metal catalyst. In an aspect, the radial distribution of the active phase across the support is anisotropic where the active phase is substantially concentrated in a <500 μm annulus near the surface of the extrudate support in a “core-shell” configuration. A TMC metal catalyst of the type disclosed herein may be characterized by a productivity for the conversion of aldehyde functionalities to carboxylic acids of equal to or greater than about 0.05 mol acid g⁻1 active metal h⁻1 or equal to or greater than about 0.1 mol acid g⁻1 active metal h⁻1 at selectivities from about 70% to about 90%, alternatively equal to or greater than about 70%, alternatively equal to or greater than about 80%, alternatively equal to or greater than about 85%, or alternatively equal to or greater than about 90%. In such aspects, the TMC metal catalyst exhibits conversions of from about 60% to about 95%, alternatively equal to or greater than about 70%, alternatively equal to or greater than about 80%, or alternatively equal to or greater than about 90%. Such TMC metal catalysts may display a steady state leaching amount of from about 1 ppb to about 100 ppb, alternatively less than about 100 ppb, or alternatively less than about 90 ppb. In an aspect, a TMC metal catalyst of the type disclosed herein may be utilized in a temperature range of from about 40° C. to about 120° C., alternatively form about 40° C. to about 110° C., or alternatively from about 50° C. to about 100° C. at pressures ranging from about 10 bar to about 100 bar, alternatively from about 20 bar to about 100 bar, or alternatively from about 20 bar to about 90 bar.

In an aspect, a small molecule chemical catalyst is used in reactions of the present disclosure such as an acid or base. Examples of acids or bases suitable for use as a finishing catalyst include without limitation hydrochloric acid, sulfuric acid, formic acid, sodium hydroxide, and urea.

In an aspect, a reaction mixture for the production of D-EA in addition to one or more biocatalysts may further include one or more purified enzyme cofactors. Nonlimiting examples of purified enzyme cofactors suitable for use in the present disclosure include thiamine pyrophosphate, NAD⁺. NADP⁺, pyridoxal phosphate, methyl cobalamin, cobalamine, biotin, Coenzyme A, tetrahydrofolic acid, menaquinone, ascorbic acid, flavin mononucleotide, flavin adenine dinucleotide, and Coenzyme F420. Such cofactors may be included in the initial reaction mixture and/or be added at various points during the reaction. In some aspects, cofactors may be readily regenerated with oxygen and/or may remain stable throughout the lifetime of the biocatalyst(s).

In one or more aspects, hydrogen peroxide generated during the oxidation reactions disclosed herein may be disproportionated using any suitable method. For example, the hydrogen peroxide may be disproportionated by contact with a catalase, under suitable conditions, to form water and molecular oxygen. In such aspects, molecular oxygen may be recovered as a product that is recycled and used a component in additional reactions.

In an aspect, the VAC succinic acid is synthesized from the platform chemical glucose. In such aspects, a process of the present disclosure comprises oxidation of glucose to form a 2-keto intermediate. In an aspect, the oxidation of glucose is catalyzed by a TMC. Any TMC capable of oxidizing glucose to produce a 2-keto intermediate may be employed. In an aspect, the TMC comprises platinum, bismuth, gold, or a combination thereof. In such aspects, the TMC may be supported on materials such as carbon or alumina. In an aspect, the TMC is a supported Pt/Bi/Au catalyst. In an aspect of the present disclosure, the contacting of glucose with a supported-Pt/Bi/Au catalyst under suitable conditions results in the formation of the 2-keto intermediate, 2-keto-gluconic acid.

In an alternative aspect for the production of succinic acid, the catalyst is an enzyme, alternatively alcohol oxidase (AOX, E.C. 1.1.3.13) or an alcohol oxidase homolog. AOX is a ubiquitous flavin-dependent enzyme that oxidizes lower primary alcohols to aldehydes using oxygen as an oxidizing agent. AOX may be sourced from methylotrophic yeast of the species Kloeckera, Torulopsis, Candida, Pichia, Hanseniaspora, and Metschnikowia. In an alternative aspect, the AOX is sourced from methanol-utilizing bacteria such as Methylococcus capsulatus, thermophilic soil fungi such as Thermoascus aurantiacus, and brown rot fungus such as Gloeophyllum trabeum. Alternatively, the AOX may be sourced from the white-rot basidiomycete Phanerochaete chrysosporium. Contacting of glucose with an AOX under suitable conditions results in the formation of the 2-keto intermediate, 2-keto-gluconate.

In yet another aspect, a method of converting glucose to succinic acid comprises decarboxylation of the 2-keto intermediate to form D-ribulose. In an aspect, decarboxylation of the 2-keto intermediate is carried out in the presence of any suitable catalyst. For example, the decarboxylation may be carried out in the presence of copper ions in association with a polymer matrix. In an aspect, the catalyst comprises CuSO₄ and polyvinylpyrrolidone, and the reaction is carried out in an oxidizing atmosphere.

In an aspect, the methods of converting glucose to succinic acid further comprise dehydration of D-ribulose to generate furfural. Dehydration of D-ribulose may be carried out under suitable conditions in the presence of an acid catalyst. An acid catalyst suitable for use in the present disclosure is a solid acid catalyst, alternatively an ion-exchange resin acid catalyst. For example, the acid catalyst may comprise AMBERLYST™ 15DRY Polymeric Catalyst, which is a bead-form, strongly acidic catalyst. In an alternative aspect, the dehydration of D-ribulose is carried in the presence of formic acid and AMBERLYST™ 15DRY Polymeric Catalyst. In such aspects, the formic acid may be removed with the addition of an oxidant such as hydrogen peroxide.

In an aspect, the methods of converting glucose to succinic acid further comprise oxidation of furfural to generate succinic acid. In an aspect, oxidation of furfural to succinic acid is carried out in the presence of an oxidant and a catalyst. For example, the acid catalyst may comprise AMBERLYST™ 15DRY Polymeric Catalyst and the oxidant may comprise hydrogen peroxide. The reaction may be carried out under conditions suitable for the conversion of furfural to succinic acid.

In an aspect, the VAC 2,5-furan dicarboxylic acid (FDCA) or (FDME) is generated from the platform chemical glucose. In such aspects, glucose may be contacted with an enzyme, (e.g., GAO), a base (e.g., NaOH) and air under conditions suitable for the formation of an oxidized product.

### Alcohols

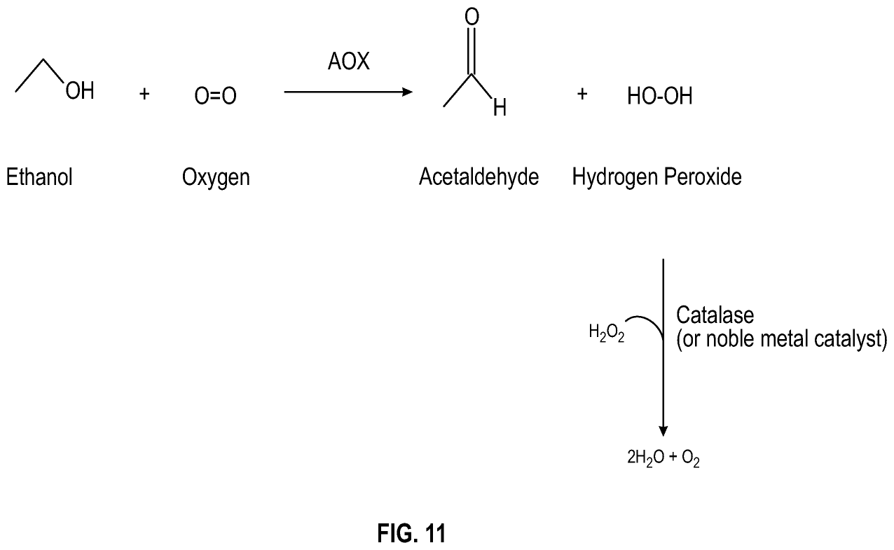

In an aspect, the platform chemical is an alcohol such as ethanol. In an aspect, a method of the present disclosure comprises contacting the ethanol with an alcohol oxidase (E.C. 1.1.3.13) under conditions suitable for the formation of acetaldehyde. This is depicted schematically in FIG. 11.

In an aspect, a method of the present disclosure comprises production of the VAC propylene glycol from the platform chemical ethanol. In such aspects, ethanol is contacted with an ethanol oxidase of the type disclosed herein (e.g., AOX, GAO) under conditions suitable for the formation of an aldehyde. The method may further comprise contacting of the aldehyde with a biocatalyst under conditions suitable for the formation of pyruvic acid. Any biocatalyst suitable for the conversion of an aldehyde to pyruvic acid may be employed. In an aspect, the aldehyde is contacted with a pyruvate decarboxylase (PDC) in the presence of carbon dioxide under conditions suitable for the formation of pyruvic acid. In an aspect, a method of the present disclosure further comprises hydrogenation of the pyruvate in the presence of a hydrogenation catalyst under conditions suitable for the formation of propylene glycol.

In an aspect, disclosed herein are methods for the production of the VACs lactic acid, acrylic acid, propylene glycol, and propanol from the platform chemical ethanol. In an aspect, the methods disclosed herein involve the chemoenzymatic conversion of ethanol to lactic acid, acrylic acid, propylene glycol, or propanol. Hereinafter, lactic acid, acrylic acid, propylene glycol and propanol may be referred to individually as a “C3 product,” or collectively as “C3 products.”

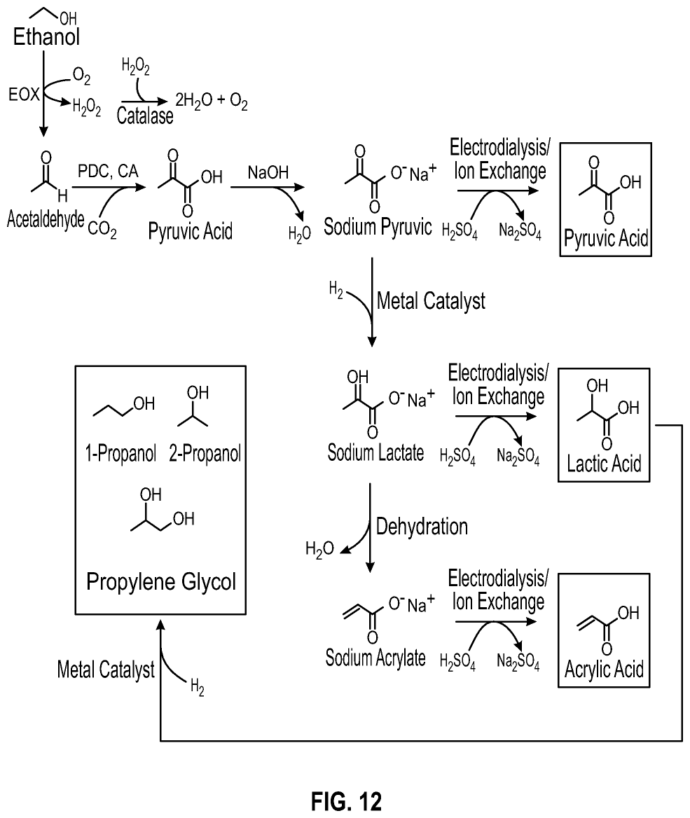

In an aspect, methods for the production of a C3 product comprise the contacting of ethanol with one or more enzymes under conditions suitable to produce an intermediate (e.g., acetaldehyde). This is depicted in FIG. 12.

Referring to FIG. 12, a method of the present disclosure comprises a Stage I where ethanol is contacted with an enzyme capable of catalyzing the selective aerobic oxidation of ethanol to acetaldehyde. In an aspect, this enzyme is an ethanol oxidase (EOX). Stage 1 may further comprise contacting of the aldehyde with a pyruvate decarboxylase (PDC) in the presence of carbon dioxide (CO₂) or a source of CO₂ to generate pyruvic acid. Equilibrium between pyruvic acid and pyruvate may be established using any suitable methodology such as pH adjustment, electrolysis or ion exchange, as depicted in FIG. 12. In an aspect, the pH adjustment is through addition of a base comprising a monovalent cation. For example, the base may be sodium hydroxide, which forms a cation pair comprising a single pyruvate anion and a sodium cation.

The method may further comprise a Stage 2 where pyruvate generated from the reaction of ethanol and EOX is subsequently converted to lactate by partial hydrogenation followed by dehydration to acrylic acid. Alternatively, pyruvate or lactate may be partially hydrogenated to 1,2-propanediol (propylene glycol) or n-propanol.

The Stage 2 portion of methods disclosed herein can alternatively proceed via two routes; both catalyzed by a metal catalyst. The first route uses a caustic hydroxide coupled with electrodialysis and ion exchange to co-produce sodium sulfate or gypsum. The second route uses ammonia and esterification to proceed via ethyl ester intermediates (e.g. ethyl pyruvate, ethyl lactate, and ethyl acrylate) without the co-production of salts.

In an aspect, the hydrogenation catalyst comprises a metal catalyst, alternatively a supported metal catalyst. Equilibrium between lactate and lactic acid may be established using any suitable methodology such as pH adjustment, electrolysis or ion exchange. In some aspects, the metal catalyst is chiral and catalyzes the production of predominately or exclusively R-lactic acid; alternatively, predominately or exclusively S-lactic acid.

In an alternative aspect, Stage 2 further comprises dehydration of lactate to form acrylate. Equilibrium between acrylate and acrylic acid may be established using any suitable methodology such as pH adjustment, electrolysis or ion exchange. In some aspects, dehydration of lactate is catalyzed by a metal catalyst, alternatively a supported metal catalyst. The metal catalyst or supported metal catalyst may be the same as or different from the catalyst utilized to facilitate the reduction of pyruvate. In another alternative aspect, Stage 2 comprises hydrogenation of lactic acid in the presence of a metal catalyst or supported metal catalyst and hydrogen to form a compound containing a propyl group. In an aspect, the compound containing a propyl group comprises 1-propanol, 2-propanol, propylene glycol, or combinations thereof. The metal catalyst or supported metal catalyst may be the same as or different from the catalyst utilized to facilitate the reduction of pyruvate via hydrogenation.

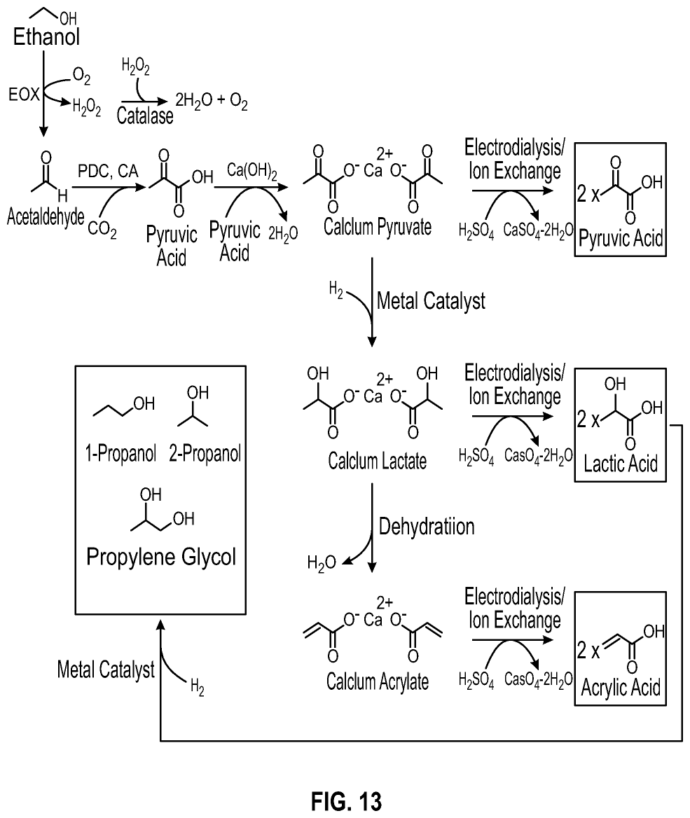

In an aspect, another method for the conversion of ethanol to a C₃ compound is generally depicted in FIG. 13. Referring to FIG. 13, a method of the present disclosure comprises a Stage I where ethanol is contacted with an enzyme capable of catalyzing the selective aerobic oxidation of ethanol to acetaldehyde. In an aspect, this enzyme is an ethanol oxidase (EOX). Stage 1 may further comprise contacting of acetaldehyde with a pyruvate decarboxylase (PDC) in the presence of carbon dioxide (CO₂) or a source of CO₂ to generate pyruvic acid. Equilibrium between pyruvic acid and pyruvate may be established using any suitable methodology such as pH adjustment, electrolysis or ion exchange, as depicted in FIG. 13. In an aspect, pH adjustment occurs by addition of a base comprising a divalent cation. For example, the base may be calcium hydroxide, which is able to provide two hydroxide ions per calcium atom. The addition of calcium hydroxide to the pyruvic acid results in a cation pair comprising two pyruvate anions and a calcium cation.

The method may further comprise a Stage 2 that includes the reduction of pyruvate catalyzed by a hydrogenation catalyst to form lactate. In an aspect, the hydrogenation catalyst comprises a metal catalyst, alternatively a supported metal catalyst. Equilibrium between calcium lactate (having 2 lactate moieties) and lactic acid may be established using any suitable methodology such as pH adjustment, electrolysis or ion exchange, as depicted in FIG. 13. In some aspects, the catalyst is chiral and catalyzes the production of predominately or exclusively R-lactic acid: alternatively, predominately or exclusively S-lactic acid.

In an alternative aspect, Stage 2 further comprises dehydration of lactate to form acrylate. Equilibrium between calcium acrylate (having 2 acrylate moieties) and acrylic acid may be established using any suitable methodology such as pH adjustment, electrolysis or ion exchange, as depicted in FIG. 13. In some aspects, dehydration of lactate is catalyzed by a metal catalyst, alternatively a supported metal catalyst. The metal catalyst or supported metal catalyst may be the same as or different from the catalyst utilized to facilitate the reduction of pyruvate. In another alternative aspect, Stage 2 comprises hydrogenation of lactic acid in the presence of a metal catalyst or supported metal catalyst and hydrogen to form a compound containing a propyl group. In an aspect, the compound containing a propyl group comprises 1-propanol, 2-propanol, propylene glycol, or combinations thereof.

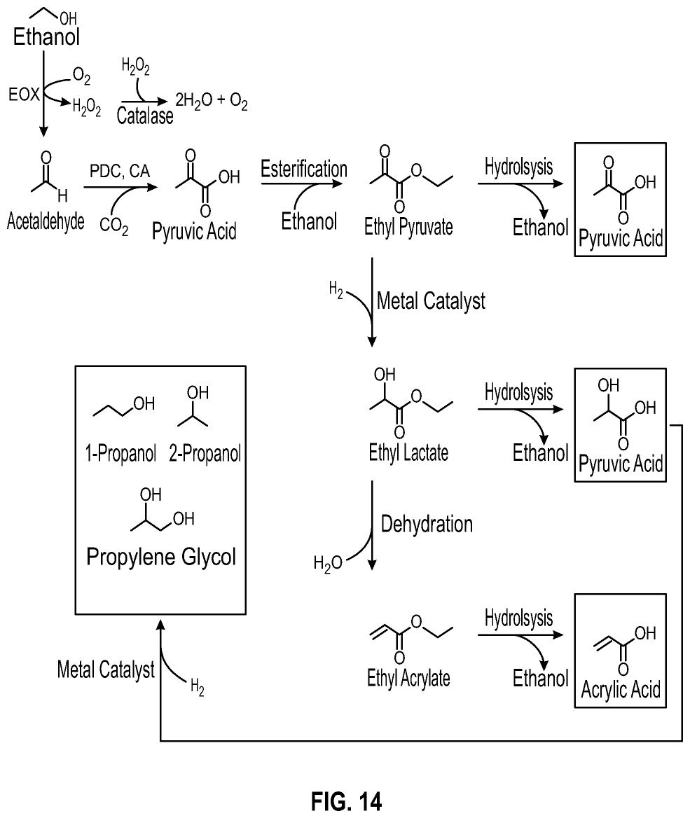

In an aspect, a method for the conversion of the platform chemical ethanol to the value-added C3 compound is generally depicted in FIG. 14. Referring to FIG. 14, a method of the present disclosure comprises a Stage I where ethanol is contacted with an enzyme capable of catalyzing the selective aerobic oxidation of ethanol to acetaldehyde. In an aspect, this enzyme is an ethanol oxidase, EOX. Stage 1 may further comprise contacting of acetaldehyde with a pyruvate decarboxylase (PDC) in the presence of carbon dioxide (CO₂) or a source of CO₂ to generate pyruvic acid. Ethyl pyruvate may be generated through esterification of pyruvate in the presence of ethanol or an organic solvent. Esterification may be achieved using any suitable enzyme such as a CalB lipase (i.e., Novozymes 435 immobilized CalB). Because esterification using lipases typically requires anhydrous conditions, this reaction may occur in the presence of tert-butyl alcohol, methyl butyrate, or other solvent, potentially in a biphasic system.

Ethyl pyruvate may be converted to pyruvic acid by hydrolysis of the ethyl pyruvate, see FIG. 14. The method may further comprise a Stage 2 which includes the reduction of ethyl pyruvate catalyzed by a hydrogenation catalyst to form ethyl lactate. In an aspect, the hydrogenation catalyst comprises a metal catalyst, alternatively a supported metal catalyst. Ethyl lactate may be converted to lactic acid by hydrolysis of the ethyl lactate as depicted in FIG. 14. In some aspects, the catalyst is chiral and catalyzes the production of predominately or exclusively R-lactic acid: alternatively, predominately or exclusively S-lactic acid.

In an alternative aspect, Stage 2 further comprises dehydration of ethyl lactate to form ethyl acrylate. In yet another aspect, ethyl lactate may be converted to lactic acid by hydrolysis. In some aspects, the hydrolysis of ethyl lactate is catalyzed by a metal catalyst, alternatively a supported metal catalyst. The metal catalyst or supported metal catalyst may be the same as or different from the catalyst utilized to facilitate the reduction of pyruvate.

In yet another alternative aspect, Stage 2 comprises hydrogenation of lactic acid in the presence of a metal catalyst or supported metal catalyst and hydrogen to form a compound containing a propyl group. In an aspect, the compound containing a propyl group comprises 1-propanol, 2-propanol, propylene glycol or combinations thereof. The metal catalyst or supported metal catalyst may be the same as or different from the catalyst utilized to facilitate the reduction of pyruvate via hydrogenation.

In an aspect, acetoin, 2,3-butanediol, 1,3-butadiene, and 2-butanone are produced from ethanol. In an aspect, the methods disclosed herein involve the chemoenzymatic conversion of the platform chemical ethanol to acetaldehyde, which is subsequently converted to the VACs acetoin, 2,3-butanediol, 1,3-butadiene, and 2-butanone. Hereinafter, acetoin, 2,3-butanediol, 1,3-butadiene, and 2-butanone may be referred to individually as a “C4 product.” or collectively as “C4 products.”

In an aspect, methods for the production of a C₄ product comprise the contacting of ethanol with one or more enzymes under conditions suitable to produce acetaldehyde. Although ethanol is depicted and discussed herein as the substrate, it is contemplated other substrates may be employed.

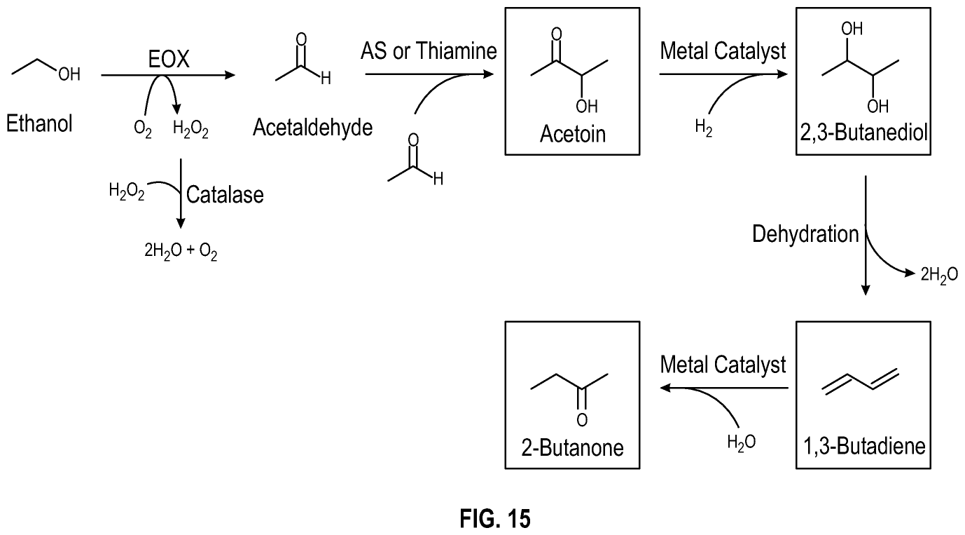

In an aspect, a method for the conversion of ethanol to a C₄ compound is generally depicted in FIG. 15. Referring to FIG. 15, a Stage 1 of the present disclosure comprises conversion of ethanol to acetoin. In an aspect, a method for the conversion of ethanol to a C₄ compound comprises contacting of ethanol with an enzyme capable of catalyzing the selective aerobic oxidation of ethanol to acetaldehyde. In an aspect, this enzyme is an ethanol oxidase, EOX. Stage 1 may further comprise carboligation of two molecules of acetaldehyde to produce acetoin. Any suitable catalyst may be utilized to carry out the carboligation of acetaldehyde molecules. In an aspect, carboligation of the acetaldehyde is catalyzed by an acetoin synthase (AS). In an alternative aspect, carboligation of acetaldehyde is catalyzed by a thiamine-containing catalyst, carbene catalysts such as N-heterocyclic catalysts or combinations thereof. In some aspects, carboligation of acetaldehyde to produce acetoin is carried under conditions suitable to increase the stereospecificity of the reaction product; for example, the reaction product may comprise an excess of or exclusively a particular enantiomer, such as (R)-acetoin. In an alternative aspect, carboligation of acetaldehyde to produce acetoin is carried out under conditions suitable to produce a racemic product. In such aspects, carboligation is catalyzed by enzymes such as formolase (FLS) or pyruvate decarboxylase (PDC).

In an aspect, Stage 2 of the presently disclosed method for the conversion of the platform chemical ethanol to a VAC C₄ compound comprises reduction of acetoin through hydrogenation over a metal catalyst to form 2,3-butanediol. In an aspect, butadiene may be formed by the dehydration of 2,3-butanediol. A method for the conversion of ethanol to a C₄ compound may further comprise partial dehydration of 2,3-butanediol to form 2-butanone. Dehydration of 2,3-butanediol may be catalyzed by a metal catalyst that is the same as or different from the metal catalyst utilized to catalyze the hydrolysis of acetoin.

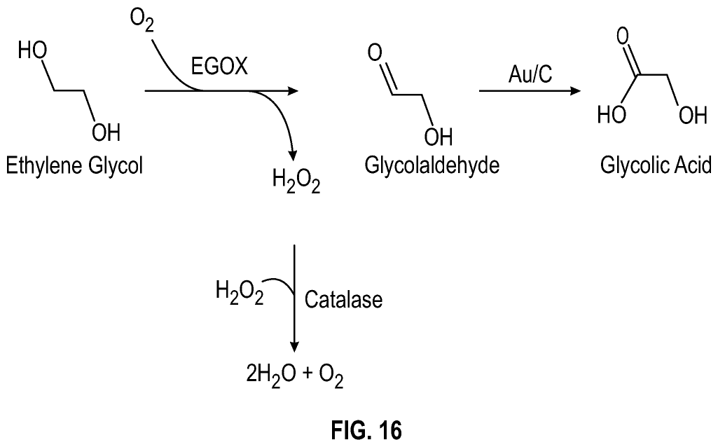

In an aspect, a method for the production of the VAC glycolic acid comprises contacting of the platform chemical ethylene glycol with one or more biocatalysts under conditions suitable to produce glycolaldehyde. Glycolaldehyde may be further contacted with one or more metal catalysts under conditions suitable to produce glycolic acid. The chemoenzymatic method of this disclosure is depicted in FIG. 16.

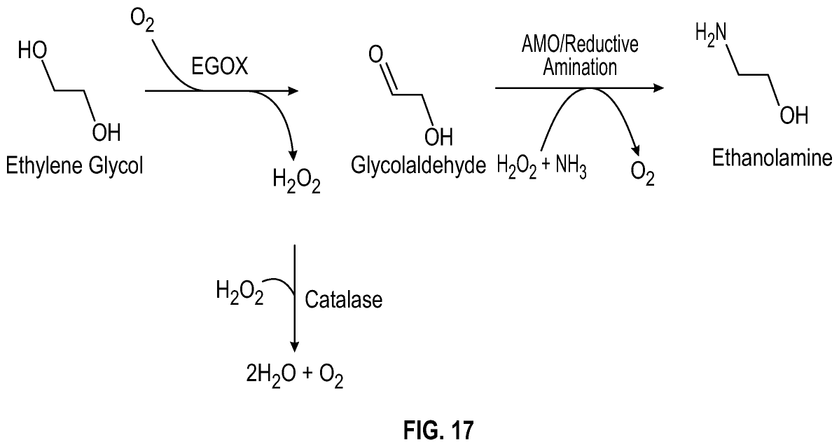

In an aspect, a method for the production the VAC ethanolamine from the platform chemical ethylene glycol is schematized in FIG. 17. In an aspect, a process comprises contacting ethylene glycol with a biocatalyst under conditions suitable for conversion to an oxidized intermediate. In an aspect, the oxidized intermediate is glycolaldehyde. The method may further comprise conversion of glycolaldehyde to EA. The conversion of glycolaldehyde to EA may be catalyzed using a chemical agent or a biocatalyst.

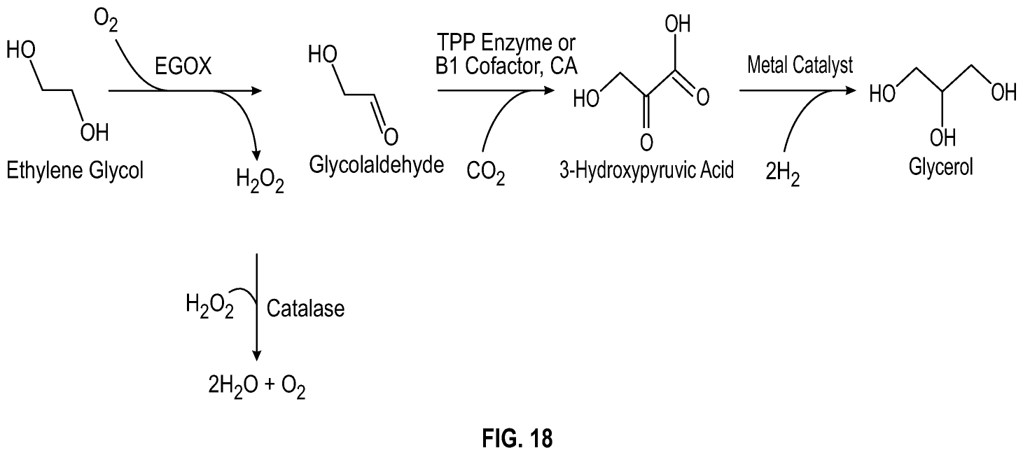

In an aspect, a method for the production of the VAC glycerol from the platform chemical ethylene glycol is schematized in FIG. 18. Referring to FIG. 18, Stage 1 of the present disclosure comprises conversion of ethylene glycol to glycolaldehyde. In an aspect, an enzyme capable of selectively oxidizing ethylene glycol to glycolaldehyde (EGOX) using molecular oxygen and generating hydrogen peroxide is utilized to catalyze the first step. As depicted in FIG. 16, glycolaldehyde and carbon dioxide may be converted to 3-hydroxypyruvic acid using a suitable carboligation catalyst. In an aspect, Stage 2 of the presently disclosed method comprises reduction of 3-hydroxypyruvic acid through hydrogenation over a metal catalyst to form glycerol.

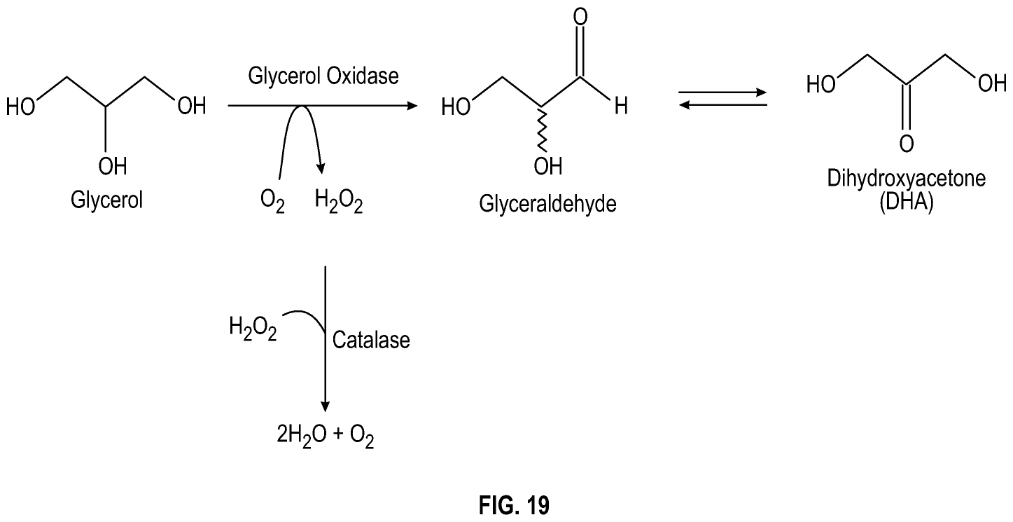

In an aspect, a method for the production of the VAC dihydroxyacetone from the platform chemical glycerol is schematized in FIG. 19. In an aspect, a process comprises contacting glycerol with a biocatalyst under conditions suitable for conversion to an oxidized intermediate. Biocatalysts suitable for use in the production of DHA the biocatalyst comprises an alcohol oxidase (AOX), an alditol oxidase (AIDO), a copper-radical oxidase (CRO), a glycerol oxidase (GlyOX), or combinations thereof. In an aspect, the oxidized intermediate is glyceraldehyde. The method may further comprise conversion of glyceraldehyde to DHA.

### Nicotinamide

In an aspect, an in-vitro chemoenzymatic route to produce the VAC nicotinamide from the platform chemical 3-cyanopyridine is. In such an aspect, 3-cyanopyridine is reacted with water in the presence of a nitrile hydratase under conditions suitable for the formation of nicotinamide. In some aspects, as a result of this process being conducted in vitro, the reaction can occur in water and nicotinamide can be easily recovered at high purity.

In an aspect, hydrolysis of 3-cyanopyridine is catalyzed by any suitable biocatalyst able to facilitate the hydrolysis of a nitrile. In an aspect, the biocatalyst is a nitrile hydratase (NHase).

### HMF

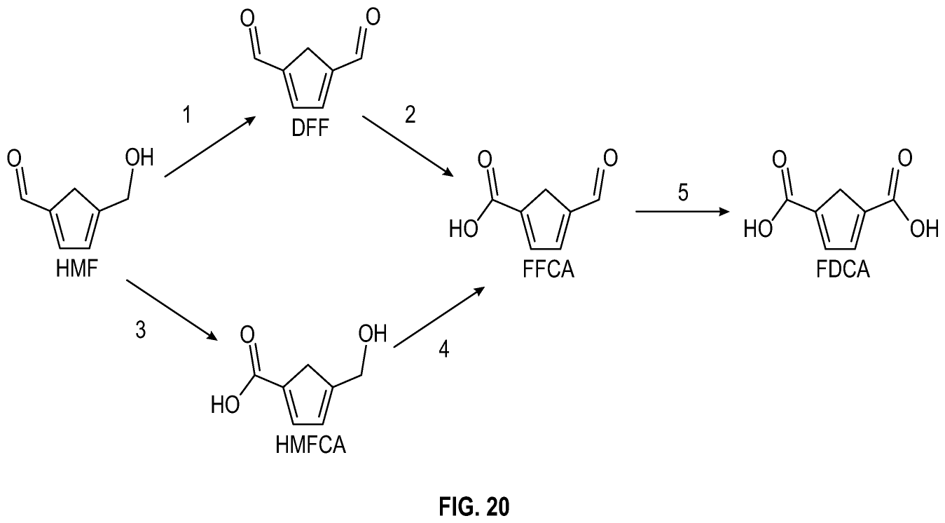

In an aspect, an in-vitro chemoenzymatic route to produce the VAC FDCA from the platform chemical 5-hydroxymethylfurfural (HMF) is depicted in FIG. 20. Disclosed herein are chemoenzymatic methods for the production of FDCA. In an aspect, a method of the present disclosure comprises enzymatic oxidation of HMF, under mild reaction conditions, to produce an intermediate. It is to be understood that HMF is an exemplary reactant for the production of FDCA and other reactants are contemplated by this disclosure. In an aspect, a method of the present disclosure further comprises oxidation of the intermediate by a metal catalyst, alternatively a TMC to produce the FDCA. Oxidative conversion of HMF to FDCA is depicted generally in FIG. 20.

### OXIDATION BIOCATALYST

### Alcohol Oxidase

Exemplary biocatalysts suitable for use in the disclosed processes include but are not limited to alcohol oxidase, galactose oxidase and glycerol oxidase. In an aspect, the oxidation biocatalyst is an alcohol oxidase (AOX, E.C. 1.1.3.13) or alcohol oxidase homolog. AOX is a ubiquitous flavin-dependent enzyme that oxidizes lower primary alcohols to aldehydes using oxygen as an oxidizing agent. An example is depicted in Reaction Scheme 1.

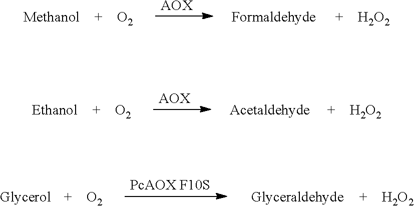

### Reaction Scheme 1

AOX may be sourced from methylotrophic yeast of the species Kloeckera, Torulopsis, Candida, Pichia, Hanseniaspora, and Metschnikowia. In an alternative aspect, the AOX is sourced from methanol-utilizing bacteria such as Methylococcus capsulatus, thermophilic soil fungi such as Thermoascus aurantiacus, and brown rot fungus such as Gloeophyllum trabeum. Alternatively, the AOX may be sourced from the white-rot basidiomycete Phanerochaete chrysosporium.

Methylotrophic yeasts are widely employed in fermentative processes for protein production and chemical synthesis. In many cases, these yeasts are used to generate proteins heterologously under control of the methanol-inducible AOX1 promoter. The endogenous AOX1 gene can be retained (Mut⁺strains), deleted (Muts), or deleted along with that of the minor alcohol oxidase AOX2 (Mut⁻). Generally, higher protein titers are achieved in strains capable of utilizing methanol as a carbon source, while AOX genes may be deleted to improve protein titers in non-methanol induced processes.

In an aspect, the AOX is sourced from Mut⁺ cells generated as a byproduct of methylotrophic yeast fermentation. Cell density in these processes can reach a final level of from about 350 g/L to about 450 g/L wet cells. When grown in methanol, AOX can comprise 30% of soluble cellular protein, 20% of cell-free extracts, and 80% of cell volume. Alternatively, AOX sequences used in this process may be sourced from organisms other than methylotrophic yeasts.

In an aspect, the oxidation biocatalyst is an inherently stable form of an AOX from thermophilic organisms such as Candida methanosorbosa (Tₒₚₜ=45° C.), Ogataea thermomethanolica (Tₒₚₜ=50° C.), or Phanerochaete chrysosporium (Tₒₚₜ=50° C.).

An AOX for use in the present disclosure may be utilized in the oxidation of ethylene glycol and in such instances is termed and ethylene glycol oxidase or EGOX.

### Galactose Oxidase

In an aspect, the oxidation biocatalyst is a member of the copper radical oxidase (CRO) family. For example, and without limitation, a copper radical oxidase suitable for use in the present disclosure is galactose oxidase (GAO, EC 1.1. 3.9). GAO is one of the most extensively studied alcohol oxidases with respect to both mechanistic investigations and practical applications. Other members in the copper radical oxidase family may be suitably employed in the present disclosure. GAO is a copper-dependent alcohol oxidase that oxidizes galactose residues either as monosaccharides or glycoconjugates that contain galactose at the nonreducing end. GAO is a novel metallo-radical complex comprising a protein radical coordinated to a copper ion in the active site. The unusually stable protein radical is formed from the redox-active side chain of a cross-linked tyrosine residue (Tyr-Cys).

### Glycerol Oxidase

In an aspect, the oxidation biocatalyst is a glycerol oxidase (GlyOx). GlyOx (E.C. 1.1.3. B4) catalyzes the oxidation of glycerol under the consumption of oxygen to form glyceraldehyde and hydrogen peroxide according to the following reaction:

CH₂OHCHOHCH₂OH+O_{2→}CHOCHOHCH₂OH+H₂O₂

The reaction proceeds in the absence of exogeneous cofactors. Natural glycerol oxidases containing copper-heme cofactors have been sourced from Botrytis allii, Aspergillus japonicus (AT 001 and AT 008), Aspergillus oryzae AT 105. Aspergillus parasiticus AT 462, Aspergillus flavus AT 853, Aspargillus tamarii AT 857, Aspergillus itaconicus AT 923, Aspergillus usamii AT 989, Neurospora crassa AT 003, Neurospora sitophila AT 045, Neurospora tetrasperma AT 053, and Penicilium sp. UT 1750.

### Sequences

In an aspect, biocatalysts suitable for use in the present disclosure may have any of SEQ ID No. 1 through SEQ ID No. 16.

### Supported Metal Catalyst

In an aspect, the metal catalyst comprises a transition-metal oxidation catalyst. In such aspects the metal oxidation catalyst is a supported transition-metal oxidation catalyst, alternatively a nanoparticle supported transition-metal oxidation catalyst. Hereinafter these are collectively designated, TMC. In an aspect, the support comprises carbon, silica, alumina, titania (TiO₂), zirconia (ZrO₂), a zeolite, or any combination thereof, which contains less than about 1.0 weight percent (wt. %), alternatively less than about 0.1 wt. % or alternatively less than about 0.01 wt. % SiO₂ binders based on the total weight of the support.

Suitable support materials are predominantly mesoporous or macroporous, and substantially free from micropores. For example, the support may comprise less than about 20% micropores. In an aspect, the support is a porous nanoparticle support. As used herein, the term “micropore” refers to pores with diameter <2 nm, as measured by nitrogen adsorption and mercury porosimetry methods and as defined by IUPAC. As used herein, the term “mesopore” refers to pores with diameter from ca. 2 nm to ca. 50 nm, as measured by nitrogen adsorption and mercury porosimetry methods and as defined by IUPAC. As used herein, the term “macropore” refers to pores with diameters larger than 50 nm, as measured by nitrogen adsorption and mercury porosimetry methods and as defined by IUPAC.

In an aspect, the support comprises a mesoporous carbon extrudate having a mean pore diameter ranging from about 10 nm to about 100 nm and a surface area greater than about 20 m² g⁻¹ but less than about 300 m² g⁻¹. Supports suitable for use in the present disclosure may have any suitable shape. For example, the support may be shaped into 0.8-3 mm trilobes, quadralobes, or pellet extrudates. Such shaped supports enable the used of fixed trickle bed reactors to perform the final oxidation step under continuous flow.

In one or more aspects, the metal comprises a Group 8 metal (e.g., Re, Os, Ir, Pt, Ru, Rh, Pd, Ag), a 3d transition metal, an early transition metal, or combinations thereof. In an aspect, the TMC comprises gold, Au.

In an aspect, the TMCs comprise platinum and gold and are heterogeneous, solid-phase TMCs. In such aspects, suitable catalyst supports include, without limitation, carbon, surface treated aluminas (such as passivated aluminas or coated aluminas), silicas, titanias, zirconias, zeolites, montmorillonites, and modifications, mixtures or combinations thereof. The catalyst support may be treated to promote the preferential deposition of platinum and gold on the outer surface of the support so as to create a shell type TMC. The platinum and gold-containing compounds that function as a TMC may be produced by any suitable methodology. For example, the platinum and gold-containing TMCs may be produced using deposition procedures such as incipient wetness, ion-exchange and deposition-precipitation.

In other aspects, TMC comprises metal phases that are monometallic or multimetallic combinations of Cu, Ag, Au, Ni, Pd, Pt, and Ir. The activity, selectivity, and stability of the active phases can be modulated with dopants of early 3d, 4d, and 5d transition metals, or heavy post transition metals such as Sn, Sb, and Bi. In some aspects, metals (e.g., Group 1 metals) are intercalated into the metal lattice to modulate catalyst properties. In an aspect, salt precursors of the active phases are deposited onto a support of the type disclosed herein using any suitable methodology. For example, deposition of the active phases may be carried out using techniques such as incipient wetness impregnation, bulk adsorption impregnation, or deposition precipitation.

In an aspect, the deposited salt precursor of the active phase is then converted to the active phase via Liquid Phase Reduction (LPR) with a suitable salt (e.g., formate salt) at temperatures of less than about 100° C. or via Gas Phase Reduction (GPR) at temperatures ranging from about 200° C. to about 500° C. or alternatively from about 200° C. to about 450° C. In an aspect, the finishing catalyst comprises gold and calcination in air at temperatures of equal to or greater than about 150° C.

In an aspect, the amount of active phase loaded onto a support of the type disclosed herein is less than about 2.0 weight percent (wt. %), alternatively less than about 1.5 wt. % or alternatively less than about 1.0 wt. % based on the total weight of the TMC finishing catalyst. In an aspect, the amount of active phase loaded onto a support of the type disclosed herein is equal to or less than about 0.5 wt. % based on the total weight of the TMC finishing catalyst. In an aspect, the radial distribution of the active phase across the support is anisotropic where the active phase is substantially concentrated in a <500 μm annulus near the surface of the extrudate support in a “core-shell” configuration. A TMC finishing catalyst of the type disclosed herein may be characterized by a productivity for the conversion of aldehyde functionalities to carboxylic acids of equal to or greater than about 0.05 mol acid g⁻1 active metal h⁻1 or equal to or greater than about 0.1 mol acid g⁻1 active metal h⁻1 at selectivities from about 70% to about 90%, alternatively equal to or greater than about 70%, alternatively equal to or greater than about 80%, alternatively equal to or greater than about 85%, or alternatively equal to or greater than about 90%. In such aspects, the TMC finishing catalyst exhibits conversions of from about 60% to about 95%, alternatively equal to or greater than about 70%, alternatively equal to or greater than about 80%, or alternatively equal to or greater than about 90%. Such TMC finishing catalysts may display a steady state leaching amount of from about 1 ppb to about 100 ppb, alternatively less than about 100 ppb or alternatively less than about 90 ppb. In an aspect, a TMC finishing catalyst of the type disclosed herein may be utilized in a temperature range of from about 40° C. to about 120° C. alternatively form about 40° C. to about 110° C. or alternatively from about 50° C. to about 100° C. at pressures ranging from about 10 bar to about 100 bar, alternatively from about 20 bar to about 100 bar or alternatively from about 20 bar to about 90 bar.

In some aspects, the finishing catalyst is an isomerization catalyst. Any isomerization catalysts compatible with the other components of the HVCOS may be utilized. In some aspects, the isomerization catalyst comprises a zeolite.

### Chemical Catalysts

In an aspect, the finishing catalyst is a small molecule chemical catalyst such as an acid or base. Examples of acids or bases suitable for use as a finishing catalyst include without limitation hydrochloric acid, sulfuric acid, formic acid, sodium hydroxide and urea.

### Cofactors

In an aspect, the biocatalysts suitable for use in an HVCOS of the type disclosed herein may further include one or more purified cofactors. Herein a cofactor refers to non-protein chemical compound that modulates the biological activity of the biocatalyst. Many enzymes require cofactors to function properly. Nonlimiting examples of purified enzyme cofactors suitable for use in the present disclosure include thiamine pyrophosphate, NAD+, NADP+, pyridoxal phosphate, methyl cobalamin, cobalamine, biotin, Coenzyme A, tetrahydrofolic acid, menaquinone, ascorbic acid, flavin mononucleotide, flavin adenine dinucleotide, and Coenzyme F420. Such cofactors may be included in the biocatalyst preparation and/or be added at various points during the reaction. In some aspects, cofactors included with the biocatalyst preparation may be readily regenerated with oxygen and/or may remain stable throughout the lifetime of the enzyme(s).

### Catalase

As will be understood by one of ordinary skill in the art with the benefit of the present disclosure, reactions of the type disclosed herein (e.g., biocatalyst oxidation of ethylene glycol) may result in the production of byproducts (e.g., hydrogen peroxide) that can detrimentally impact other components of the reaction mixture. For example, hydrogen peroxide may degrade the biocatalyst resulting in a loss of catalytic activity. In such aspects, mitigation of the detrimental effects of hydrogen peroxide may be carried out such as by the introduction of a catalase (E.C. 1.11.1.61), the use of a hydrogen peroxide-resistant biocatalyst or combinations thereof.

### Biocatalyst Forms and Expression Systems

In an aspect, a biocatalyst of the type disclosed herein is a wild type enzyme, a functional fragment thereof or a functional variant thereof. “Fragment” as used herein is meant to include any amino acid sequence shorter than the full-length biocatalyst (e.g., AOX), but where the fragment maintains a catalytic activity sufficient to meet some user or process goal. Fragments may include a single contiguous sequence identical to a portion of the biocatalyst sequence. Alternatively, the fragment may have or include several different shorter segments where each segment is identical in amino acid sequence to a different portion of the amino acid sequence of the biocatalyst but linked via amino acids differing in sequence from the biocatalyst. Herein, a “functional variant” of the biocatalyst refers to a polypeptide which has at one or more positions of an amino acid insertion, deletion, or substitution, either conservative or non-conservative, and wherein each of these types of changes may occur alone, or in combination with one or more of the others, one or more times in a given sequence but retains catalytic activity.

In the alternative or in combination with the aforementioned mutations, the biocatalyst may be mutated to improve the catalytic activity. Mutations may be carried out to enhance the protein or a homolog activity, increase the protein stability in the presence of substrates and products such as formaldehyde, acetaldehyde, glycolaldehyde and/or hydrogen peroxide, and increase protein yield.

Herein, reference has been made to “sources” of biocatalysts. It is to be understood this refers to the biomolecule as expressed by the named organism. It is contemplated the biocatalyst may be obtained from the organism or a version of said biocatalyst (wildtype or recombinant) provided as a suitable construct to an appropriate expression system.

In an aspect, any biocatalyst of the type disclosed herein may be cloned into an appropriate expression vector and used to transform cells of an expression system such as E. coli, Saccharomyces sp., Pichia sp., Aspergillus sp., Trichoderma sp., or Myceliophthora sp. A “vector” is a replicon, such as plasmid, phage, viral construct or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the gene of a biocatalyst disclosed herein is provided as a recombinant sequence in a vector where the sequence is operatively linked to one or more control or regulatory sequences. “Operatively linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and are used herein refer to polynucleotide sequences, which are necessary to affect the expression of coding sequences to which they are operatively linked. Expression control sequences are sequences that control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”. “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

### Advantages

Disclosed herein are molecular manufacturing platforms for the chemoenzymatic production of VACs from renewable feedstocks such as sugars and alcohols. In an aspect, the presently disclosed VACs are produced at commercial scale and at purities of equal to or greater than about 70%, alternatively equal to or greater than about 80%, alternatively equal to or greater than about 90%.

### EXAMPLES

The subject matter having been generally described, the following examples are given as particular aspects of the disclosure and are included to demonstrate the practice and advantages thereof, as well as aspects and features of the presently disclosed subject matter. It should be appreciated by those of skill in the art that the techniques disclosed in the examples which follow represent techniques discovered by the inventors to function well in the practice of the present subject matter, and thus can be considered to constitute preferred modes for its practice. However, those of skill in the art should, in light of the present disclosure, appreciate that many changes can be made in the specific aspects which are disclosed and still obtain a like or similar result without departing from the scope of the instant disclosure. It is understood that the examples are given by way of illustration and are not intended to limit the specification or the claims to follow in any manner.

### Mutant Enzyme

Sugars are not known as native substrates of UPOs, and thus, UPOs will be engineered to improve specific activity. Kₘ, and k_{cat} on glucose or oxidized glucose derivatives. This will be accomplished through screening a panel of UPOs for baseline activity and other suitable properties such as optimal pH, thermo- and chemical stability, and expression level in both engineering (Escherichia coli) and production (fungal) hosts. Once a suitable enzyme is selected, it will be subjected to directed evolution and rational design methods to improve enzymatic properties.

Rather than rely on fermentation and/or petrochemistry, a new process is disclosed herein that relies on enzyme reactions coupled with catalysts to provide for generation of products at lower temperatures and with fewer biproducts. The use of enzymes can result in the reactions to generate intermediates and products without the need for active fermentation. When coupled with catalytic chemistry, the two processes can generate products from reactants at high selectivities and yields while limiting the generation of CO2.

### Example 1

Production of Gluconic Acid from Glucose Using a Glucose Oxidase Catalyst

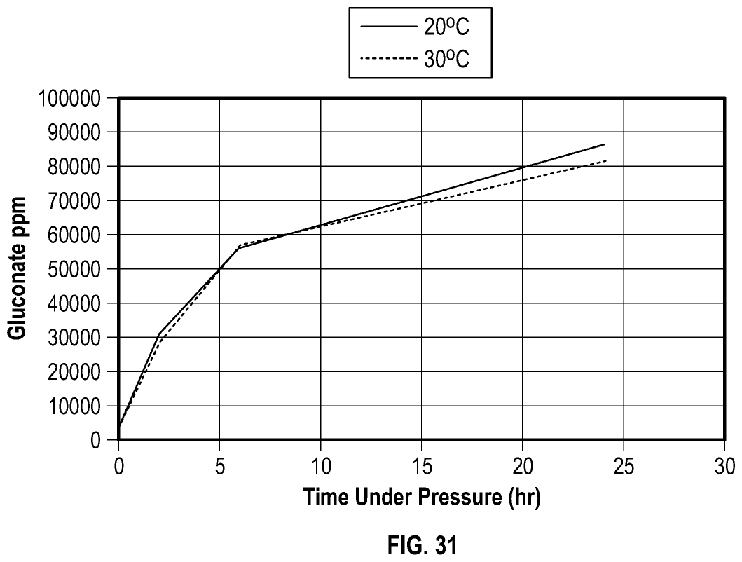

A 50 mL reaction was conducted in a 200 mL vessel pressurized to 100 psi with O₂. The vessel was charged with 20% dextrose, 100 mM citrate buffer, pH 5, 0.0002% glucose oxidase, and 0.0002% catalase. The reaction was stirred at 500 rpm at 20° C. or 30° C. At approximately 25 hours, the reaction was stopped and determined by reverse phase HPLC-MS to produce 45,000 g of gluconic acid per gram of glucose oxidase. The results of HPLC analysis are shown in FIG. 31.

### Example 2

Generation of Glucaric Acid from Glucose and Gluconate

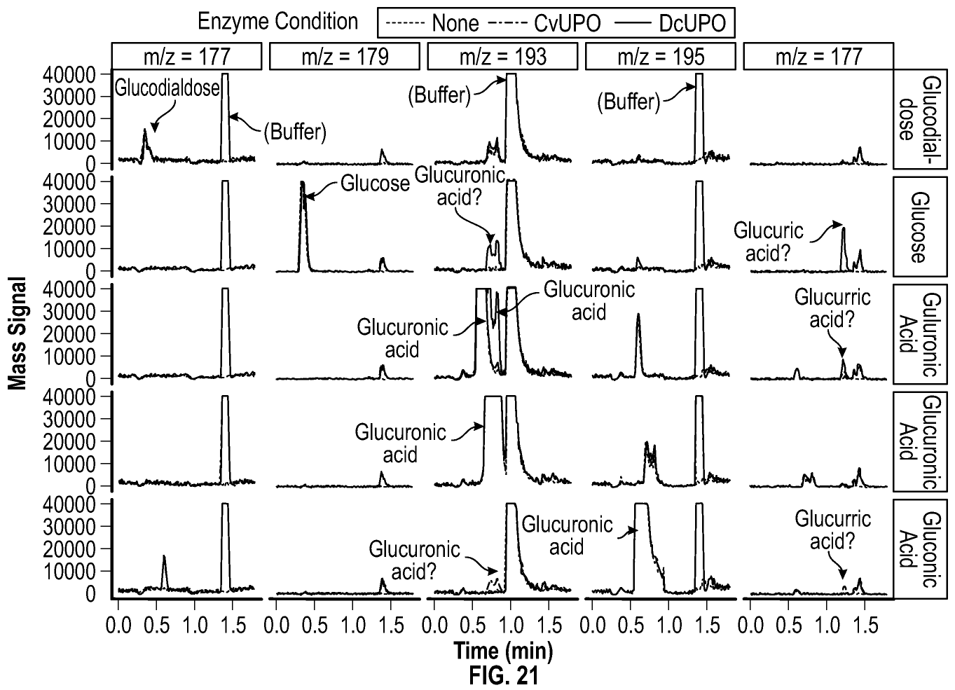

Collariella viriscens (CviUPO) and Daldinia caladariorum (DcaUPO) UPOs were expressed in E. coli, purified via affinity chromatography, and tested for baseline activity on glucose, gluconic acid, glucodialdose, guluronic acid, and glucuronic acid. The reactions were set up in 96-well plate format containing 0.1 g/L enzyme, either sodium citrate (pH 4) or potassium phosphate (pH 6) buffer, 2 mM H₂O₂, and glucose or glucose oxidation products at 2 mM. Reactions were allowed to run for 20 hours and then analyzed via HPLC-MS. Referring to FIG. 21, the baseline production of glucaric acid from glucose and/or glucose oxidation products using as catalysts Collariella viriscens (CviUPO) or Daldinia caladariorum (DcaUPO) as measured by HPLC-MS is shown. All product traces are shown post-reaction. Notably, DcaUPO generated glucarate from glucose while CviUPO was found to generate small quantities of glucaric acid from gluconate. Glucarate was only generated at pH 4 for both enzymes. At pH 6,2-ketogluconate was generated by both enzymes with no glucarate production present.

### Example 3

### Generation of L-Guluronic Acid in Parr Bomb

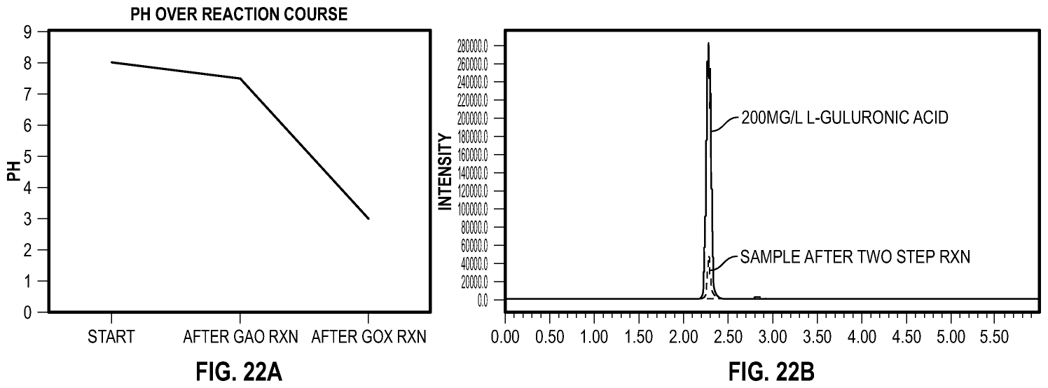

L-guluronic acid production was demonstrated on a benchtop scale using GAO-mut1 and GOX enzymes added to a Parr bomb vessel pressurized to 100 psi with oxygen. In an initial experiment, a solution of 50 mM sodium phosphate buffer, pH 8, containing glucose 10% weight/volume (w/v %), GAO-mut1 0.02 w/v %, and catalase 0.001 w/v % was prepared (50 mL) and added to the inner chamber of the vessel. The stirred reaction proceeded at 20° C. for 20 hours. The GAO Mut-1 enzyme was then removed by filtering through a 30 kD MWCO centrifugal device. In the second reaction phase, GOX and catalase were both added to a concentration of 0.001 w/v %. The reaction was again allowed to proceed with stirring at 20° C. for 20 hours. It was noted that during the reaction, the pH declined from 8 to 3, presumably from the generation of the acid species gluconic and L-guluronic acid after the addition of GOX as shown in FIG. 22A. HPLC-MS analysis of the final reaction mix demonstrated production of around 0.2-0.3% L-guluronic acid (2-3% molar yield), 2% gluconate, and an unspecified quantity of glucodialdose as shown in FIG. 22B.

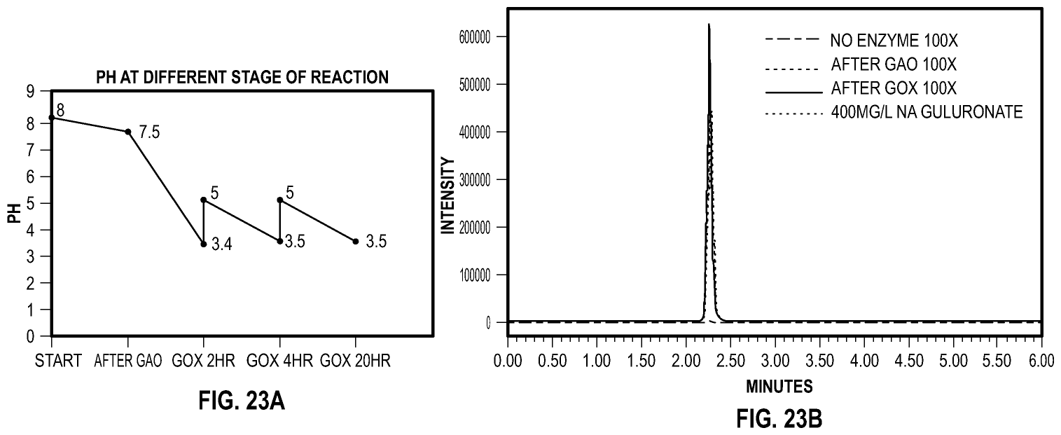

To improve yield, a second experiment was performed in which the pH was controlled during the acid-generating second step. First, a solution of 50 mM sodium phosphate buffer, pH 8, glucose 15 w/v %, GAO-mut1 0.02 w/v %, and catalase 0.001 w/v % was added to the Parr bomb (50 mL) and stirred at a temperature of 20° C. for 20 hours to generate glucodialdose. In the second step, GOX 0.001 w/v % and an additional catalase 0.001 w/v % was added and allowed to proceed at the same conditions for an additional 20 hours. The reaction was stopped, the pH adjusted to 5 at 2 and 4 hours, then repressurized and allowed to react until 20 hours elapsed as shown in FIG. 23A. Upon LC-MS analysis, a higher concentration (approximately 5% or 31% molar yield) of L-guluronate was generated compared to the previous run. The results are shown in FIG. 23B.

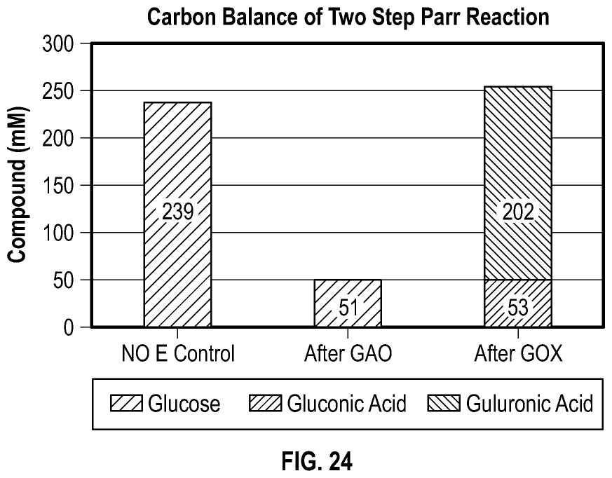

A third run was performed to further increase yield by adding a higher concentration of GAO-mut1 and adjusting pH to 6 in the second catalysis step. First, a solution of 50 mM sodium phosphate buffer, pH 8, approximately glucose 4 w/v %, GAO-mut1 0.1 w/v %, and catalase 0.001 w/v % was added to the Parr bomb at a volume of 50 mL and stirred at a temperature of 20° C. for 20 hours to generate glucodialdose. In the second step, GOX 0.001 w/v % and an additional catalase 0.001 w/v % was added and allowed to proceed at the same conditions for another 20 hours. The reaction was periodically paused and the pH adjusted to 6. Following the reaction with GAO-Mut1, the concentration of glucose dropped from the initial loading of 4.3 w/v % to 0.9 w/v %. After addition of GOX and reaction for 20 hours, a mixture of 1.0 w/v % gluconic and 3.6 w/v % L-guluronic acid (85% molar yield) was produced. The results are presented in FIG. 24.

### Example 4

Generation of GAO Mutant for Producing Glucodialdose from Glucose

In support of Pathway A, a GAO mutant was engineered capable of converting glucose to glucodialdose. Following directed evolution and rational enzyme engineering, the improved GAO mutant exhibits a specific activity of >35 U mg−1 on glucose. Directed Evolution

Directed evolution of thirty sites within 10 Å of the catalytic copper was performed on a parent sequence containing the following added mutations: 1) R330K, Q406T, W290F discovered by to introduce less than 1 U mg⁻¹ activity on glucose to GAO, 2). C₃₈₃S was found to lower the KM of the enzyme on galactose, and 3) Y405F and Q406E was found to enhance activity on a D-N-acetyl glucosamine substrate. Other mutations described in Table I were found to have neutral or deleterious effects on glucodialdose-generating activity. The new combination sequence was designated GAO-Mut1. The full sequence of the expressed construct is given in SEQ ID No. 15.

TABLE 1

| | | Addition |
|---|---|---|
| Name | Staring Mut | mutations |
| M1 | wild-type | S10P M70V |
| | | P136P G195E |
| | | V494A N535D |
| M-RQW | M1 | R330K Q406T |
| | | W290F I463P |
| GAO | M-RQW | C383S |
| GAO-mut1 | GAO | Y405F Q406E |
| GAO-mut2 | GAO | F194T |
| GAO-mut3 | GAO | C383E |
| GAO-mut4 | GAO | N245R |
| GAO-mut5 | GAO | Q326E Y329K |

Selected positions in GAO-Mut1 were mutated via the Quikchange method to all 20 amino acids using primers containing NNS codons. The constructs were then screened in the following manner: Colonies were picked and used to inoculate one well each in a 96-well deepwell plate prefilled with Luria-Bertani (LB) broth. The grown clones were then used to inoculate autoinduction media in a separate 96-well deepwell plate for protein expression. Harvested cells were lysed with Bacterial Protein Extraction Reagent (B-PER) and the lysate screened for oxidase activity using a colorimetric 2,2′-azino-bis (3-ethylbenzothiazoline-6-sulfonic acid (ABTS) assay which detects hydrogen peroxide.

In short, lysate was assayed for activity with and without exposure to heat. To assay activity in the absence of a heat challenge, lysate was diluted 50 times. A volume of 5 μL of the diluted lysate was combined with ABTS assay solution (final concentration of 2 w/v % glucose, 0.0125 mg/ml horseradish peroxidase (HRP), 50 mM sodium phosphate buffer at pH 8, and 0.05% ABTS) to a final volume of 200 μL and the change in absorbance at 405nm was monitored until the reaction was complete. To assay residual activity after a heat challenge, 50 μL lysate was incubated for ten minutes at 50° C. and 20 μL of the heat-treated lysate was added to the ABTS solution before monitoring change in absorbance at 405 nm. Specific activity was calculated from the formulas below using the linear portion of the curve to measure ΔA405/min and taking the extinction coefficient of ABTS at 405 nm as 36.8 mM⁻¹. cm⁻¹. Division by 2 is done to account for the fact that one molecule of H₂O₂oxidizes two molecules of ABTS by HRP.

$$Units {mg}^{- 1} = \frac{Δ {A}_{405} {min}^{- 1}}{36.8 / 2 \times ( pathlength in cm ) \times ( mg enzyme ) / ( ml reaction mixture )}$$

$$Units {ml}^{- 1} = \frac{Δ {A}_{405} {min}^{- 1}}{36.8 / 2 \times ( pathlength in cm ) \times ( ml enzyme ) / ( ml reaction mixture )}$$

Mutant lysates exhibiting a AA405/min greater than the GAO-Mut1 were chosen for further characterization. Following identification of the mutation by DNA sequencing, hits were expressed, purified, and assayed for specific activity and thermostability as assessed by the temperature at which one half maximal activity was observed (T₅₀). Mutants were purified from 5 mL culture with auto-induction medium in 24-well plates. Harvested cells were lysed with Bacterial Protein Extraction Reagent (B-PER) and the lysate was spun down at 15,000 rcf for 30 min at 4° C. His-tagged protein in the lysate supernatant was purified by immobilized metal affinity chromatography with HisPur™ Ni-NTA Spin Plates. The eluted protein sample was diluted with 100 mM potassium phosphate buffer pH=7.5 with 0.5 mM CuSO₄, and the specific activity was measured using the ABTS assay outlined above.

The T₅₀ was measured by heating the protein in the absence of substrate, cooling, and then measuring residual activity using the ABTS assay. Heating was accomplished by diluting the protein to a concentration of 2.5 mg/L in a volume of 100 mM phosphate buffer at pH 7.5, aliquoting 50 μL into a row of a 96-well PCR plate, and incubating over a temperature gradient sufficient to capture maximal and minimal enzyme performance for ten minutes. Promptly after heating, the mixture was cooled on ice and the ΔA405/min of 20 μL of enzyme solution in 200 μL of final volume of ABTS solution was measured as described above.

Promising point mutants that could beneficially be combined in the Mut1 background included A193R, D404H, F441Y, and A172V (Table 3). These mutations were combined

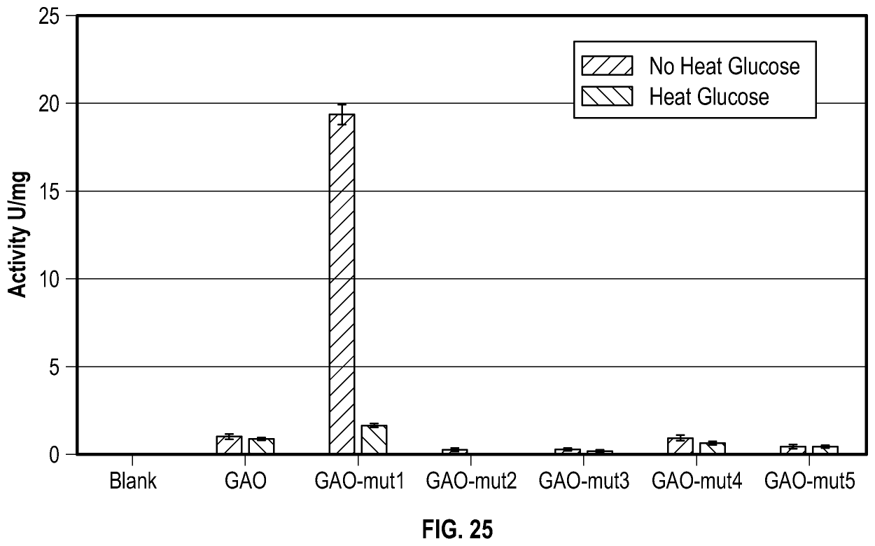

into a single combination mutant named GAO-Mut47 which exhibited a specific activity of 27.3 U mg⁻¹ and a T₅₀ of 56.8° C. The results are presented in FIG. 25 and Table 2.

TABLE 2

| Name | Mutations | U/mg | T₅₀ ° C. | K_{c}at s⁻¹ | Kₘ mM |
|---|---|---|---|---|---|
| M-RQW-S | | 1.1 | 56.8 | 31.4 | 2168.3 |
| GAO-mut1 | Y405F Q406E | 14.0 | 51.8 | 30.2 | 93.1 |
| GAO-mut6 | Y405F Q406E S383C | 6.1 | 41.5 | 36.6 | 412.0 |
| GAO-mut7 | Y405F Q406E F441Y | 16.9 | 53.6 | 27.3 | 42.7 |
| GAO-mut8 | Y405F Q406E D404H | 15.0 | 53.7 | 30.7 | 83.9 |
| GAO-mut9 | Y405F Q406E G461A | 13.4 | 53.2 | 27.8 | 83.7 |
| GAO-mut10 | Y405F Q406E I462R | 12.1 | 53.2 | 31.6 | 130.7 |
| GAO-mut11 | Y405F Q406E A172V | 21.2 | 48.5 | 39.5 | 72.6 |
| GAO-mut12 | Y405F Q406E A193R | 15.4 | 56.3 | 28.0 | 64.8 |
| GAO-mut13 | Y405F Q406E A193T | 14.6 | 53.8 | 30.4 | 75.5 |
| GAO-mut14 | Y405F Q406E D404H F441Y | 18.8 | 55.0 | 26.5 | 29.7 |
| GAO-mut15 | Y405F Q406E G461A I462R | 12.2 | 54.0 | 24.8 | 79.1 |
| GAO-mut17 | Y405F Q406E D404H F441Y G461A I462R | 18.2 | 55.3 | 23.6 | 25.5 |
| GAO-mut18 | Y405F Q406E A193T D404H F441Y G461A I462R | 18.1 | 56.6 | 24.1 | 28.0 |
| GAO-mut19 | Y405F Q406E A193T D404H F441Y G461A I462R S383C | 13.3 | 46.3 | 24.5 | 70.8 |
| GAO-mut20 | Y405F Q406E A193T D404H F441Y G461A I462R S383C A172V | 21.4 | 37.9 | 35.6 | 58.2 |
| GAO-mut21 | Y405F Q406E F441Y G461A I462R | 18.3 | 53.8 | 24.2 | 29.6 |
| GAO-mut22 | Y405F Q406E A193T D404H F441Y G461A I462R A172V | 23.6 | 51.5 | 29.5 | 26.4 |
| GAO-mut23 | Y405F Q406E A193R D404H F441Y G461A I462R A172V | 21.1 | 57.5 | 27.2 | 26.8 |
| GAO-mut47 | Y405F Q406E A193R D404H F441Y A172V | 27.3 | 56.8 | 35.0 | 25.2 |
| GAO-mut58 | Y405F Q406E D404H F441Y A172V | 27.1 | 52.9 | 35.4 | 26.6 |

### Rational Engineering

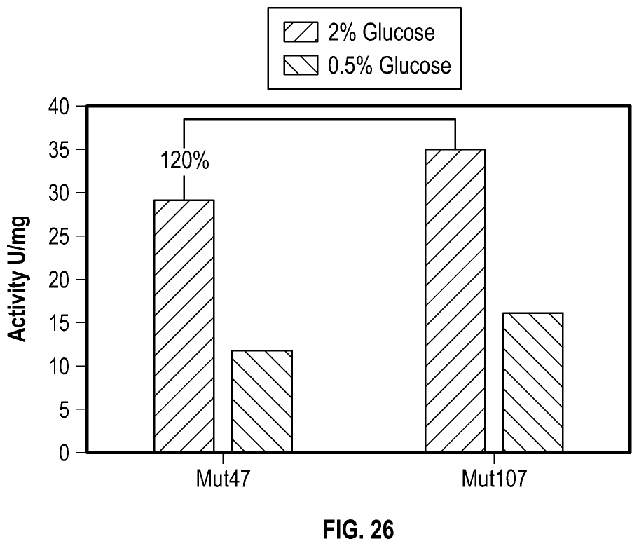

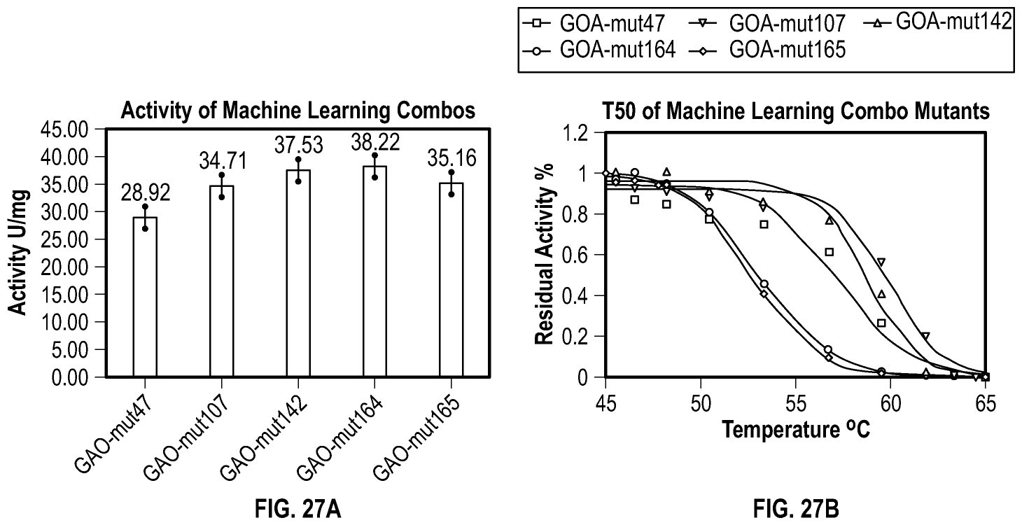

Rational engineering of GAO to further accept a glucose substrate and identify stabilizing mutations was accomplished with a combination of computational methods based on structural and multiple sequence alignment (MSA) data. Previously, the GAO-M-RQW-S (the GAO-Mut1 sequence without the Y405F and Q406E mutations) were found to could accept both glucose and gluconate as substrate. The GAO-M-RQW-S sequence with and without N-terminal His tags are presented in SEQ ID NO. 16 and SEQ ID NO. 17, respectively. As efforts were underway to produce a GAO active on both substrates, rational design was performed on the GAO-M-RQW-S sequence rather than GAO-Mut1. Structural methods employed included applying FoldX (40 predicted mutations) and PROSS (80 mutations) to a modified form of the PDB structure 2WQ8 to contain the GAO-M-RQW-S mutations. MSA-based predictions were prepared (34 mutations) and (28mutations) and applied to a 185-member MSA. This MSA was generated from an initial set of 1000 sequences curated with JALVIEW to remove sequences with 98% redundancy and retain only sequences experimentally verified as carbohydrate oxidases. In total, 202-point mutants were screened using the same methods described above for screening the directed evolution clones. Thirty-nine hits were identified from an initial screen and sixteen were reidentified from a second round of screening. Upon generation of combo mutants in the best combination mutant from the directed evolution step (GAO-Mut47), the mutations N66S, S306A, S311F, and Q486L were identified as complementary and beneficial while N281, Y189W, S331R, A378D, and R459Q were deemed detrimental in this background (Table 3). The final GAO-Mut107 construct containing the Mut47 mutations and N66S. S306A, S311F, and Q486L exhibits a specific activity of 34.96 U mg⁻1 on 2% glucose and a T₅₀ of 60.56° C. The results are presented in FIG. 26. Additional mutations identified from machine learning algorithms were later incorporated to generate GAO-mut142 and GAO-mut164. The results of activity assays of these additional mutants along with the GAO-mut47 and GAO-mut107 enzymes are presented in FIG. 27.

TABLE 3

| | | | | Fold | |
|---|---|---|---|---|---|
| Clone | Mutations from Mut47 | New Mutations | U/mg | Improvement | T50° C. |
| Mut47 | | | 31.11 | 1.00 | 57.64 |
| GAO-mut68 | N28I | N28I | 30.84 | 0.99 | 56.76 |
| GAO-mut69 | N28I N66S | N66S | 33.68 | 1.08 | 59.00 |
| GAO-mut70 | N28I N66S Y189W | Y189W | 31.80 | 1.02 | 59.91 |
| GAO-mut71 | N28I N66S Y189W S306A | S306A | 32.66 | 1.05 | 59.48 |
| GAO-mut72 | N28I N66S Y189W S306A S311F | S311F | 33.87 | 1.09 | 60.81 |
| GAO-mut73 | N28I N66S Y189W S306A S311F | S331R | 27.56 | 0.89 | 59.87 |
| | S331R | | | | |
| GAO-mut74 | N28I N66S Y189W S306A S311F | A378D | 25.57 | 0.82 | 58.94 |
| | S331R A378D | | | | |
| GAO-mut75 | N28I N66S Y189W S306A S311F | R459Q | 23.51 | 0.76 | 58.49 |
| | S331R A378D R459Q | | | | |
| GAO-mut76 | N28I N66S Y189W S306A S311F | V477D | 19.22 | 0.62 | 59.17 |
| | S331R A378D R459Q V477D | | | | |
| GAO-mut77 | N28I N66S Y189W S306A S311F | Q486L | 24.57 | 0.79 | 59.88 |
| | S331R A378D R459Q V477D Q486L | | | | |
| GAO-mut107a | N66S S306A S311F Q486L | Removed N28I, | 34.96 | 1.20 | 60.56 |
| | | Y189W, S331R, | | | |
| | | A378D, R459Q, | | | |
| | | and V477D | | | |
| GAO-mut142b | N66S S306A S311F Q486L H40C | H40C | 37.53 | 1.29 | 58.76 |
| GAO-mut164b | N66S S306A S311F Q486L H40C L71C | L71C | 38.22 | 1.32 | 52.97 |

Bolded mutations are beneficial in a Mut47 background A193R D404H F441Y A172V
Data collected in a separate experiment from other data. Fold improvement is calculated compared to an internal Mut47 control.
Data collected in a separate experiment from other data. Fold improvement is calculated compared to an internal Mut47 control.

One-Step Parr Bomb Reaction with GAO-Mut47 to Produce D-Glucodialdose

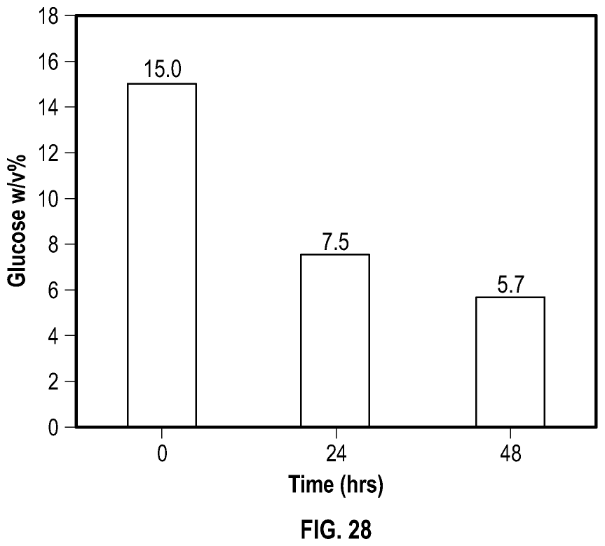

A 50 mL reaction was conducted in a 200 mL vessel pressurized to 100 psi. The vessel was charged with 50 mM sodium phosphate pH 8 buffer, 50 μM CuSO₄, 15 w/v % glucose, 0.005 w/v % catalase, 0.001% horseradish peroxidase, and 0.001 w/v % of an engineered GAO. The reaction was stirred at 500 rpm, 11° C. for 48 hours. Samples were taken at 0, 24, and 48 hours then assayed with HPLC to measure residual glucose. The results are shown in FIG. 28.

Two-Step Parr Bomb Reaction with GAO-Mut47 to Produce L-Guluronic Acid

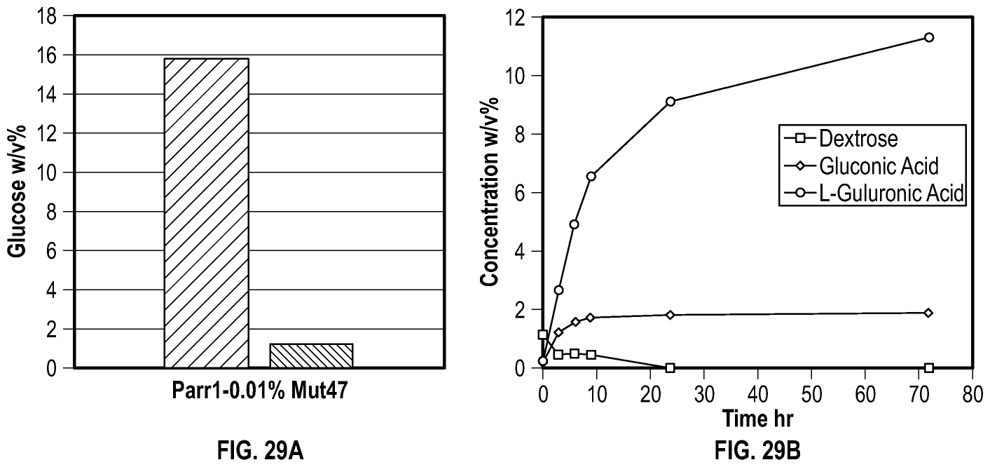

A 50 mL reaction was conducted in a 200 mL vessel pressurized to 100 psi. The vessel was charged with 50 mM sodium phosphate pH 8 buffer, 50 μM CuSO₄, 15 w/v % glucose, 0.005 w/v % catalase, 0.001% horseradish peroxidase, and 0.01 w/v % engineered GAO. The reaction was stirred at 500 rpm, 11° C. for 72 hours to generate glucodialdose from glucose. In the second step, 0.002 w/v % GOX and an additional 0.001 w/v % catalase was added and allowed to proceed at the same conditions for another 24 hours. The reaction was periodically paused and the pH adjusted to 7. The results are presented in FIG. 29A. The glucose concetration at time, zero (0) is shown as the bar on the left and the glucose concentration, following the first enzymatic step, particularly, reaction with the GAO enzyme composition, is shown on the right.

Following the reaction with GAO-Mut47, the concentration of glucose dropped from the initial loading of 16% w/v to 1.5% w/v. After addition of GOX and reaction for 24 hours, a mixture of 2.0% w/v gluconic and 12% w/v L-guluronic acid (75% molar yield) was produced. The results are graphed in FIG. 29B.

### Example 5

Generation of GAO Mutant for Producing L-Guluronic Acid from Gluconate or Gluconolactone

Combination mutants generated during the rational engineering of a GAO mutant active on glucose were screened for activity on gluconate. We surmised that there may be mutants active on gluconate among the combinations generated based on a GAO-M-RQW-S background because the parent construct already demonstrated about 1 U mg−1 specific activity on gluconate.

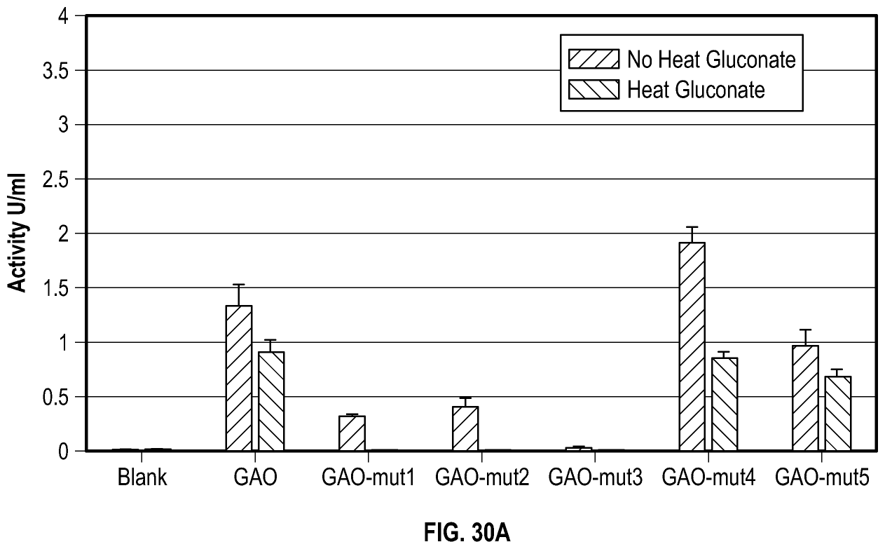

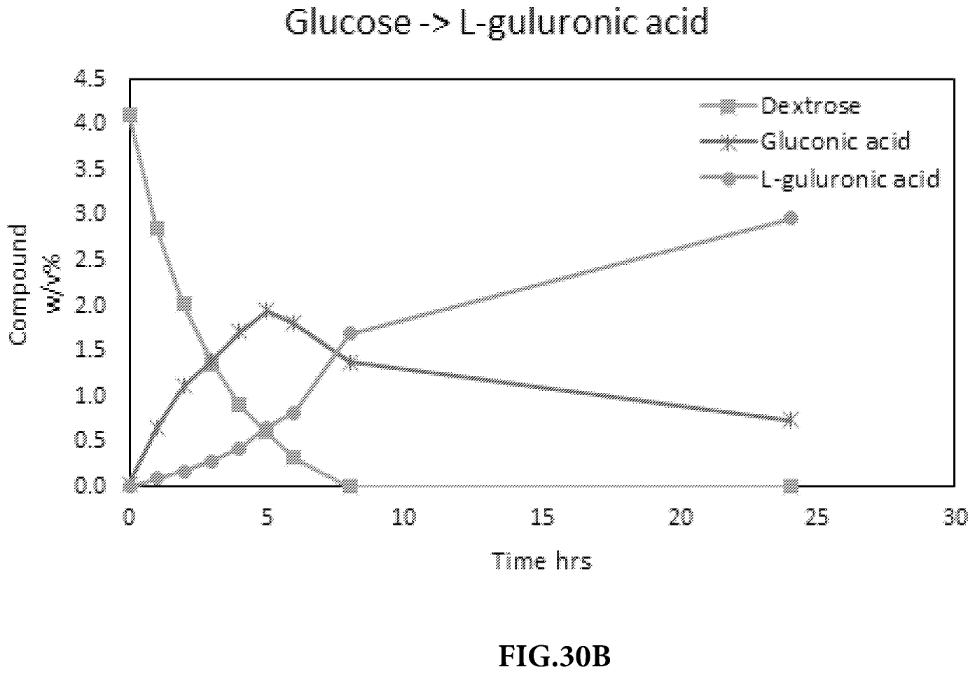

Screening on purified protein with 2% gluconate revealed Mut 49 (N66W, A172V, and Y189W) and Mut62 (N66S, A172V, Y189W, S306A, S311F, S331R, A178D, Q486L) as highly active on gluconate with specific activities of about 4 and 6 U mg⁻¹. The results are presented in FIG. 30A.

One-Step Parr Bomb Reaction with GAO-Mut62 to Produce L-Guluronic Acid

A 50 ml reaction was conducted in a 200 mL vessel pressurized to 100 psi with O₂. The vessel was charged with 50 mM sodium phosphate pH 8 buffer, 50 μM CuSO₄, 4 w/v % glucose, 0.005 w/v % catalase, 0.001 w/v % horseradish peroxidase, 0.0002 w/v % GOX, and 0.05 w/v % engineered GAO-mut62. The reaction was stirred at 500 rpm, 11° C. for 24hr. The reaction was periodically paused and the pH adjusted to 7.5. After 24 hours of reaction, 3 w/v % of L-guluronic acid and 1 w/v % of gluconic acid was generated from 4% glucose. The results are presented in FIG. 30B.

### Example 6

Production of 2-Ketoglucose from Glucose Using A Pyranose Oxidase Catalyst

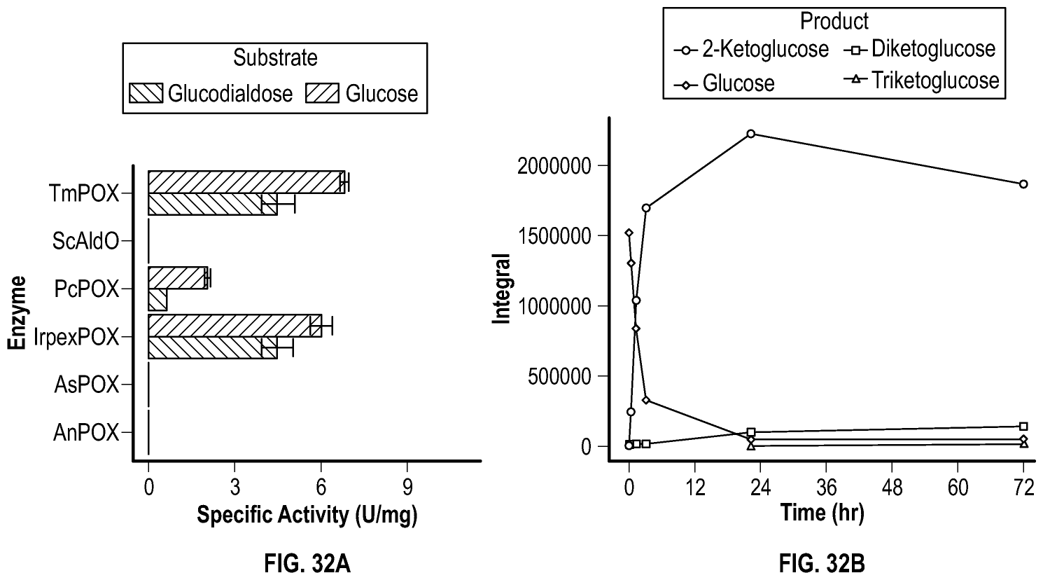

A well characterized enzyme, POX exhibits native specific activities of 10 U mg−¹using a tightly bound or covalent FAD cofactor that can be purified along with the protein from the expression host. POX is a flavin-dependent enzyme found in lignocellulose-degrading fungi that oxidizes glucose to 2-ketoglucose concomitantly with hydrogen peroxide formation. Recombinant Trametes hirsuita POX is currently used in the food industry for baking, providing some evidence that the enzyme can be produced at low cost. Table 4 presents previously characterized POX enzymes of interest for use in this process. A unit is defined as 1 umol of substrate consumed min⁻¹. Based on an in-house empirical evaluation, the Irpex lacteus, Trametes multicolor, and Phanerochaete chrysosporium POXs have sufficient starting activity. This is presented graphically in FIG. 32 and Table 4.

TABLE 4

| | Specific Activity | Beneficial | Crystal |
|---|---|---|---|
| Organism | (U mg⁻¹) | Mutations | Structure |
| Irpex lacteus | 6.0 (in-house | | No |
| | evaluation), 86 | | |
| Kitasatospora | 15.9 | | No |
| aureofasciens | | | |
| Lyophyllum shimeji | 1.92 | | No |
| Peniophora sp. P20xA1 | NA | E542K | Yes |
| | | T158A | |
| Phanerochaete | 2.0 (in-house | | Yes |
| chrysosporium | evaluation), 31.4 | | |
| Phlebiopsis gigantea | 22.5 (P₂OxB₂H | K312E | No |
| | K312E) | | |
| Polyporus obtusus | 8.5 | | No |
| Trametes multicolor | 6.8 (in-house | E542K | Yes |
| | evaluation), | | |
| Tricholoma matsutake | 25.8 | | No |

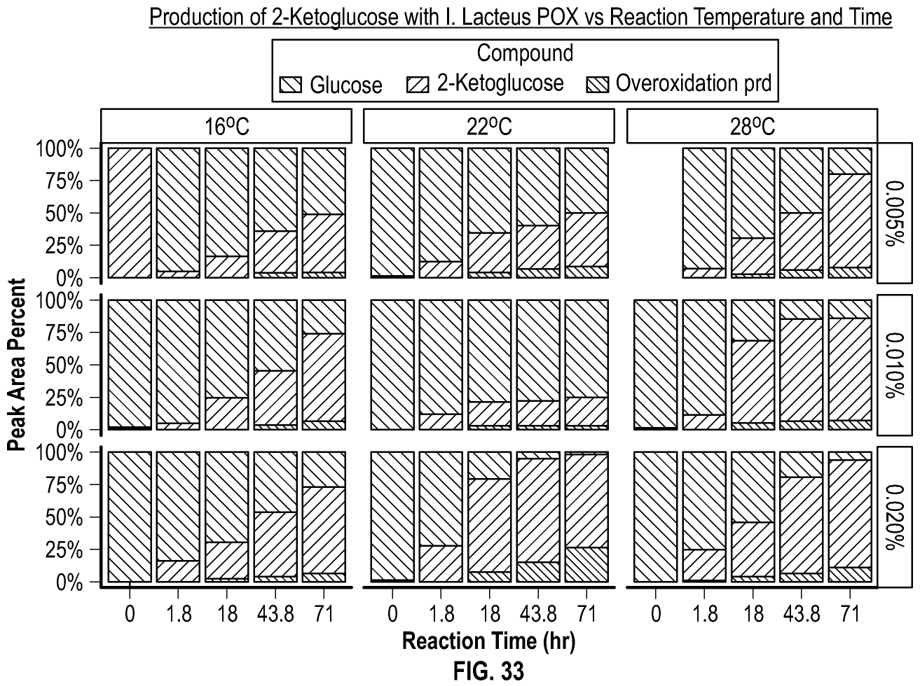

A 4 mL reaction was conducted in 20 ml vials placed in a vessel pressurized to 100 psi with O₂. The vials were charged with 20% w/v glucose, 100 mM potassium phosphate, pH 6, 0.005% catalase, and 0.005, 0.01, or 0.02% POX from Irpex lacteus. The reaction was stirred at 500 rpm at 16 or 28° C. At 71 hours, the reaction was stopped and analyzed by reverse-phase HPLC-MS. Production of 2-ketoglucose as the majority product (83% based on peak area) was confirmed for reactions at 28° C. and 0.020% with some formation of over oxidation products (11%) and unreacted glucose (6%), FIG. 33.

### Example 7

Production of 2-Ketogluconate from 2-Ketoglucose Using a Glucose Oxidase Catalyst

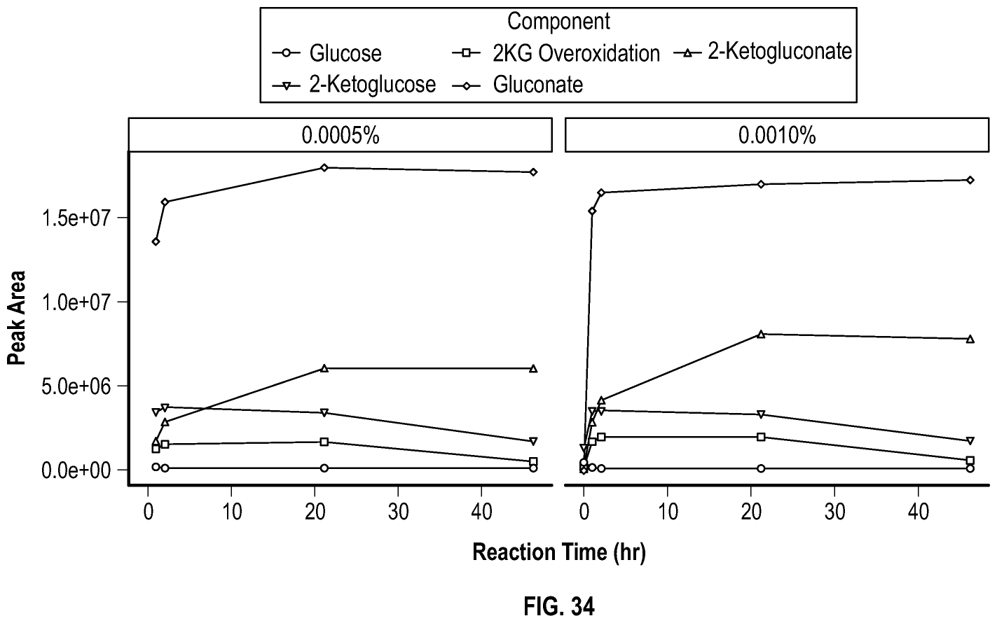

A 4 mL reaction was conducted in 20 ml vials placed in a vessel pressurized to 100 psi with O₂. The vials were charged with 2-ketoglucose generated as described in Example 6 and treated with tangential flow filtration to remove enzymes, 100 mM citrate buffer, pH 5, 0.0001% catalase, and either 0.0005 or 0.001% w/v GOX. The reaction pH was adjusted back to 5.0 with NaOH at each timepoint. 2-ketogluconate was produced as a major product with some gluconate produced from unreacted glucose in the POX reaction to 2-ketoglucose, unreacted 2-ketoglucose, and 2-ketoglucose over oxidation products present. The results are presented in FIG. 34.

### Example 8

Production of 2-Ketoglucodialdose from Glucodialdose Using a Pyranose Oxidase Catalyst

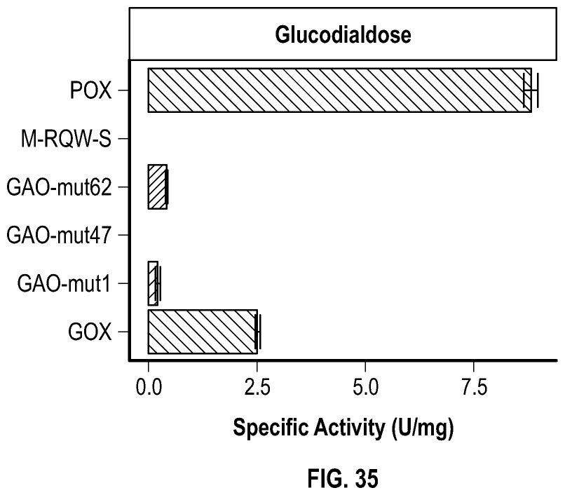

A colorimetric ABTS assay as described in Example 3 was conducted in a microtiter plate with each well containing 20 mM glucodialdose, 50 mM potassium phosphate buffer, pH 6, and 50 uL of a 1000× either 17.4 mg/L M-RQWS GAO, 25.5 mg/mL GAO-Mut1, 10.5mg/mL GAO-mut47, 10.7 mg/mL GAO-mut62, 42 mg/mL GOX, or 30 mg/mL POX. POX activity was detected on glucodialdose with a specific activity >7.5 U/mg. The results are presented in FIG. 35.

### Example 9

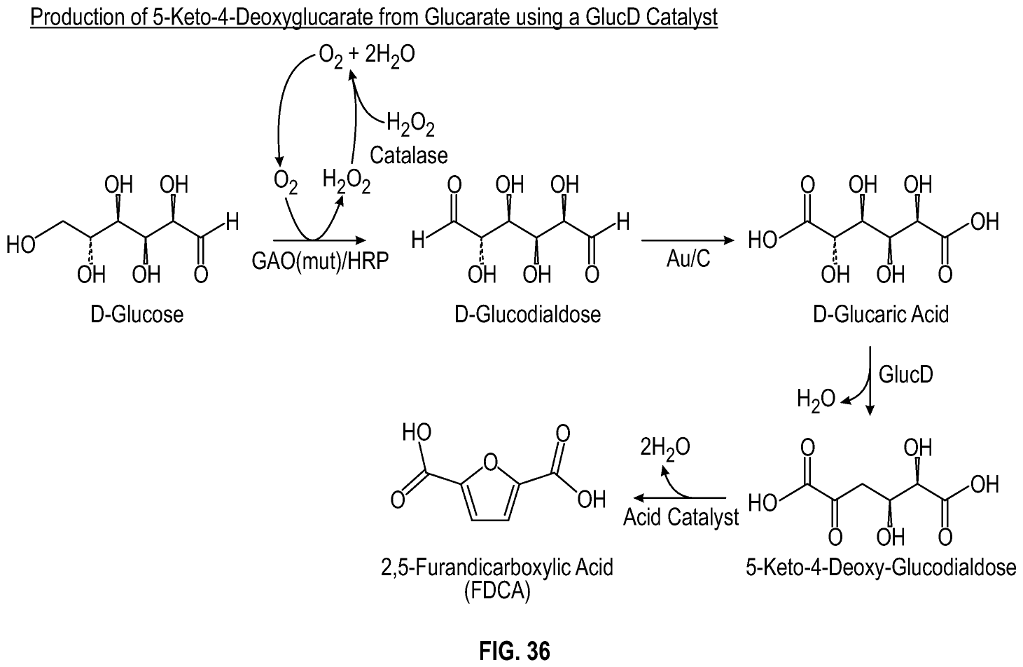

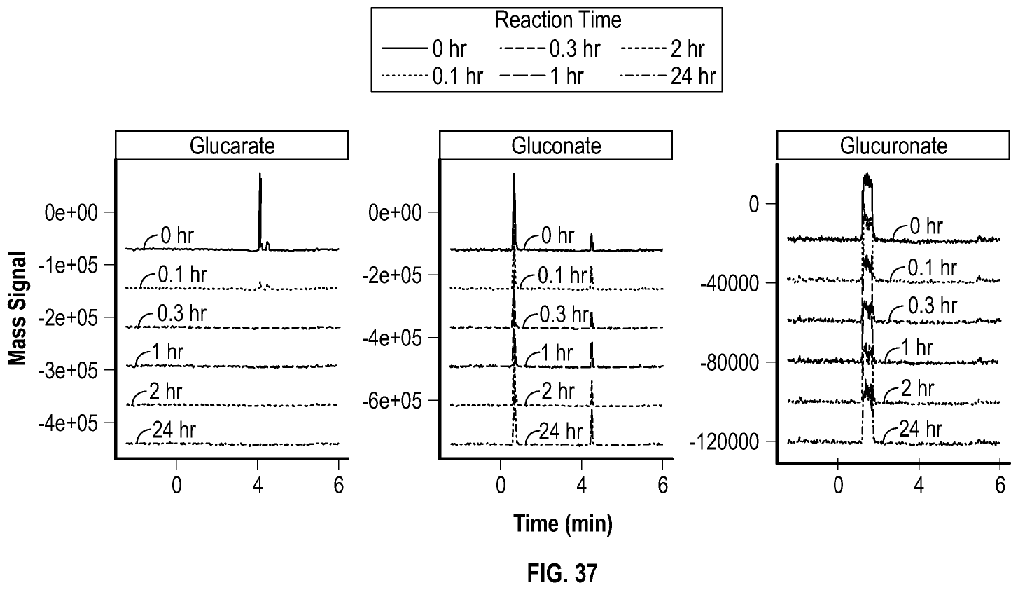

A 100 μL volume was prepared containing 0.01% w/v Escherichia coli glucarate dehydratase (GlucD), 10 mM substrate (glucarate, gluconate, or glucuronate), 50 mM potassium phosphate, pH 7.5, 5 mM MgSO₄, and 100 mM NaCl. The reaction was allowed to proceed for 24 hours and consumption of glucaric acid was monitored via LCMS at 0, 5mins, 20 mins, 1 hr, 2 hrs, and 24 hours. Full conversion of glucarate was observed within 20 minutes. No reaction was observed when gluconic or glucuronic acid was used as a substrate. The reaction is schematized in FIG. 36 and the results are graphed in Figure

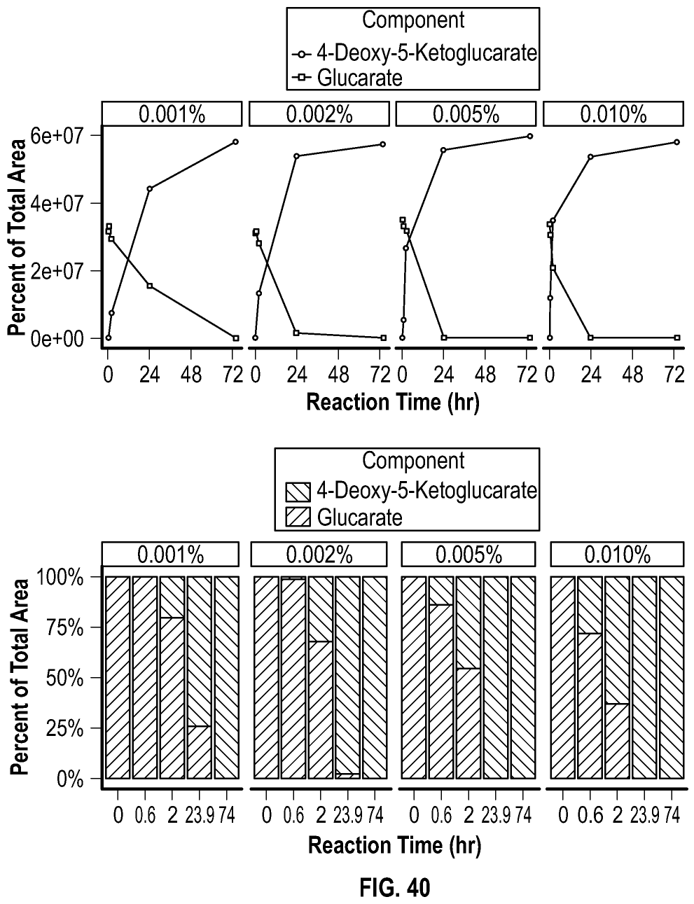

To determine optimal enzyme loading, a 100 μL volume was prepared containing 0.001, 0.002, 0.005, or 0.01% w/v glucarate dehydratase (GlucD), 10% w/v glucarate, 50 mM potassium phosphate, pH 7.5, 5 mM MgSO₄, and 100 mM NaCl. The reaction was allowed to proceed for 74 hours and formation of 4-deoxy-5-keoglucarate and consumption of glucarate was monitored via reverse phase LCMS. Full conversion was achieved with all concentrations of GlucD tested after 72 hours with full conversion occurring within 24 hrs using 0.005% enzyme. The reaction is schematized in FIG. 36 and the results are graphed in FIG. 40.

### Example 10

Production of Erythorbic Acid from Glucose

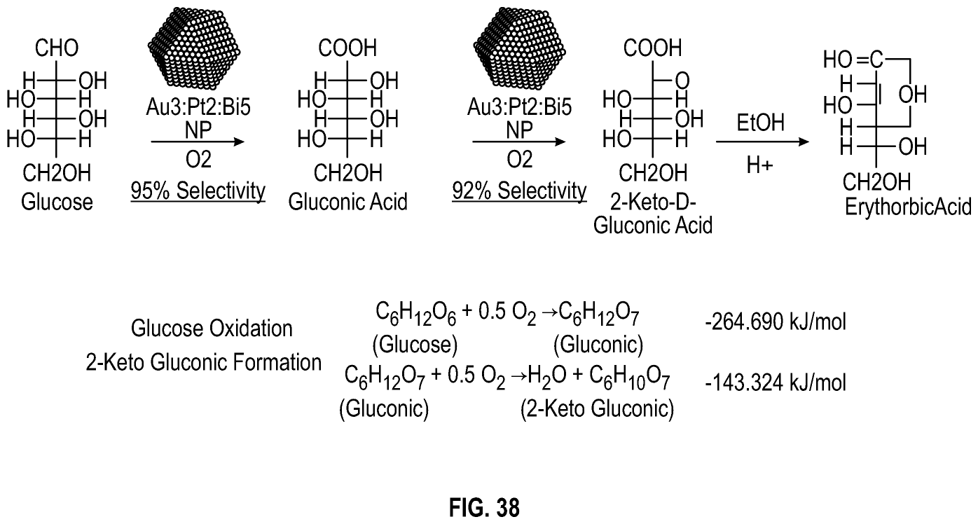

A method for production of D-erythorbic acid (D-EA) is depicted in FIG. 38. D-EA is also known as D-(-)-isoascorbic acid, araboascorbic acid, glucosaccharonic acid, erycorbin, D-isoascorbic acid, saccharosonic acid, mercate 5, neo-cebicure, D-araboascorbic acid, NSC 8117, and D-erythro-hex-2-enoic acid γ-lactone) is a stereoisomer of ascorbic acid (vitamin C) commonly used as a food preservative to prevent oxidation (browning) and formation of nitrosamines during cooking or curing. For example. D-EA is a common preservative in cured meats and frozen vegetables. The compound is also an effective enhancer of nonheme-iron absorption.

With reference to FIG. 38, glucose can be oxidized in the presence of a TMC and an oxidizing agent (O₂) under conditions suitable for the formation of gluconic acid. The oxidation of glucose to gluconic acid may have a selectivity of equal to or greater than about 85%, additionally or alternatively equal to or greater than about 90%, or additionally or alternatively equal to or greater than about 95%. Gluconic acid may be further oxidized to form 2-keto-D-gluconic acid in the presence of a TMC and an oxidizing agent (O₂) with a reaction selectivity of may have a selectivity of equal to or greater than about 80%, additionally or alternatively equal to or greater than about 85%, or additionally or alternatively equal to or greater than about 90%. The 2-keto gluconic acid may then be lactonized in an alcohol solvent (e.g., ethanol) and an acid catalyst to form erythorbic acid. The stochiometric reactions for the formation of gluconic acid from glucose and 2-ketogluconic acid from gluconic acid are also presented in FIG. 38. According to the disclosed methods, vitamin C production is more cheaply and more efficiently produced via a chemoenzymatic process or a catalytic process rather than a fermentation process. 1. Using a membrane system (30 Kda or below) which retains enzymes and avoids directly using cells. 2. Selective oxidation of L-Sorbose/Fructose using the Solugen Mesoporous AuPt Catalyst. 3. Selective oxidation of Gluconic acid and Gulonic acid into 2-keto gluconic acid and 2-keto gulonic acid.

### Example 11

Production of FDCA from Potassium Glucarate

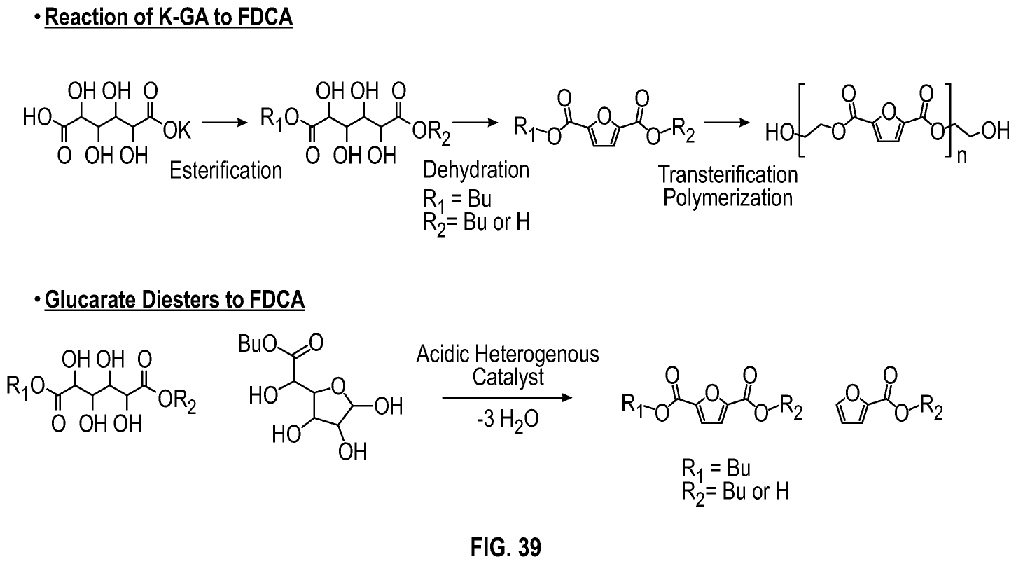

FDCA was produced using an acidic catalyst. The acidic catalyst investigated were zeolithes, H-ZSM5, MCM 41, sulfated zirconia, sulfonated silica, heteropolyacids. Catalyst powders, granulates, extrudates tablets. The samples were characterized by a BET of 20m²/g-100 m²/g and a crush strength of 60 N/cm². Selectivity >60%, conversion >60%, side products unknown. The reactions are presented in FIG. 39 and the results are presented in FIG. 40.

### Example 12

Generation of dehydrated D-glucohexodialdose (GDA) product from heat incubation. One-Step Parr Bomb Reaction with GAO-Mut47 to Produce D-Glucodialdose with high yield.

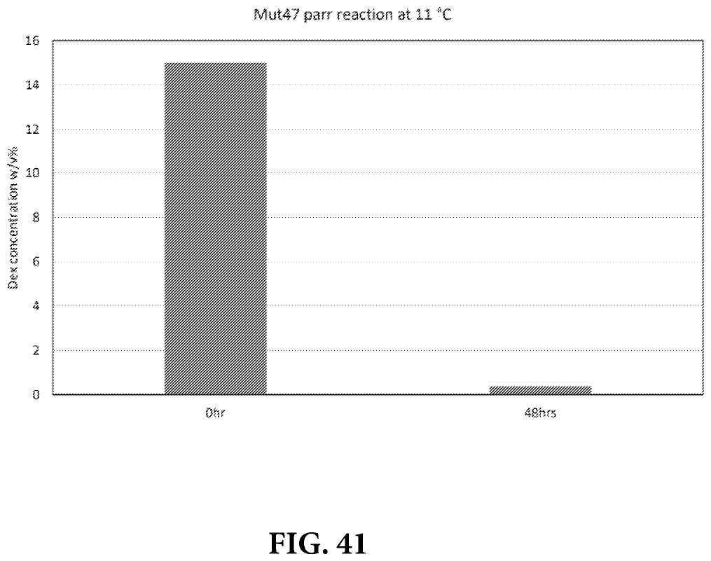

A 100 ml reaction was conducted in a 400 mL vessel pressurized to 100 psi. The vessel was charged with 50 mM sodium phosphate pH 8 buffer, 50 μM MnSO₄, 15 w/v % glucose, 0.005 w/v % catalase, 0.001% horseradish peroxidase, and 0.01% engineered GAO. The reaction was stirred 500 rpm at 11° C. for 48 hours. Samples were taken at 0and 48 hours then assayed with HPLC to measure residual glucose. After 48 hrs reaction, >95% of glucose was converted to GDA, the results are shown in FIG. 41. Heat incubation of GDA solution to catalyze the dehydration reaction

100 μL of GDA solution that was generated from the parr reaction (˜14.5 w/v % of GDA, 0.5% w/v % of glucose) was aliquoted into each well of the PCR plate, and the samples were heat incubated at a range of temperatures from 40 to 90° C. Sample were taken at time 0, 0.5, 1, 2, 4, 6, 8, and 24 hrs then assayed with HPLC to monitor the formation of dehydrated product, 1,2,3,4-tetrahydroxylbenzene, and other intermediate products

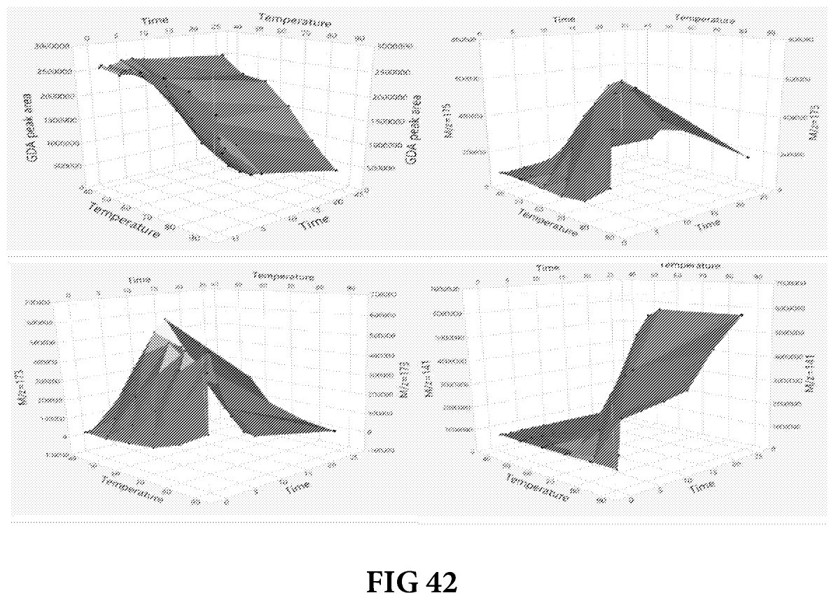

The results were analyzed by HPLC. As shown in FIG. 42, GDA degradation is observed when the incubation temperature is higher than 50° C., and the degradation percentage increases by increasing the incubation temperature or elongating the incubation time. After 24 hrs incubation at 90° C., the GDA degradation percentage is about 90%. With short incubation time around 10 hrs, two intermediate compounds with molecular weight of 176 and 174 were generated in the incubation reaction. After increasing the incubation time to 24 hrs, the reaction yielded the 1,2,3,4-tetrahydroxylbenzene (142 g/mol) as the major product.

### Example 13

Characterization of the Generated 1,2,3,4-tetrahydroxylbenzene.

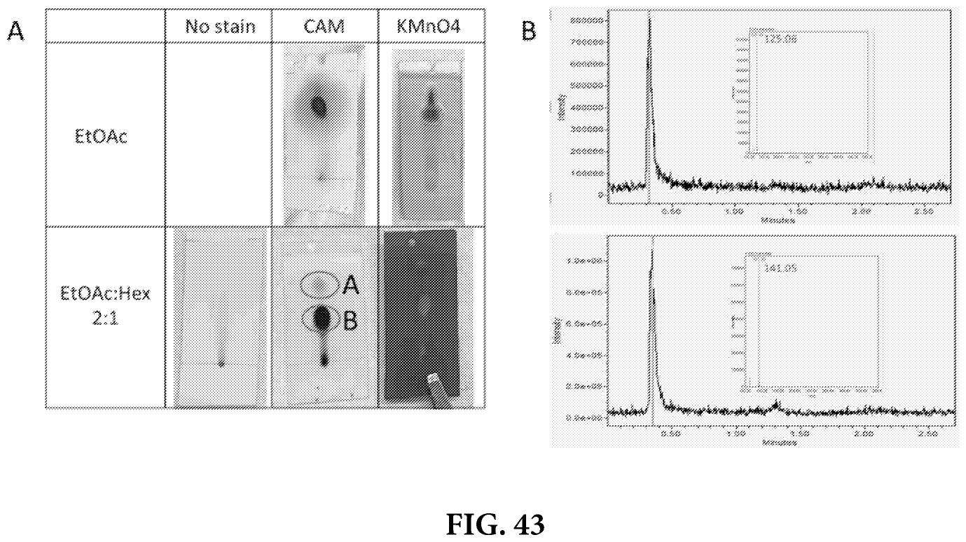

40mL of parr reaction product with 14.5 w/v % GDA and 0.5 w/v % of glucose were incubated at 90° C. for 30 hrs to convert the GDA to 1,2,3,4-tetrahydroxylbenzene. The incubated samples were extracted with 80 mL of ethyl acetate twice. The extracted samples were concentrated and run on TLC plates as shown in FIG. 43A. Two spots, A and B, were visualized on the plate. The fractions A and B were extracted from a preparative TLC plate and analyzed on LC/MS. The MS data, FIG. 43B, shows the molecular weight of fraction A and B are 126 and 142 respectively.

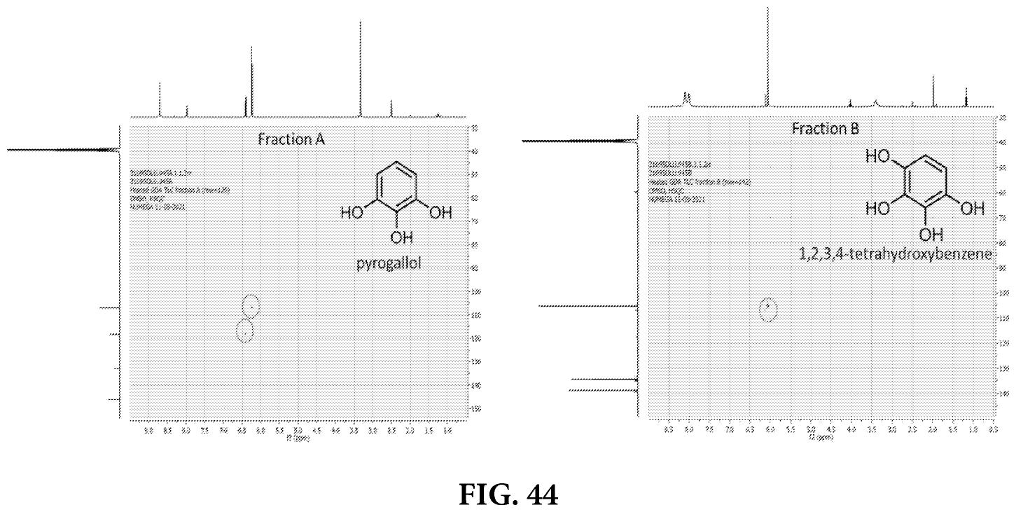

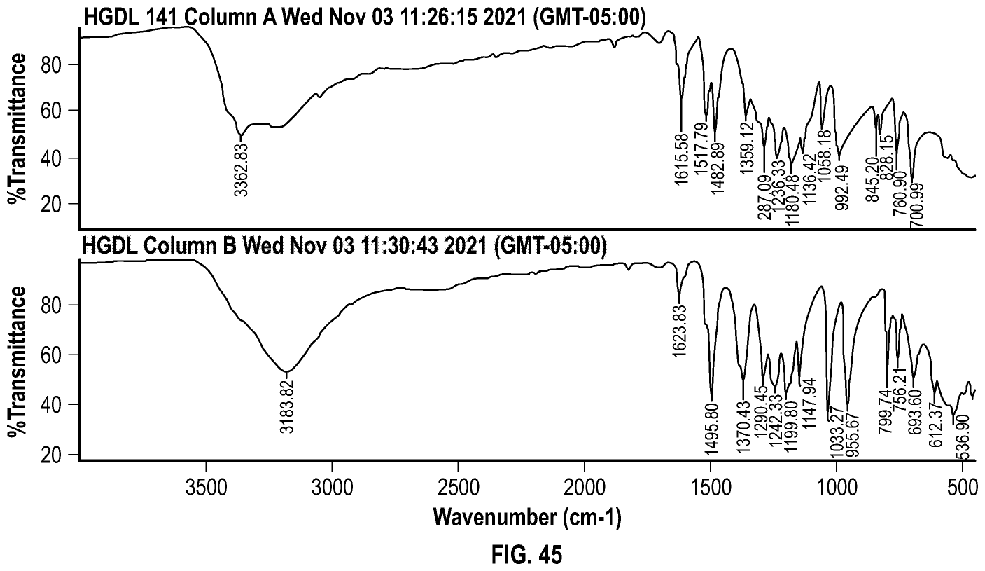

The purified fraction A and B were analyzed by 2D-NMR (HSQC, in DMSO-d6) to determine the molecular structures. The NMR data shown on FIG. 44 suggests the molecule in fraction A is 1,2,3-trihydroxybenzene, also called pyrogallol, and the molecule in fraction B is 1,2,3,4-tetrahydroxybenzene. FT-IR analysis, illustrated in FIG. 45, shows no carbonyl function group on either of the molecules which further confirms the proposed molecular structure from the NMR analysis.

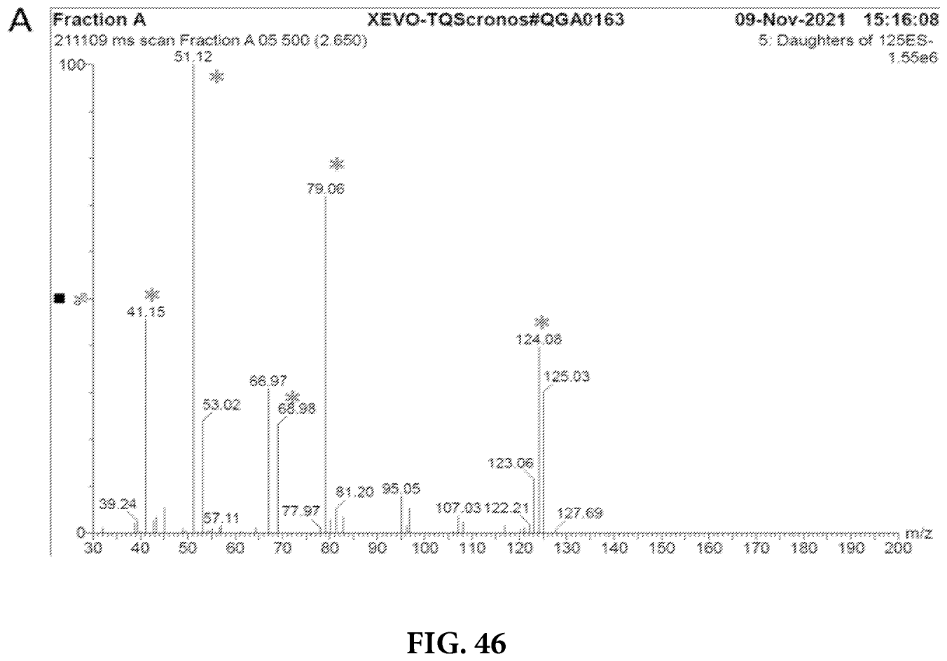

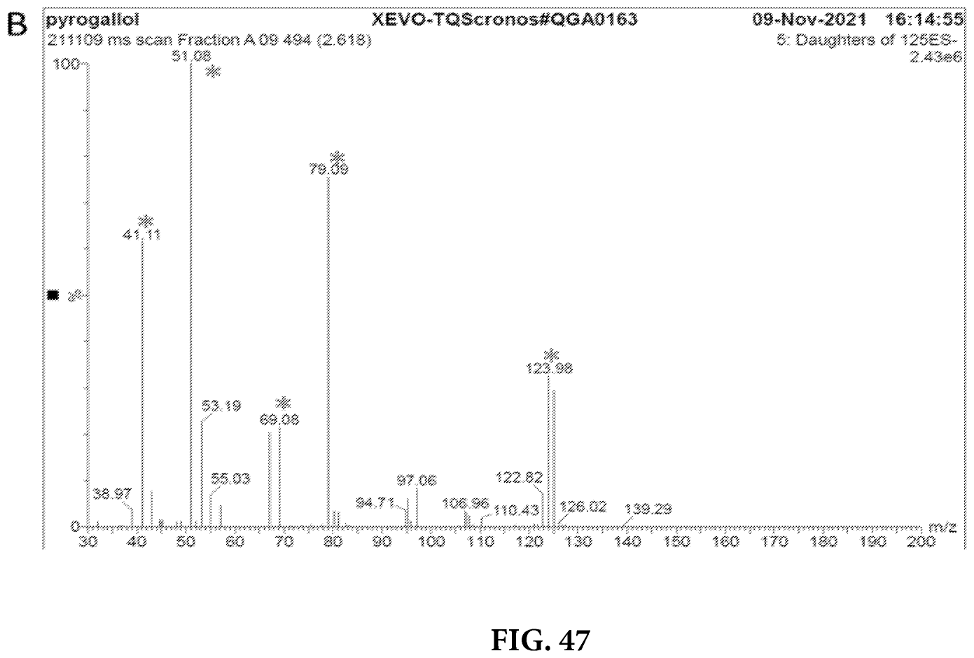

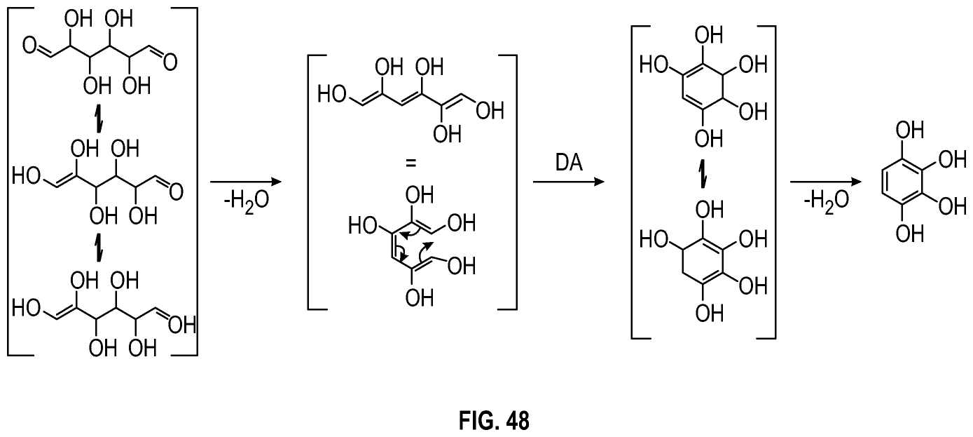

Further analysis was conducted with LC system that equipped with triple quadrupole mass detector and data is shown in FIGS. 46 and 47. The purified fraction A was analyzed with LC/QQQ and compared with the authentic standard pyrogallol. The fragmentation pattern of fraction A molecule perfectly match to the pyrogallol standard. A proposed reaction mechanism of pyrogallol and 1,2,3,4-tetrahydroxybenzene production from the GDA sample heat incubation is shown in FIG. 48.

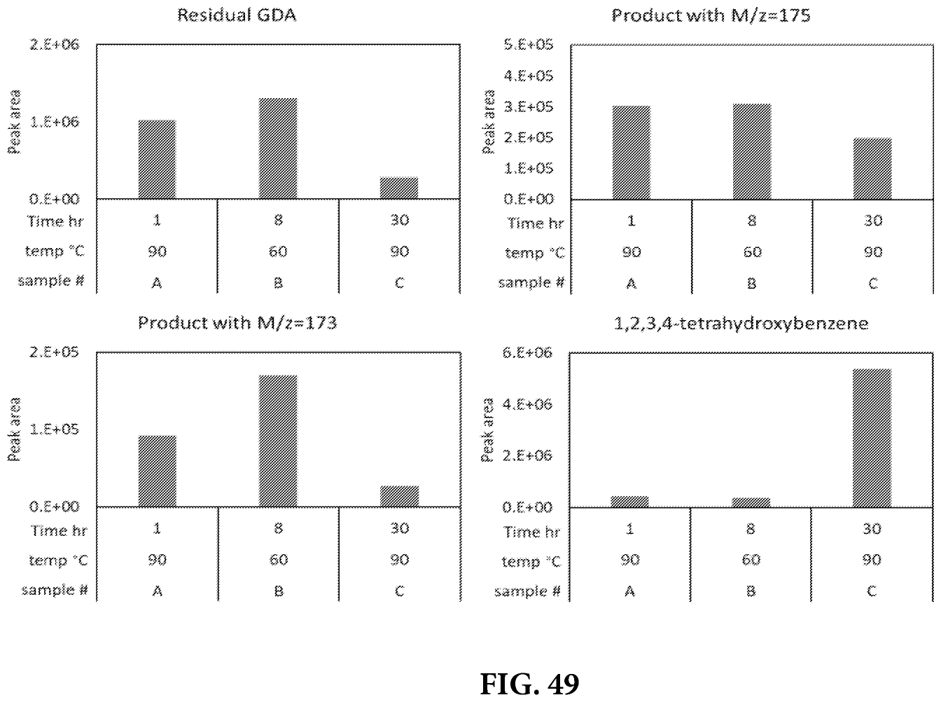

Three GDA samples generated from Parr reactions were incubated with different conditions, A) 90° C. for 1 hr, B) 60° C. for 8 hrs, or C) 90° C. for 30 hrs to maximize the conversion of GDA to either of two intermediate molecules or 1,2,3,4-tetrahydroxybenzene. As seen in FIG. 49, sample A has a relatively higher concentration of intermediate molecule with molecular weight of 176, and sample B has the highest concentration of the second of intermediate molecule with molecular weight of 174, and sample C has the highest conversion to 1,2,3,4-tetrahydroxybenzene.

All three different heat incubated GDA samples were tested as antimicrobial agents with several different industrially relevant bacteria. The General Aerobic Bacteria isolated and used for this testing were identified as Pseudomonas aeruginosa and Enterobacter aggolomerans. Wild strain Sulfate Reducing Bacteria and Acid Producing Bacteria isolated from the produced fluids was also used. They were placed in a microtiter plate, where a high throughput test can be performed to determine MIC (minimum inhibitor concentration). Samples were tested at the following doses (1000, 500, 250, 125, 62.5, 31.2, 15.6, 7.8, 3.9, and 1.95ppm) and concentration was calculated based on the initial GDA concentration (15 w/v %).

The antimicrobial testing results shown in FIG. 49 summarizes the MIC of each sample for different bacteria. The MIC is determined after 24 hours of incubation for the bacterial plates. The concentration of the well that displays no growth after the appropriate incubation time is recorded as the MIC value for the specific formulation. Sample C which has highest concentration of 1,2,3,4-tetrahydroxybenzene has significantly lower MIC for all three bacteria tested, which suggests 1,2,3,4-tetrahydroxybenzene has good antimicrobial activity.

TABLE V
Antimicrobial testing of three heat GDA samples.
Microtiter Study of antimicrobial activity

| | General Wild | | SRB (Sulfate | | APB (Acid | |
|---|---|---|---|---|---|---|
| Biocide | Strain Bacteria | | Reducing Bacteria) | | Producing Bacteria) | |
| Heat GDA A | 10,000 | ppm | 10,000 | ppm | 10,000 | ppm |
| Heat GDA B | 10,000 | ppm | 10,000 | ppm | 10,000 | ppm |
| Heat GDA C | 2500 | ppm | 2500 | ppm | 1250 | ppm |
| Glutaraldehyde - 50% | 1000 | ppm | 500 | ppm | 500 | ppm |

### ADDITIONAL DISCLOSURE

The following enumerated aspects of the present disclosures are provided as non-limiting examples.

A first aspect which is a molecular manufacturing process comprising contacting a platform molecule with (i) a biocatalyst and (ii) a chemical catalyst under conditions suitable to produce a value-added chemical.

A second aspect which is the process of the first aspect wherein the platform chemical comprises glucose and the value-added chemical comprises glucaric acid.

A third aspect which is the process of any of the first through second aspects wherein the biocatalyst comprises galactose oxidase and the chemical catalyst comprises a transition metal catalyst.

A fourth aspect which is the process of any of the first aspect wherein the platform chemical comprises glucose and the value-added chemical comprises L-ascorbic acid.

A fifth aspect which is the process of the first aspect wherein the platform chemical comprises glucose and the value-added chemical comprises succinic acid.

A sixth aspect which is the process of the first aspect wherein the platform chemical comprises glucose and the value-added chemical comprises 2,5-furan dicarboxylic acid.

A seventh aspect which is the process of the first aspect wherein the platform chemical comprises glucose and the value-added chemical comprises 2,5-furan dicarboxylic acid dimethyl ester.

An eighth aspect which is the process of the first aspect wherein the platform chemical comprises ethanol and the value-added chemical comprises acetaldehyde.

A ninth aspect which is the process of the first aspect wherein the platform chemical comprises ethanol and the value-added chemical comprises propylene glycol.

A tenth aspect which is the process of the first aspect wherein the platform chemical comprises ethanol and the value-added chemical comprises lactic acid.

An eleventh aspect which is the process of the first aspect wherein the platform chemical comprises ethanol and the value-added chemical comprises acrylic acid.

A twelfth aspect which is the process of the first aspect wherein the platform chemical comprises ethanol and the value-added chemical comprises propanol.

A thirteenth aspect which is the process of the first aspect wherein the platform chemical comprises ethanol and the value-added chemical comprises acetoin.

A fourteenth aspect which is the process of the first aspect wherein the platform chemical comprises ethanol and the value-added chemical comprises 2,3-butanediol.

A fifteenth aspect which is the process of the first aspect wherein the platform chemical comprises ethanol and the value-added chemical comprises 1,3-butadiene.

A sixteenth aspect which is the process of the first aspect wherein the platform chemical comprises ethanol and the value-added chemical comprises 2-butanone.

A seventeenth aspect which is the process of the first aspect wherein the platform chemical comprises ethylene glycol and the value-added chemical comprises glycolic acid.

An eighteenth aspect which is the process of the first aspect wherein the platform chemical comprises ethylene glycol and the value-added chemical comprises ethanolamine.

A nineteenth aspect which is the process of the first aspect wherein the platform chemical comprises ethylene glycol and the value-added chemical comprises glycerol.

A twentieth aspect which is the process of the first aspect wherein the platform chemical comprises glycerol and the value-added chemical comprises dihydroxyacetone.

A twenty-first aspect which is the process of the first aspect wherein the platform chemical comprises 3-cyanopyridine and the value-added chemical comprises nicotinamide.

A twenty-second aspect which is the process of the first aspect wherein the value-added chemical has a purity of equal to or greater than about 80%.

A twenty-third aspect which is a molecular manufacturing process comprising:

contacting a platform molecule with a biocatalyst to produce an intermediate product; and contacting the intermediate product with a chemical catalyst under conditions suitable to produce a value-added chemical from the intermediate product.

A twenty-fourth aspect which is a molecular manufacturing process comprising contacting a platform molecule with a chemical catalyst to produce an intermediate product; and contacting the intermediate product with a biocatalyst catalyst under conditions suitable to produce a value-added chemical from the intermediate product.

A twenty-fifth aspect which is the process of any of the twenty-third through twenty-fourth aspects wherein at least one producing the intermediate product or producing the value-added chemical comprises causing at least one of an oxidation reaction, a dehydration reaction, a carboxylation reaction, or a hydrogenation reaction.

A twenty-sixth aspect which is the process of any one of the twenty-third through twenty-fifth aspects further comprising purifying the value-added chemical to produce a final product.

A twenty-seventh aspect which is the process of any one of the twenty-third through twenty-sixth aspects wherein the platform chemical comprises glucose and the value-added chemical comprises glucaric acid.

A twenty-eighth aspect which is the process of any of the twenty-third through twenty-sixth aspects wherein the biocatalyst comprises galactose oxidase and the chemical catalyst comprises a transition metal catalyst.

A twenty-ninth aspect which is the process of any of the twenty-third through twenty-eighth aspects wherein the platform chemical comprises glucose and the value-added chemical comprises L-ascorbic acid.

A thirtieth aspect which is the process of any of the twenty-third through twenty-ninth aspects wherein the platform chemical comprises glucose and the value-added chemical comprises succinic acid.

A thirty-first aspect which is the process of any of the twenty-third through thirtieth aspects wherein the platform chemical comprises glucose and the value-added chemical comprises 2,5-furan dicarboxylic acid.

A thirty-second aspect which is the process of any the twenty-third through thirty-first aspect wherein the platform chemical comprises glucose and the value-added chemical comprises 2,5-furan dicarboxylic acid dimethyl ester.

A thirty-third aspect which is the process of any of the twenty-third through thirty-second aspects wherein the platform chemical comprises ethanol and the value-added chemical comprises acetaldehyde.

A thirty-fourth aspect which is the process of any of the twenty-third through thirty-third aspects wherein the platform chemical comprises ethanol and the value-added chemical comprises propylene glycol.

A thirty-fifth aspect which is the process of any of the twenty-third through thirty-fourth aspects wherein the platform chemical comprises ethanol and the value-added chemical comprises lactic acid.

A thirty-sixth aspect which is the process of any of the twenty-third through thirty-fifth aspects wherein the platform chemical comprises ethanol and the value-added chemical comprises acrylic acid.

A thirty-seventh aspect which is the process of any of the twenty-third through thirty-sixth aspects wherein the platform chemical comprises ethanol and the value-added chemical comprises propanol.

A thirty-eighth aspect which is the process of any of the twenty-third through thirty-seventh aspects wherein the platform chemical comprises ethanol and the value-added chemical comprises acetoin.

A thirty-ninth aspect which is the process of any of the twenty-third through thirty-eighth aspects wherein the platform chemical comprises ethanol and the value-added chemical comprises 2,3-butanediol.

A fortieth aspect which is the process of any of the twenty-third through thirty-ninth aspects wherein the platform chemical comprises ethanol and the value-added chemical comprises 1,3-butadiene.

A forty-first aspect which is the process of any of the twenty-third through fortieth aspects wherein the platform chemical comprises ethanol and the value-added chemical comprises 2-butanone.

A forty-second aspect which is the process of any of the twenty-third through forty-first aspects wherein the platform chemical comprises ethylene glycol and the value-added chemical comprises glycolic acid.

A forty-third aspect which is the process of any of the twenty-third through forty-second aspects wherein the platform chemical comprises ethylene glycol and the value-added chemical comprises ethanolamine.

A forty-fourth aspect which is the process of any of the twenty-third through forty-third aspects wherein the platform chemical comprises ethylene glycol and the value-added chemical comprises glycerol.

A forty-fifth aspect which is the process of any of the twenty-third through forty-fourth aspects wherein the platform chemical comprises glycerol and the value-added chemical comprises dihydroxyacetone.

A forty-sixth aspect which is the process of any of the twenty-third through forty-fifth aspects wherein the platform chemical comprises 3-cyanopyridine and the value-added chemical comprises nicotinamide.

A forty-seventh aspect which is the process of any of the twenty-third through forty-sixth aspects wherein the value-added chemical has a purity of equal to or greater than about 80%.

The subject matter having been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A molecular manufacturing process comprising:
contacting a platform molecule with (i) a biocatalyst and (ii) a chemical catalyst under conditions suitable to produce a value-added chemical.

2. The process of claim 1, wherein the platform chemical comprises glucose and the value-added chemical comprises glucaric acid.

3. The process of claim 2, wherein the biocatalyst comprises galactose oxidase and the chemical catalyst comprises a transition metal catalyst.

4. The process of claim 1, wherein the platform chemical comprises glucose and the value-added chemical comprises L-ascorbic acid.

5. The process of claim 1, wherein the platform chemical comprises glucose and the value-added chemical comprises succinic acid.

6. The process of claim 1, wherein the platform chemical comprises glucose and the value-added chemical comprises 2,5-furan dicarboxylic acid.

7. The process of claim 1, wherein the platform chemical comprises glucose and the value-added chemical comprises 2,5-furan dicarboxylic acid dimethyl ester.

8. The process of claim 1, wherein the platform chemical comprises ethanol and the value-added chemical comprises acetaldehyde.

9. The process of claim 1, wherein the platform chemical comprises ethanol and the value-added chemical comprises propylene glycol.

10. The process of claim 1, wherein the platform chemical comprises ethanol and the value-added chemical comprises lactic acid.

11. The process of claim 1, wherein the platform chemical comprises ethanol and the value-added chemical comprises acrylic acid.

12. The process of claim 1, wherein the platform chemical comprises ethanol and the value-added chemical comprises propanol.

13. The process of claim 1, wherein the platform chemical comprises ethanol and the value-added chemical comprises acetoin.

14. The process of claim 1, wherein the platform chemical comprises ethanol and the value-added chemical comprises 2,3-butanediol.

15. The process of claim 1, wherein the platform chemical comprises ethanol and the value-added chemical comprises 1,3-butadiene.

16. The process of claim 1, wherein the platform chemical comprises ethanol and the value-added chemical comprises 2-butanone.

17. The process of claim 1, wherein the platform chemical comprises ethylene glycol and the value-added chemical comprises glycolic acid.

18. The process of claim 1, wherein the platform chemical comprises ethylene glycol and the value-added chemical comprises ethanolamine.

19. The process of claim 1, wherein the platform chemical comprises ethylene glycol and the value-added chemical comprises glycerol.

20. The process of claim 1, wherein the platform chemical comprises glycerol and the value-added chemical comprises dihydroxyacetone.


SEQUENCE LISTING (17 sequences)

SEQ ID NO: 1 - protein, 1917 aa, synthetic construct
ALASERALAPROILEGLYSERALAILEPROARGASNASNTRPALAVALTHRCYSASPSERALAGLNSERGLYASNGLUCYSASNLYSALAILEASPGLYASNLYSASPTHRPHETRPHISTHRPHETYRGLYALAASNGLYASPPROLYSPROPROHISTHRTYRTHRILEASPMETLYSTHRTHRGLNASNVALASNGLYLEUSERVALLEUPROARGGLNASPGLYASNGLNASNGLYTRPILEGLYARGHISGLUVALTYRLEUSERSERASPGLYTHRASNTRPGLYSERPROVALALASERGLYSERTRPPHEALAASPSERTHRTHRLYSTYRSERASNPHEGLUTHRARGPROALAARGTYRVALARGLEUVALALAILETHRGLUALAASNGLYGLNPROTRPTHRSERILEALAGLUILEASNVALPHEGLNALASERSERTYRTHRALAPROGLNPROGLYLEUGLYARGTRPGLYPROTHRILEASPLEUPROILEVALPROALAALAALAALAILEGLUPROTHRSERGLYARGVALLEUMETTRPSERSERTYRARGASNASPALAPHEGLUGLYSERPROGLYGLYILETHRLEUTHRSERSERTRPASPPROSERTHRGLYILEVALSERASPARGTHRVALTHRVALTHRLYSHISASPMETPHECYSPROGLYILESERMETASPGLYASNGLYGLNILEVALVALTHRGLYGLYASNASPALALYSLYSTHRSERLEUTYRASPSERSERSERASPSERTRPILEPROGLYPROASPMETGLNVALALAARGGLYTYRGLNSERSERALATHRMETSERASPGLYARGVALPHETHRILEGLYGLYSERPHESERGLYGLYVALPHEGLULYSASNGLYGLUVALTYRSERPROSERSERLYSTHRTRPTHRSERLEUPROASNALALYSVALASNPROMETLEUTHRALAASPLYSGLNGLYLEUTYRLYSSERASPASNHISALATRPLEUPHEGLYTRPLYSLYSGLYSERVALPHEGLNALAGLYPROSERTHRALAMETASNTRPTYRTYRTHRSERGLYSERGLYASPVALLYSSERALAGLYLYSARGGLNSERASNARGGLYVALALAPROASPALAMETSERGLYASNALAVALMETTYRASPALAVALLYSGLYLYSILELEUTHRPHEGLYGLYSERPROASPTYRTHRASPSERASPALATHRTHRASNALAHISILEILETHRLEUGLYGLUPROGLYTHRSERPROASNTHRVALPHEALASERASNGLYLEUTYRPHEALAARGTHRPHEHISTHRSERVALVALLEUPROASPGLYSERTHRPHEILETHRGLYGLYGLNARGARGGLYILEPROPHEGLUASPSERTHRPROVALPHETHRPROGLUILETYRVALPROGLUGLNASPTHRPHETYRLYSGLNASNPROASNSERILEVALARGALATYRHISSERILESERLEULEULEUPROASPGLYARGVALPHEASNGLYGLYGLYGLYLEUCYSGLYASPCYSTHRTHRASNHISPHEASPALAGLNILEPHETHRPROASNTYRLEUTYRASPSERASNGLYASNLEUALATHRARGPROLYSILETHRARGTHRSERTHRGLNSERVALLYSVALGLYGLYARGILETHRILESERTHRASPSERSERILESERLYSALASERLEUILEARGTYRGLYTHRALATHRHISTHRVALASNTHRASPGLNARGARGILEPROLEUTHRLEUTHRASNASNGLYGLYASNSERTYRSERPHEGLNVALPROSERASPSERGLYVALALALEUPROGLYTYRTRPMETLEUPHEVALMETASNSERALAGLYVALPROSERVALALASERTHRILEARGVALTHRGLN

SEQ ID NO: 2 - protein, 1869 aa, synthetic construct
ALASERALAPROILEGLYSERALAILEPROARGASNASNTRPALAVALTHRCYSASPSERALAGLNSERGLYASNGLUCYSASNLYSALAILEASPGLYASNLYSASPTHRPHETRPHISTHRPHETYRGLYALAASNGLYASPPROLYSPROPROHISTHRTYRTHRILEASPMETLYSTHRTHRGLNASNVALASNGLYLEUSERVALLEUPROARGGLNASPGLYASNGLNASNGLYTRPILEGLYARGHISGLUVALTYRLEUSERSERASPGLYTHRASNTRPGLYSERPROVALALASERGLYSERTRPPHEALAASPSERTHRTHRLYSTYRSERASNPHEGLUTHRARGPROALAARGTYRVALARGLEUVALALAILETHRGLUALAASNGLYGLNPROTRPTHRSERILEALAGLUILEASNVALPHEGLNALASERSERTYRTHRALAPROGLNPROGLYLEUGLYARGTRPGLYPROTHRILEASPLEUPROILEVALPROALAALAALAALAILEGLUPROTHRSERGLYARGVALLEUMETTRPSERSERTYRARGASNASPALAPHEGLUGLYSERPROGLYGLYILETHRLEUTHRSERSERTRPASPPROSERTHRGLYILEVALSERASPARGTHRVALTHRVALTHRLYSHISASPMETPHECYSPROGLYILESERMETASPGLYASNGLYGLNILEVALVALTHRGLYGLYASNASPALALYSLYSTHRSERLEUTYRASPSERSERSERASPSERTRPILEPROGLYPROASPMETGLNVALALAARGGLYTYRGLNSERSERALATHRMETSERASPGLYARGVALPHETHRILEGLYGLYSERPHESERGLYGLYVALPHEGLULYSASNGLYGLUVALTYRSERPROSERSERLYSTHRTRPTHRSERLEUPROASNALALYSVALASNPROMETLEUTHRALAASPLYSGLNGLYLEUTYRLYSSERASPASNHISALATRPLEUPHEGLYTRPLYSLYSGLYSERVALPHEGLNALAGLYPROSERTHRALAMETASNTRPTYRTYRTHRSERGLYSERGLYASPVALLYSSERALAGLYLYSARGGLNSERASNARGGLYVALALAPROASPALAMETSERGLYASNALAVALMETTYRASPALAVALLYSGLYLYSILELEUTHRPHEGLYGLYSERPROASPPHEGLUASPSERASPALATHRTHRASNALAHISILEILETHRLEUGLYGLUPROGLYTHRSERPROASNTHRVALPHEALASERASNGLYLEUTYRPHEALAARGTHRPHEHISTHRSERVALVALLEUPROASPGLYSERTHRPHEILETHRGLYGLYGLNARGARGGLYILEPROPHEGLUASPSERTHRPROVALPHETHRPROGLUILETYRVALPROGLUGLNASPTHRPHETYRLYSGLNASNPROASNSERILEVALARGALATYRHISGLYLEUCYSGLYASPCYSTHRTHRASNHISPHEASPALAGLNILEPHETHRPROASNTYRLEUTYRASPSERASNGLYASNLEUALATHRARGPROLYSILETHRARGTHRSERTHRGLNSERVALLYSVALGLYGLYARGILETHRILESERTHRASPSERSERILESERLYSALASERLEUILEARGTYRGLYTHRALATHRHISTHRVALASNTHRASPGLNARGARGILEPROLEUTHRLEUTHRASNASNGLYGLYASNSERTYRSERPHEGLNVALPROSERASPSERGLYVALALALEUPROGLYTYRTRPMETLEUPHEVALMETASNSERALAGLYVALPROSERVALALASERTHRILEARGVALTHRGLN

SEQ ID NO: 3 - protein, 1917 aa, synthetic construct
ALASERALAPROILEGLYSERALAILEPROARGASNASNTRPALAVALTHRCYSASPSERALAGLNSERGLYASNGLUCYSASNLYSALAILEASPGLYASNLYSASPTHRPHETRPHISTHRPHETYRGLYALAASNGLYASPPROLYSPROPROHISTHRTYRTHRILEASPMETLYSTHRTHRGLNASNVALASNGLYLEUSERVALLEUPROARGGLNASPGLYASNGLNASNGLYTRPILEGLYARGHISGLUVALTYRLEUSERSERASPGLYTHRASNTRPGLYSERPROVALALASERGLYSERTRPPHEALAASPSERTHRTHRLYSTYRSERASNPHEGLUTHRARGPROALAARGTYRVALARGLEUVALALAILETHRGLUALAASNGLYGLNPROTRPTHRSERILEALAGLUILEASNVALPHEGLNALASERSERTYRTHRALAPROGLNPROGLYLEUGLYARGTRPGLYPROTHRILEASPLEUPROILEVALPROVALALAALAALAILEGLUPROTHRSERGLYARGVALLEUMETTRPSERSERTYRARGASNASPARGPHEGLUGLYSERPROGLYGLYILETHRLEUTHRSERSERTRPASPPROSERTHRGLYILEVALSERASPARGTHRVALTHRVALTHRLYSHISASPMETPHECYSPROGLYILESERMETASPGLYASNGLYGLNILEVALVALTHRGLYGLYASNASPALALYSLYSTHRSERLEUTYRASPSERSERSERASPSERTRPILEPROGLYPROASPMETGLNVALALAARGGLYTYRGLNSERSERALATHRMETSERASPGLYARGVALPHETHRILEGLYGLYSERPHESERGLYGLYVALPHEGLULYSASNGLYGLUVALTYRSERPROSERSERLYSTHRTRPTHRSERLEUPROASNALALYSVALASNPROMETLEUTHRALAASPLYSGLNGLYLEUTYRLYSSERASPASNHISALATRPLEUPHEGLYTRPLYSLYSGLYSERVALPHEGLNALAGLYPROSERTHRALAMETASNTRPTYRTYRTHRSERGLYSERGLYASPVALLYSSERALAGLYLYSARGGLNSERASNARGGLYVALALAPROASPALAMETSERGLYASNALAVALMETTYRASPALAVALLYSGLYLYSILELEUTHRPHEGLYGLYSERPROHISPHEGLUASPSERASPALATHRTHRASNALAHISILEILETHRLEUGLYGLUPROGLYTHRSERPROASNTHRVALPHEALASERASNGLYLEUTYRPHEALAARGTHRTYRHISTHRSERVALVALLEUPROASPGLYSERTHRPHEILETHRGLYGLYGLNARGARGGLYILEPROPHEGLUASPSERTHRPROVALPHETHRPROGLUILETYRVALPROGLUGLNASPTHRPHETYRLYSGLNASNPROASNSERILEVALARGALATYRHISSERILESERLEULEULEUPROASPGLYARGVALPHEASNGLYGLYGLYGLYLEUCYSGLYASPCYSTHRTHRASNHISPHEASPALAGLNILEPHETHRPROASNTYRLEUTYRASPSERASNGLYASNLEUALATHRARGPROLYSILETHRARGTHRSERTHRGLNSERVALLYSVALGLYGLYARGILETHRILESERTHRASPSERSERILESERLYSALASERLEUILEARGTYRGLYTHRALATHRHISTHRVALASNTHRASPGLNARGARGILEPROLEUTHRLEUTHRASNASNGLYGLYASNSERTYRSERPHEGLNVALPROSERASPSERGLYVALALALEUPROGLYTYRTRPMETLEUPHEVALMETASNSERALAGLYVALPROSERVALALASERTHRILEARGVALTHRGLN

SEQ ID NO: 4 - protein, 1917 aa, synthetic construct
ALASERALAPROILEGLYSERALAILEPROARGASNASNTRPALAVALTHRCYSASPSERALAGLNSERGLYASNGLUCYSASNLYSALAILEASPGLYASNLYSASPTHRPHETRPHISTHRPHETYRGLYALAASNGLYASPPROLYSPROPROHISTHRTYRTHRILEASPMETLYSTHRTHRGLNASNVALSERGLYLEUSERVALLEUPROARGGLNASPGLYASNGLNASNGLYTRPILEGLYARGHISGLUVALTYRLEUSERSERASPGLYTHRASNTRPGLYSERPROVALALASERGLYSERTRPPHEALAASPSERTHRTHRLYSTYRSERASNPHEGLUTHRARGPROALAARGTYRVALARGLEUVALALAILETHRGLUALAASNGLYGLNPROTRPTHRSERILEALAGLUILEASNVALPHEGLNALASERSERTYRTHRALAPROGLNPROGLYLEUGLYARGTRPGLYPROTHRILEASPLEUPROILEVALPROVALALAALAALAILEGLUPROTHRSERGLYARGVALLEUMETTRPSERSERTRPARGASNASPALAPHEGLUGLYSERPROGLYGLYILETHRLEUTHRSERSERTRPASPPROSERTHRGLYILEVALSERASPARGTHRVALTHRVALTHRLYSHISASPMETPHECYSPROGLYILESERMETASPGLYASNGLYGLNILEVALVALTHRGLYGLYASNASPALALYSLYSTHRSERLEUTYRASPSERSERSERASPSERTRPILEPROGLYPROASPMETGLNVALALAARGGLYTYRGLNSERSERALATHRMETSERASPGLYARGVALPHETHRILEGLYGLYSERPHESERGLYGLYVALPHEGLULYSASNGLYGLUVALTYRSERPROSERSERLYSTHRTRPTHRSERLEUPROASNALALYSVALASNPROMETLEUTHRALAASPLYSGLNGLYLEUTYRLYSSERASPASNHISALATRPLEUPHEGLYTRPLYSLYSGLYSERVALPHEGLNALAGLYPROSERTHRALAMETASNTRPTYRTYRTHRSERGLYSERGLYASPVALLYSSERALAGLYLYSARGGLNSERASNARGGLYVALALAPROASPALAMETSERGLYASNALAVALMETTYRASPALAVALLYSGLYLYSILELEUTHRPHEGLYGLYSERPROASPTYRTHRASPSERASPALATHRTHRASNALAHISILEILETHRLEUGLYGLUPROGLYTHRSERPROASNTHRVALPHEALASERASNGLYLEUTYRPHEALAARGTHRPHEHISTHRSERVALVALLEUPROASPGLYSERTHRPHEILETHRGLYGLYGLNARGARGGLYILEPROPHEGLUASPSERTHRPROVALPHETHRPROGLUILETYRVALPROGLUGLNASPTHRPHETYRLYSGLNASNPROASNSERILEVALARGALATYRHISSERILESERLEULEULEUPROASPGLYARGVALPHEASNGLYGLYGLYGLYLEUCYSGLYASPCYSTHRTHRASNHISPHEASPALAGLNILEPHETHRPROASNTYRLEUTYRASPSERASNGLYASNLEUALATHRARGPROLYSILETHRARGTHRSERTHRGLNSERVALLYSVALGLYGLYARGILETHRILESERTHRASPSERSERILESERLYSALASERLEUILEARGTYRGLYTHRALATHRHISTHRVALASNTHRASPGLNARGARGILEPROLEUTHRLEUTHRASNASNGLYGLYASNSERTYRSERPHEGLNVALPROSERASPSERGLYVALALALEUPROGLYTYRTRPMETLEUPHEVALMETASNSERALAGLYVALPROSERVALALASERTHRILEARGVALTHRGLN

SEQ ID NO: 5 - protein, 1917 aa, synthetic construct
ALASERALAPROILEGLYSERALAILEPROARGASNASNTRPALAVALTHRCYSASPSERALAGLNSERGLYASNGLUCYSASNLYSALAILEASPGLYASNLYSASPTHRPHETRPHISTHRPHETYRGLYALAASNGLYASPPROLYSPROPROHISTHRTYRTHRILEASPMETLYSTHRTHRGLNASNVALSERGLYLEUSERVALLEUPROARGGLNASPGLYASNGLNASNGLYTRPILEGLYARGHISGLUVALTYRLEUSERSERASPGLYTHRASNTRPGLYSERPROVALALASERGLYSERTRPPHEALAASPSERTHRTHRLYSTYRSERASNPHEGLUTHRARGPROALAARGTYRVALARGLEUVALALAILETHRGLUALAASNGLYGLNPROTRPTHRSERILEALAGLUILEASNVALPHEGLNALASERSERTYRTHRALAPROGLNPROGLYLEUGLYARGTRPGLYPROTHRILEASPLEUPROILEVALPROVALALAALAALAILEGLUPROTHRSERGLYARGVALLEUMETTRPSERSERTRPARGASNASPALAPHEGLUGLYSERPROGLYGLYILETHRLEUTHRSERSERTRPASPPROSERTHRGLYILEVALSERASPARGTHRVALTHRVALTHRLYSHISASPMETPHECYSPROGLYILESERMETASPGLYASNGLYGLNILEVALVALTHRGLYGLYASNASPALALYSLYSTHRSERLEUTYRASPSERSERSERASPSERTRPILEPROGLYPROASPMETGLNVALALAARGGLYTYRGLNSERSERALATHRMETSERASPGLYARGVALPHETHRILEGLYGLYSERPHESERGLYGLYVALPHEGLULYSASNGLYGLUVALTYRSERPROSERALALYSTHRTRPTHRPHELEUPROASNALALYSVALASNPROMETLEUTHRALAASPLYSGLNGLYLEUTYRLYSARGASPASNHISALATRPLEUPHEGLYTRPLYSLYSGLYSERVALPHEGLNALAGLYPROSERTHRALAMETASNTRPTYRTYRTHRSERGLYSERGLYASPVALLYSSERALAGLYLYSARGGLNSERASNARGGLYVALASPPROASPALAMETSERGLYASNALAVALMETTYRASPALAVALLYSGLYLYSILELEUTHRPHEGLYGLYSERPROASPTYRTHRASPSERASPALATHRTHRASNALAHISILEILETHRLEUGLYGLUPROGLYTHRSERPROASNTHRVALPHEALASERASNGLYLEUTYRPHEALAARGTHRPHEHISTHRSERVALVALLEUPROASPGLYSERTHRPHEILETHRGLYGLYGLNARGARGGLYILEPROPHEGLUASPSERTHRPROVALPHETHRPROGLUILETYRVALPROGLUGLNASPTHRPHETYRLYSLEUASNPROASNSERILEVALARGALATYRHISSERILESERLEULEULEUPROASPGLYARGVALPHEASNGLYGLYGLYGLYLEUCYSGLYASPCYSTHRTHRASNHISPHEASPALAGLNILEPHETHRPROASNTYRLEUTYRASPSERASNGLYASNLEUALATHRARGPROLYSILETHRARGTHRSERTHRGLNSERVALLYSVALGLYGLYARGILETHRILESERTHRASPSERSERILESERLYSALASERLEUILEARGTYRGLYTHRALATHRHISTHRVALASNTHRASPGLNARGARGILEPROLEUTHRLEUTHRASNASNGLYGLYASNSERTYRSERPHEGLNVALPROSERASPSERGLYVALALALEUPROGLYTYRTRPMETLEUPHEVALMETASNSERALAGLYVALPROSERVALALASERTHRILEARGVALTHRGLN

SEQ ID NO: 6 - protein, 1914 aa, synthetic construct
ALASERALAPROILEGLYSERALAILEPROARGASNASNTRPALAVALTHRCYSASPSERALAGLNSERGLYASNGLUCYSASNLYSALAILEASPGLYASNLYSASPTHRPHETRPHISTHRPHETYRGLYALAASNGLYPROLYSPROPROHISTHRTYRTHRILEASPMETLYSTHRTHRGLNASNVALSERGLYLEUSERVALLEUPROARGGLNASPGLYASNGLNASNGLYTRPILEGLYARGHISGLUVALTYRLEUSERSERASPGLYTHRASNTRPGLYSERPROVALALASERGLYSERTRPPHEALAASPSERTHRTHRLYSTYRSERASNPHEGLUTHRARGPROALAARGTYRVALARGLEUVALALAILETHRGLUALAASNGLYGLNPROTRPTHRSERILEALAGLUILEASNVALPHEGLNALASERSERTYRTHRALAPROGLNPROGLYLEUGLYARGTRPGLYPROTHRILEASPLEUPROILEVALPROVALALAALAALAILEGLUPROTHRSERGLYARGVALLEUMETTRPSERSERTYRARGASNASPARGPHEGLUGLYSERPROGLYGLYILETHRLEUTHRSERSERTRPASPPROSERTHRGLYILEVALSERASPARGTHRVALTHRVALTHRLYSHISASPMETPHECYSPROGLYILESERMETASPGLYASNGLYGLNILEVALVALTHRGLYGLYASNASPALALYSLYSTHRSERLEUTYRASPSERSERSERASPSERTRPILEPROGLYPROASPMETGLNVALALAARGGLYTYRGLNSERSERALATHRMETSERASPGLYARGVALPHETHRILEGLYGLYSERPHESERGLYGLYVALPHEGLULYSASNGLYGLUVALTYRSERPROSERALALYSTHRTRPTHRPHELEUPROASNALALYSVALASNPROMETLEUTHRALAASPLYSGLNGLYLEUTYRLYSSERASPASNHISALATRPLEUPHEGLYTRPLYSLYSGLYSERVALPHEGLNALAGLYPROSERTHRALAMETASNTRPTYRTYRTHRSERGLYSERGLYASPVALLYSSERALAGLYLYSARGGLNSERASNARGGLYVALALAPROASPALAMETSERGLYASNALAVALMETTYRASPALAVALLYSGLYLYSILELEUTHRPHEGLYGLYSERPROHISPHEGLUASPSERASPALATHRTHRASNALAHISILEILETHRLEUGLYGLUPROGLYTHRSERPROASNTHRVALPHEALASERASNGLYLEUTYRPHEALAARGTHRTYRHISTHRSERVALVALLEUPROASPGLYSERTHRPHEILETHRGLYGLYGLNARGARGGLYILEPROPHEGLUASPSERTHRPROVALPHETHRPROGLUILETYRVALPROGLUGLNASPTHRPHETYRLYSGLNASNPROASNSERILEVALARGALATYRHISSERILESERLEULEULEUPROASPGLYARGVALPHEASNGLYGLYGLYGLYLEUCYSGLYASPCYSTHRTHRASNHISPHEASPALAGLNILEPHETHRPROASNTYRLEUTYRASPSERASNGLYASNLEUALATHRARGPROLYSILETHRARGTHRSERTHRGLNSERVALLYSVALGLYGLYARGILETHRILESERTHRASPSERSERILESERLYSALASERLEUILEARGTYRGLYTHRALATHRHISTHRVALASNTHRASPGLNARGARGILEPROLEUTHRLEUTHRASNASNGLYGLYASNSERTYRSERPHEGLNVALPROSERASPSERGLYVALALALEUPROGLYTYRTRPMETLEUPHEVALMETASNSERALAGLYVALPROSERVALALASERTHRILEARGVALTHRGLN

SEQ ID NO: 7 - protein, 1767 aa, Aspergillus niger
METGLNTHRLEULEUVALSERSERLEUVALVALSERLEUALAALAALALEUPROHISTYRILEARGSERASNGLYILEGLUALASERLEULEUTHRASPPROLYSASPVALSERGLYARGTHRVALASPTYRILEILEALAGLYGLYGLYLEUTHRGLYLEUTHRTHRALAALAARGLEUTHRGLUASNPROASNILESERVALLEUVALILEGLUSERGLYSERTYRGLUSERASPARGGLYPROILEILEGLUASPLEUASNALATYRGLYASPILEPHEGLYSERSERVALASPHISALATYRGLUTHRVALGLULEUALATHRASNASNGLNTHRALALEUILEARGSERGLYASNGLYLEUGLYGLYSERTHRLEUVALASNGLYGLYTHRTRPTHRARGPROHISLYSALAGLNVALASPSERTRPGLUTHRVALPHEGLYASNGLUGLYTRPASNTRPASPASNVALALAALATYRSERLEUGLNALAGLUARGALAARGALAPROASNALALYSGLNILEALAALAGLYHISTYRPHEASNALASERCYSHISGLYTHRASNGLYTHRVALHISALAGLYPROARGASPTHRGLYASPASPTYRSERPROILEVALLYSALALEUMETSERALAVALGLUASPARGGLYVALPROTHRLYSLYSASPPHEGLYCYSGLYASPPROHISGLYVALSERMETPHEPROASNTHRLEUHISGLUASPGLNVALARGSERASPALAALAARGGLUTRPLEULEUPROASNTYRGLNARGPROASNLEUGLNVALLEUTHRGLYGLNTYRVALGLYLYSVALLEULEUSERGLNASNGLYTHRTHRPROARGALAVALGLYVALGLUPHEGLYTHRHISLYSGLYASNTHRHISASNVALTYRALAGLUHISGLUVALLEULEUALAALAGLYSERALAVALSERPROTHRILELEUGLUTYRSERGLYILEGLYMETLYSSERILELEUGLUPROLEUGLYILEASPTHRVALVALASPLEUPROVALGLYLEUASNLEUGLNASPGLNTHRTHRALATHRVALARGSERARGILETHRSERALAGLYALAGLYGLNGLYGLNALAALATRPPHEALATHRPHEASNGLUTHRPHEGLYASPTYRSERGLULYSALAHISGLULEULEUASNTHRLYSLEUGLUGLNTRPALAGLUGLUALAVALALAARGGLYGLYPHEHISASNTHRTHRALALEULEUILEGLNTYRGLUASNTYRARGASPTRPILEVALASNHISASNVALALATYRSERGLULEUPHELEUASPTHRALAGLYVALALASERPHEASPVALTRPASPLEULEUPROPHETHRARGGLYTYRVALHISILELEUASPLYSASPPROTYRLEUHISHISPHEALATYRASPPROGLNTYRPHELEUASNGLULEUASPLEULEUGLYGLNALAALAALATHRGLNLEUALAARGASNILESERASNSERGLYALAMETGLNTHRTYRPHEALAGLYGLUTHRILEPROGLYASPASNLEUALATYRASPALAASPLEUSERALATRPTHRGLUTYRILEPROTYRHISPHEARGPROASNTYRHISGLYVALGLYTHRCYSSERMETMETPROLYSGLUMETGLYGLYVALVALASPASNALAALAARGVALGLNMETSERSERHISVALMETTHRVALPHETYRALAMETALALEULYSILESERASPALAILELEUGLUASPTYRALASERMETGLN

SEQ ID NO: 8 - protein, 1335 aa, Penicillium griseoroseum
METTYRARGTRPPHEASNTRPGLNPHEGLUVALTHRCYSGLNSERASPALATYRILEALAPROHISASNGLUHISALAALAALAGLUPHELEULYSGLUGLNTYRPROLYSSERSERHISILELYSVALVALGLYASNGLYHISGLYPHEGLYASNLEUTHRTHRCYSVALASPASNALALEUTHRGLULYSPROTHRTYRILEVALSERLEUTHRASNLEULYSLYSLEUHISILEASPLYSLYSASNLEUTHRVALTHRPHEGLYALAGLYTRPASPVALASPASPLEUILEGLNGLULEULYSALAASNASPLEUSERPHESERASNLEUGLYVALGLUARGVALGLNASNPHEVALGLYALAALASERTHRGLYTHRHISGLYSERGLYSERASPLEUGLYASNILEALATHRGLNILEILEGLYLEUASNALAGLUGLULEULYSALAPHEARGILESERLEUGLYALALEUGLYLEUILETHRGLULEUTHRILELYSVALGLNPROTHRGLNLEULEULYSLYSTHRTHRLYSVALLEUASNALATHRSERASPTYRSERLYSMETTYRASNGLULEUALAGLNLEUTYRLYSGLUHISASPARGMETTHRVALTRPGLYPROHISPHEASPTRPASNALALYSSERGLNSERTRPASPLEUGLUPROTHRTYRPHELEUSERTYRTRPGLUPROTHRASNTYRTHRGLYVALARGASNCYSTHRLEUASNTYRCYSALAASNGLYCYSGLYASPCYSLYSLYSGLUTYRILECYSTYRASPGLUVALTHRASPALAALASERCYSSERPROGLNGLYVALCYSSERARGGLYPHETYRALAGLUILEGLUHISPHELEUPROILEGLUTYRPHEALAGLUALAALATHRASNTYRTHRILEPHEGLNGLNGLYGLNTHRSERARGMETLYSALAPROTYRASNLYSGLNMETVALMETGLNHISARGSERLEULYSGLYASPASPTHRTYRLEUSERPROVALASNTHRTYRASNLEUGLYPROASPLEUSERGLYVALPHEGLYVALILEGLUILEASPTRPILEGLNGLUTYRASNASNPHETHRTHRLEUTRPGLNASNGLNGLULEUALAHISGLUPHELEUPROGLNPHEGLYGLUTHRTYRASNALAARGSERHISTRPASNLYSMETSERALAPROASNALATHRTYRTHRLEUGLULYSPHEPROLYSLEUPROGLUPHELEUALAILEGLNLYSARGGLNASPPROLYSCYSGLNPHEVALASNGLUPHELEUVALGLUGLNLEUGLYILETHRARGCYSALAASNTYRILESERVAL

SEQ ID NO: 9 - protein, 1437 aa, Penicillium griseoroseum
LEUSERPROLYSPROALAPHELEULEULEULEULEUHISALAVALPHEGLYSERALATYRARGTRPPHEASNTRPGLNPHEGLUVALTHRCYSGLNSERASPALATYRILEALAPROHISASNGLUHISALAALAALAGLUPHELEULYSGLUGLNTYRPROLYSSERSERHISILELYSVALVALGLYASNGLYHISGLYPHEGLYASNLEUTHRTHRCYSVALASPASNALALEUTHRGLULYSPROTHRTYRILEVALSERLEUTHRASNLEULYSLYSLEUHISILEASPLYSLYSASNLEUTHRVALTHRPHEGLYALAGLYTRPASPVALASPASPLEUILEGLNGLULEULYSALAASNASPLEUSERPHESERASNLEUGLYVALGLUARGVALGLNASNPHEVALGLYALAALASERTHRGLYTHRHISGLYSERGLYSERASPLEUGLYASNILEALATHRGLNILEILEGLYLEUARGVALLEUASPSERGLNGLYGLYLEUARGVALILEASNGLULYSHISASNALAGLUGLULEULYSALAPHEARGILESERLEUGLYALALEUGLYLEUILETHRGLULEUTHRILELYSVALGLNPROTHRGLNLEULEULYSLYSTHRTHRLYSVALLEUASNALATHRSERASPTYRSERLYSMETTYRASNGLULEUALAGLNLEUTYRLYSGLUHISASPARGMETTHRVALTRPGLYPROHISPHEASPTRPASNALALYSSERGLNSERTRPASPLEUGLUPROTHRTYRPHELEUSERTYRTRPGLUPROTHRASNTYRTHRGLYVALARGASNCYSTHRLEUASNTYRCYSALAASNGLYCYSGLYASPCYSLYSLYSGLUTYRILECYSTYRASPGLUVALTHRASPALAALASERCYSSERPROGLNGLYVALCYSSERARGGLYPHETYRALAGLUILEGLUHISPHELEUPROILEGLUTYRPHEALAGLUALAALATHRASNTYRTHRILEPHEGLNGLNGLYGLNTHRSERARGMETLYSALAPROTYRASNLYSGLNMETVALMETGLNHISARGSERLEULYSGLYASPASPTHRTYRLEUSERPROVALASNTHRTYRASNLEUGLYPROASPLEUSERGLYVALPHEGLYVALILEGLUILEASPTRPILEGLNGLUTYRASNASNPHETHRTHRLEUTRPGLNASNGLNGLULEUALAHISGLUPHELEUPROGLNPHEGLYGLUTHRTYRASNALAARGSERHISTRPASNLYSMETSERALAPROASNALATHRTYRTHRLEUGLULYSPHEPROLYSLEUPROGLUPHELEUALAILEGLNLYSARGGLNASPPROLYSCYSGLNPHEVALASNGLUPHELEUVALGLUGLNLEUGLYILETHRARGCYSALAASNTYRILESERVAL

SEQ ID NO: 10 - protein, 1584 aa, Pseudarthrobacter phenanthrenivorans
METSERGLYHISARGTYRPROALAALAVALASPVALALAILEVALGLYSERGLYPROTHRALASERALATYRALAARGILELEUSERGLUGLUALAPROGLYALATHRILEALAMETPHEGLUVALGLYPROTHRVALSERILEPROPROGLYALAHISVALLYSASNILEGLUASPPROGLUARGARGSERSERALAGLNARGALASERGLUGLYPROGLYALAGLYALAGLUTHRVALSERSERPROGLYALAVALLYSSERGLYGLUARGARGALAARGPROGLYTHRTYRLEULEUGLNASPGLYTYRALAPHEPROGLYGLUASPGLYMETPROVALALAALAMETSERSERASNVALGLYGLYMETALAALAHISTRPTHRALAALACYSPROARGPROGLYGLYLYSGLUARGILEPROPHELEUPROASPLEUGLUASPLEULEUASPASPALAASPARGLEULEUGLYVALTHRTHRHISALAPHEASPGLYALAPROPHESERASPLEUVALARGGLUARGLEUALAALAVALVALASPASNGLYARGGLYPROALAPHEARGVALGLNPROMETPROLEUALAVALHISARGARGGLUASPGLYGLYLEUVALTRPSERGLYSERASPVALVALMETGLYASPVALTHRARGGLUASNPROGLNPHEHISLEUPHEASPGLUSERLEUVALTHRARGVALLEUVALGLUASPGLYVALALAALAGLYVALGLUVALGLNASPARGARGTHRGLYGLYTHRHISGLNVALSERALAARGTYRVALVALVALGLYALAASPALALEUARGTHRPROGLNLEULEUTRPALASERGLYILEARGPROASPALALEUGLYARGTYRLEUASNASPGLNALAGLNVALVALPHEALASERARGLEUARGGLYVALTHRALAPROGLNGLYSERALAALAALAASPGLYALALEUSERGLUGLNSERGLYVALALATRPVALPROTYRTHRASPGLUALAPROPHEHISGLYGLNILEMETGLNLEUASPALASERPROVALPROLEUALAGLUASPASPPROVALVALPROGLYSERILEVALGLYLEUGLYLEUPHECYSALALYSASPLEUGLNARGGLUASPARGVALALAPHEASPASPGLYALAARGASPSERTYRGLYMETPROALAMETARGILEHISTYRARGLEUTHRGLUARGASPARGGLUVALLEUGLUARGALAARGGLNGLUILEVALARGLEUGLYLYSALAVALGLYGLUPROLEUASPGLUGLNPROPHEVALLEUPROPROGLYALASERLEUHISTYRGLNGLYTHRTHRARGMETALAARGTHRASPASPGLYGLUSERVALCYSSERPROASPSERGLUVALTRPGLNVALPROGLYLEUPHEVALALAGLYASNGLYVALILEPROTHRALATHRALACYSASNPROTHRLEUTHRALAVALALALEUALAVALARGGLYALAARGLYSVALALAGLULYSLEUASNSERSERLEULEUMETSERASNSERASPASNARGVALSERLYSGLYGLYSERGLYSERGLYHISHISHIS

SEQ ID NO: 11 - protein, 1958 aa, Irpex lacteus
METSERASNLEUPROPROHISGLUILEHISALALYSTHRGLYILEASNGLNPHEASPVALPHEILEALAGLYSERGLYPROILEGLYALATHRTYRALAARGLEULEUTHRARGHISGLYTYRASNVALILEMETTHRGLUILEGLYASPGLNGLUTHRARGVALPROALASERHISLYSLYSASNGLUILEGLUTYRGLNLYSASPILEASPARGPHEVALARGVALILEGLNGLYALALEUSERTHRVALSERVALPROPROALASERTHRVALILEPROGLNLEUASPPROSERALATRPARGPROGLUASPPROSERGLNMETTHRLEULEUASNGLYARGASNPROASNGLNGLNTHRTYRASPASNLEUPROALAGLUSRVALTHRARGCYSVALGLYGLYMETSERTHRHISTRPTHRCYSALATHRPROGLUPHEPHELYSGLUASNGLYGLUARGPROLYSILEPHEPROGLYASPGLUTHRLEUASPASPASPGLUTRPLYSLEULEUTYRGLUALAALAARGASNLEUILEGLYVALSERSERTHRGLUPHEASPGLNSERILEARGHISASNTHRVALLEUHISTHRLEUGLNLYSALAPHEPROASNARGGLYILELYSPROLEUPROLEUALACYSHISARGLEUALALYSGLYSERPROTYRVALARGTRPHISALAALAASPASNVALTYRTYRASPLEUPHEASPGLNSERLEUPHEGLYLYSVALASNSERGLUGLYILEALAARGGLYLYSPHEPHELEULEUTHRASNTHRARGCYSTHRLYSLEUHISTHRSERASNPROASNALATHRLYSASPVALASNVALGLYVALALAGLUVALMETASPLEULEUALAASPARGPHETHRGLYSERASPGLNHISLYSGLNVALSERPHEALAILEASNALALYSVALTYRVALVALALAALAGLYALAVALALATHRPROGLNILELEUALAASNSERASPPHEGLYGLYLEUGLUGLYGLNGLNGLYLYSALALEULEUPROALALEUGLYVALGLYILETHRGLUGLNPROLEUALAPHECYSGLNILEILELEUASNGLNHISILEVALASPASPLEULYSASNLEUASNGLYARGPROGLNTRPTRPLYSASPALAVALGLUALAHISARGARGALAHISPROLYSASPPROLEUTRPILEPROPHEGLNASPPROGLUPROGLNVALASNILEPROVALTHRLYSASPPHEPROTRPHISALAGLNILEHISARGASPALAPHESERTYRGLYGLUALAGLYPROARGALAASPSERARGVALVALVALASPLEUARGPHEPHEALAARGGLNALAARGGLUPROLYSASNLYSLEUTHRPHEASPLYSLYSILETHRASPVALTYRGLYMETPROGLNPROTHRPHELYSTYRLEUPROTHRTHRGLNTYRALAASPGLUALAGLYLYSMETMETLYSASPMETTHRGLUVALALASERALALEUGLYGLYTYRLEUPROGLYSERGLUPROGLNPHEMETALAPROGLYLEUALALEUHISLEUGLYGLYTHRVALARGLEUGLYHISGLYSERGLUILEASNGLUSERVALALAASNPHEASNSERGLNVALTRPASNPHELYSASNLEUTYRVALALAGLYASNGLYTHRILEPROTHRALAPHEALAALAASNPROTHRLEUTHRSERILEALALEUALALEUARGALATHRHISHISILEVALLYSVALLEULYSALAASPPROASNASNLEULYSPROALAVALPROGLUGLULYSLEUGLUALATHRPROLYSGLUTYRLEULYSTRPLEUTHRASPPROTHRASNPROASPPHEPROASPHISHISASPLEUGLNLYSGLUHISLYSGLUVALILEVALLYSALAGLUPROSERHISTHRGLYGLYSERGLYSERGLYHISHISHISHISHISHIS

SEQ ID NO: 12 - protein, 1872 aa, Phanerochaete chrysosporium
METPHELEUASPTHRTHRPROPHEARGALAASPGLUPROTYRASPVALPHEILEALAGLYSERGLYPROILEGLYALATHRPHEALALYSLEUCYSVALASPALAASNLEUARGVALCYSMETVALGLUILEGLYALAALAASPSERPHETHRSERLYSPROMETLYSGLYASPPROASNALAPROARGSERVALGLNPHEGLYPROGLYGLNVALPROILEPROGLYTYRHISLYSLYSASNGLUILEGLUTYRGLNLYSASPILEASPARGPHEVALASNVALILELYSGLYALALEUSERTHRCYSSERILEPROTHRSERASNASNHISILEALATHRLEUASPPROSERVALVALSERASNSERLEUASPLYSPROPHEILESERLEUGLYLYSASNPROALAGLNASNPROPHEVALASNLEUGLYALAGLUALAVALTHRARGGLYVALGLYGLYMETSERTHRHISTRPTHRCYSALATHRPROGLUPHEPHEALAPROALAASPPHEASNALAPROHISARGGLUARGPROLYSLEUSERTHRASPALAALAGLUASPALAARGILETRPLYSASPLEUTYRALAGLNALALYSGLUILEILEGLYTHRSERTHRTHRGLUPHEASPHISSERILEARGHISASNLEUVALLEUARGLYSTYRASNASPILEPHEGLNLYSGLUASNVALILEARGGLUPHESERPROLEUPROLEUALACYSHISARGLEUTHRASPPROASPTYRVALGLUTRPHISALATHRASPARGILELEUGLUGLULEUPHETHRASPPROVALLYSARGGLYARGPHETHRLEULEUTHRASNHISARGCYSTHRLYSLEUVALPHELYSHISTYRARGPROGLYGLUGLUASNGLUVALASPTYRALALEUVALGLUASPLEULEUPROHISMETGLNASNPROGLYASNPROALASERVALLYSLYSILETYRALAARGSERTYRVALVALALACYSGLYALAVALALATHRALAGLNVALLEUALAASNSERHISILEPROPROASPASPVALVALILEPROPHEPROGLYGLYGLULYSGLYSERGLYGLYGLYGLUARGASPALATHRILEPROTHRPROLEUMETPROMETLEUGLYLYSTYRILETHRGLUGLNPROMETTHRPHECYSGLNVALVALLEUASPSERSERLEUMETGLUVALVALARGASNPROPROTRPPROGLYLEUASPTRPTRPLYSGLULYSVALALAARGHISVALGLUALAPHEPROASNASPPROILEPROILEPROPHEARGASPPROGLUPROGLNVALTHRILELYSPHETHRGLUGLUHISPROTRPHISVALGLNILEHISARGASPALAPHESERTYRGLYALAVALALAGLUASNMETASPTHRARGVALILEVALASPTYRARGPHEPHEGLYTYRTHRGLUPROGLNGLUALAASNGLULEUVALPHEGLNGLNHISTYRARGASPALATYRASPMETPROGLNPROTHRPHELYSPHETHRMETSERGLNASPASPARGALAARGALAARGARGMETMETASPASPMETCYSASNILEALALEULYSILEGLYGLYTYRLEUPROGLYSERGLUPROGLNPHEMETTHRPROGLYLEUALALEUHISLEUALAGLYTHRTHRARGCYSGLYLEUASPTHRGLNLYSTHRVALGLYASNTHRHISCYSLYSVALHISASNPHEASNASNLEUTYRVALGLYGLYASNGLYVALILEGLUTHRGLYPHEALAALAASNPROTHRLEUTHRSERILECYSTYRALAILEARGALASERASNASPILEILEALALYSPHEGLYARGHISARGGLYGLYGLYSER

SEQ ID NO: 13 - protein, 1839 aa, Aspergillus nidulans
METGLNTYRSERARGMETTHRALATHRARGGLUASNPROLYSTYRLYSASNLEUARGVALGLUGLUCYSASPVALLEUILEILEGLYSERGLYPROVALGLYALATHRTYRALAARGGLUILELEUASPPROGLYSERGLYALASERPROGLYARGLYSALAPROLYSVALILEMETVALGLUTHRGLYALAGLNGLUSERLYSVALPROGLYGLUHISLYSLYSASNALAVALVALTYRGLNLYSHISILEASPSERPHEVALASNVALILEGLNGLYSERLEUPHEALATHRSERVALPROTHRARGVALASPPROASNLEULYSLEUPROPROVALSERTRPSERPROARGGLULYSGLNASNPHEASNGLYGLNASNLYSGLUGLNASNILETYRHISASNLEUASPALAASNGLYVALSERARGASNVALGLYGLYMETSERTHRHISTRPTHRCYSALATHRPROARGGLNHISGLULEUGLUARGSERLYSILEPHEASPASPALATHRTRPASPARGLEUTYRLYSARGALAGLUGLULEUILEGLYTHRARGTHRASPVALLEUASPGLNSERILEARGGLNARGLEUVALLEUASPILELEUARGLYSLYSPHELYSASNARGASPALALYSALALEUPROLEUALAALAGLULYSVALGLUGLYLYSASNLEUILELYSTRPSERSERSERSERTHRVALLEUGLYASNLEULEUGLUASPGLULYSPHETHRLEULEUASPGLNHISHISCYSGLULYSLEUGLUPHEASNASPGLUTHRASNLYSVALSERPHEALAILEILELYSASNLEUALALYSPROGLNTHRSERLYSGLUASPGLUASPARGLEUARGILELYSALALYSTYRVALILEVALCYSGLYGLYPROILELEUTHRPROGLNLEULEUPHELYSSERGLYPHEARGTYRASPGLUGLUASPALAGLUASPSERGLUGLYASNLYSSERSERLEUTYRILEPROALALEUGLYARGASNLEUTHRGLUGLNTHRMETCYSPHECYSGLNILEVALLEULYSASPLYSTRPVALGLUGLULEUGLNLYSASNASNTRPGLYPROGLUCYSGLUGLUHISARGARGLYSTYRASPGLUGLUASPASPPROLEUARGILEPROPHEASPASPLEUASPPROGLNVALTHRLEUPROPHETHRGLUASNTHRPROTRPHISTHRGLNILEHISARGASPALAPHESERTYRGLYALAVALPROPROALAILEASPLYSARGTHRILEVALASPLEUARGTYRPHEGLYARGALAGLUTHRGLNTRPARGASNARGVALTHRPHESERLYSLYSLEUTHRASPALATYRGLYMETPROGLNPROTHRPHEASPPHELYSLEUSERTHRLYSASPARGLEUGLUSERHISARGMETMETGLNASPMETGLULYSVALALAGLYGLULEUGLYGLYTYRLEUPROGLYSERGLUPROGLNPHELEUALAPROGLYLEUALALEUHISVALCYSGLYTHRTHRALAALALEUARGLYSGLYCYSARGSERGLUASPGLUMETLYSARGILESERVALCYSASPGLUASNSERLYSVALTRPGLYVALGLUASNLEUHISLEUGLYGLYLEUASNVALILEPROGLYPROARGSERASNALASERASNPROTHRLEUTHRALAMETCYSPHEALAILELYSGLYALAGLUGLUILEARGARGLYSLEUGLYLYSLYSGLYSERHISSERGLYASNARGASPASPGLYASPVALASPTHRASPTHRASPASPASPALAGLYGLYSERGLYSERGLYHISHISHISHISHISHIS

SEQ ID NO: 14 - protein, 1904 aa, Trametes multicolor
METSERTHRSERSERSERASPPROPHEPHEASNPHEALALYSSERSERPHEARGSERALAALAALAGLNLYSALASERALASERSERLEUPROPROLEUPROGLYPROASPLYSLYSVALPROGLYMETASPILELYSTYRASPVALVALILEVALGLYSERGLYPROILEGLYCYSTHRTYRALAARGGLULEUVALGLYALAGLYTYRLYSVALALAMETPHEASPILEGLYGLUILEASPSERGLYLEULYSILEGLYALAHISLYSLYSASNTHRVALGLUTYRGLNLYSASNILEASPLYSPHEVALASNVALILEGLNGLYGLNLEUMETSERVALSERVALPROVALASNTHRLEUVALVALASPTHRLEUSERPROTHRSERTRPGLNALASERTHRPHEPHEVALARGASNGLYSERASNPROGLUGLNASPPROLEUARGASNLEUSERGLYGLNALAVALTHRARGVALVALGLYGLYMETSERTHRHISTRPTHRCYSALATHRPROARGPHEASPARGGLUGLNARGPROLEULEUVALLYSASPASPALAASPALAASPASPALAGLUTRPASPARGLEUTYRTHRLYSALAGLUSERTYRPHEGLNTHRGLYTHRASPGLNPHELYSGLUSERILEARGHISASNLEUVALLEUASNLYSLEUTHRGLUGLUTYRLYSGLYGLNARGASPPHEGLNGLNILEPROLEUALAALATHRARGARGSERPROTHRPHEVALGLUTRPSERSERALAASNTHRVALPHEASPLEUGLNASNARGPROASNTHRASPALAPROGLUGLUARGPHEASNLEUPHEPROALAVALALACYSGLUARGVALVALARGASNALALEUASNSERGLUILEGLUSERLEUHISILEHISASPLEUILESERGLYASPARGPHEGLUILELYSALAASPVALTYRVALLEUTHRALAGLYALAVALHISASNTHRGLNLEULEUVALASNSERGLYPHEGLYGLNLEUGLYARGPROASNPROALAASNPROPROGLULEULEUPROSERLEUGLYSERTYRILETHRGLUGLNSERLEUVALPHECYSGLNTHRVALMETSERTHRGLULEUILEASPSERVALLYSSERASPMETTHRILEARGGLYTHRPROGLYGLULEUTHRTYRSERVALTHRTYRTHRPROGLYALASERTHRASNLYSHISPROASPTRPTRPASNGLULYSVALLYSASNHISMETMETGLNHISGLNGLUASPPROLEUPROILEPROPHEGLUASPPROGLUPROGLNVALTHRTHRLEUPHEGLNPROSERHISPROTRPHISTHRGLNILEHISARGASPALAPHESERTYRGLYALAVALGLNGLNSERILEASPSERARGLEUILEVALASPTRPARGPHEPHEGLYARGTHRGLUPROLYSGLUGLUASNLYSLEUTRPPHESERASPLYSILETHRASPALATYRASNMETPROGLNPROTHRPHEASPPHEARGPHEPROALAGLYARGTHRSERLYSGLUALAGLUASPMETMETTHRASPMETCYSVALMETSERALALYSLEGLYGLYPHELEUPROGLYSERLEUPROGLNPHEMETGLUPROGLYLEUVALLEUHISLEUGLYGLYTHRHISARGMETGLYPHEASPGLULYSGLUASPASNCYSCYSVALASNTHRASPSERARGVALPHEGLYPHELYSASNLEUPHELEUGLYGLYCYSGLYASNILEPROTHRALATYRGLYALAASNPROTHRLEUTHRALAMETSERLEUALAILELYSSERCYSGLUTYRILELYSGLNASNPHETHRPROSERPROPHETHRSERGLUALAGLNGLYGLYSERGLYSERGLYHISHISHISHISHISHIS

SEQ ID NO: 15 - protein, 657 aa, synthetic construct
MGHHHHHHSSGHIEGRHMASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDFEDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 16 - protein, 657 aa, synthetic construct
MGHHHHHHSSGHIEGRHMASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 17 - protein, 647 aa, synthetic construct
GAOMRQWSASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ